(19) United States
(12) Patent Application Publication
(10) Pub. No.: US 2003/0166190 A1
(43) Pub. Date: Sep. 4, 2003

(54) Nucleic acids related to plant retroelements

(76) Inventors: David A. Wright, Bonne, IA (US); Daniel F. Voytas, Ames, IA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/315,515

(22) Filed: Dec. 10, 2002

Related U.S. Application Data
(60) Provisional application No. 60/339,060, filed on Dec. 10, 2001.

(51) Int. Cl. C12N 9/00; C12N 9/24; C07H 21/04; C07K 14/415

(52) U.S. Cl. 435/183; 530/350; 435/200; 536/23.2; 530/370

(57) ABSTRACT

The invention provides a family of plant retroelements as well as nucleic acids, vectors, and polypeptides relating to those retroelements.

### CROSS-REFERENCE TO RELATED APPLICATIONS

This application claims priority to U.S. Provisional Application Serial No. 60/339,060, filed Dec. 10, 2001, which is hereby incorporated by reference in its entirety for all purposes.

### STATEMENT AS TO FEDERALLY SPONSORED RESEARCH

Funding for the work described herein was provided in part by the federal government, National Institutes of Health grant number GM61420. The federal government may have certain rights in the invention.

### FIELD OF THE INVENTION

The invention relates to nucleic acids having homology to retroelements. More particularly, the invention relates to nucleic acids having homology to a family of retrovirus elements from Arabidopsis thaliana.

### BACKGROUND OF THE INVENTION

Retroelements have been identified in every eukaryote in which they have been sought. A retroelement essentially is a DNA that can be transcribed, reverse transcribed, and integrated into a new genomic location. Replication by reverse transcription is responsible for much of the repetitive DNA found in the eukaryotic genome. Retroelements can be divided into two major classes: the Long Terminal Repeat (LTR) elements and the non-LTR elements.

LTR elements typically encode a polyprotein that is proteolytically cleaved into functional subunits. The primary proteins are Group Specific Antigens (Gag) and Polymerase (Pol). Gag proteins form the structural components of the particulate replication intermediate. Gag proteins aggregate together during initial assembly and are cleaved into smaller subunits to form a mature particle. Pol is cleaved into protease, reverse transcriptase, and integrase. These Pol proteins work within the particle. Protease extracts itself from the polyprotein and processes the other proteins. Reverse transcriptase is responsible for cDNA synthesis, and integrase inserts the cDNA into the host genome. The LTR retroelements are divided into the retroviruses and retrotransposons. The primary difference between the groups is that retroviruses can leave their host cell via their envelope (Env) protein and retrotransposons are trapped within their host cell primarily because they lack a functional Env protein.

Flanking the gag/pol coding region are several cis-acting DNA sequences that assist in replication. These are the LTRs, Primer Binding Site (PBS), PolyPurine Tract (PPT) and the mRNA packaging signal. Although the LTRs are identical in sequence, they serve different functions. The 5′ LTR acts as the promoter, whereas the 3′ LTR provides the polyadenylation signal and the polyadenylation site. The PBS and PPT act as primer sites for the initiation of DNA synthesis, and the packaging signal ensures that the viral RNA is taken into the particle.

Retroelement proliferation can be directly or indirectly associated with disease. Many retroviruses cause disease directly by interfering with normal cellular function upon infection. Retrotransposons are usually benign but can cause mutations by gene disruption, duplication, deletion, or by altering gene activity.

In a few instances, retroelements have been harnessed by their host cells to perform a specific function. An example is found in Drosophila melanogaster, where the elements HeT-A and Tart have taken over the role of telomerase in telomere maintenance (Levis et al. (1993) Cell 75: 1083-1093). Additionally, it is thought that the env gene of an endogenous retrovirus is used during human placenta development to produce syncytia, which are multinucleated cells formed by the fusion of fetal cells (Mi et al. (2000) Nature 403: 785-789; Blond et al. (2000) J. Virol. 74: 3321-3329). Over evolutionary time, the benefits of such retroelement activity have outweighed any detrimental consequences and such retroelements have not been eliminated from the genome.

Historically, retroviruses were thought to be limited in their distribution to vertebrates because they had only been observed as disease causing agents of vertebrates. However, several non-vertebrate retroelements have been described that have retrovirus-like features. For example, some non-vertebrate retroelements appear to encode an Env-like protein. Additionally, a series of experiments has shown that a D. melanogaster element called gypsy is an insect retrovirus. Crude cell, and pupal extracts from cells that express gypsy, and purified gypsy virus-like particles (VLPs) have been demonstrated to cause infection of D. melanogaster strains that do not have active gypsy elements (Pelisson et al (1994) EMBO J. 13: 4401-4411; Song et al. (1994) Genes Dev. 8: 2046-2057). It also was found that antibodies against gypsy env blocked viral infection (Song et al. (1994) supra).

The Athila and SIRE retroelements were the first plant retroelements to be described that have the potential to be retroviruses (Laten et al. (1998) Genetica 107: 87-93; Wright and Voytas (1998) Genetics 149: 703-715). Since the characterization of Athila and SIRE, other plant retrovirus-like elements have been identified. The element Cyclops was identified in Pisum sativum (Chavanne et al. (1998) Plant Mol. Biol. 37: 363-75), and Calypso was found in Glycine max along with retrovirus-like elements from Oryza sativa, Sorghum bicolor, Avena sativa, Secale cereale, Horcleum vulgare, Triticum aestivum, Gossypium hirsutum, Platanus occidentalis, Lycopersicon esculentum, Solanum tuberosum, and Nicotiana tabacum (Wright and Voytas (2002) Genome Res. 12: 122-131).

### SUMMARY OF THE INVENTION

The invention provides novel nucleic acids having homology to plant retroelements as well as segments of those retroelements, including LTRs, promoters, LTR end sequences, Gag/Pol nucleic acids and polypeptides, integrase nucleic acids and polypeptides, protease nucleic acids and polypeptides, reverse transcriptase nucleic acids and polypeptides, and envelope nucleic acids and polypeptides.

In one aspect, the invention features an isolated Athila retroelement containing a nucleic acid having a nucleotide sequence that is at least 90% identical to the nucleotide sequence set forth in SEQ ID NO:122, or the complement thereof.

In another aspect, the invention features an isolated nucleic acid encoding a polypeptide, wherein the polypeptide has an amino acid sequence that is at least 85% identical to the amino acid sequence set forth in SEQ ID NO:128. The nucleic acid sequence can encode a Gag/Pol polypeptide.

The invention also features an isolated nucleic acid containing a nucleotide sequence that is at least 90% identical to nucleotides 1 to 1747 or 12220 to 13966 of the sequence set forth in SEQ ID NO:122, or the complement thereof. The nucleotide sequence can function as a Long Terminal Repeat.

In another aspect, the invention features an isolated nucleic acid containing a nucleotide sequence that is at least 90% identical to nucleotides 1 to 385 of the sequence set forth in SEQ ID NO:122, or the complement thereof. The nucleotide sequence can function as a promoter.

In another aspect, the invention features an isolated nucleic acid containing a nucleotide sequence that is at least 90% identical to nucleotides 1 to 40 or 1708 to 1747 of the sequence set forth in SEQ ID NO:122, or the complement thereof. The nucleotide sequence can function as an LTR-end sequence.

In another aspect, the invention features an isolated nucleic acid encoding a polypeptide, wherein the polypeptide has an amino acid sequence that is at least 85% identical to the amino acid sequence set forth in SEQ ID NO:140. The polypeptide can be a functional Gag polypeptide.

In yet another aspect, the invention features an isolated nucleic acid containing a nucleotide sequence that is at least 90% identical to nucleotides 1893 to 3575 of the sequence set forth in SEQ ID NO:122, or the complement thereof. The nucleic acid can encode a functional Gag polypeptide.

In another aspect, the invention features an isolated nucleic acid encoding a polypeptide, wherein the polypeptide has an amino acid sequence that is at least 85% identical to the amino acid sequence set forth in SEQ ID NO:141. The polypeptide can function as a protease polypeptide.

The invention also features an isolated nucleic acid containing a nucleotide sequence that is at least 90% identical to nucleotides 3576 to 4556 of the sequence set forth in SEQ ID NO:122, or the complement thereof. The nucleic acid can encode a functional protease polypeptide.

In another aspect, the invention features an isolated nucleic acid encoding a polypeptide, wherein the polypeptide has an amino acid sequence that is at least 85% identical to the amino acid sequence set forth in SEQ ID NO:139. The polypeptide can function as a reverse transcriptase polypeptide.

In another aspect, the invention features an isolated nucleic acid containing a nucleotide sequence that is at least 90% identical to nucleotides 4602 to 6314 of the sequence set forth in SEQ ID NO:122, or the complement thereof. The nucleic acid can encode a functional reverse transcriptase polypeptide.

In another aspect, the invention features an isolated nucleic acid encoding a polypeptide, wherein the polypeptide has an amino acid sequence that is at least 85% identical to the amino acid sequence set forth in SEQ ID NO:142. The polypeptide can function as an integrase polypeptide.

In yet another aspect, the invention features an isolated nucleic acid containing a nucleotide sequence that is at least 90% identical to nucleotides 6315 to 7625 of the sequence set forth in SEQ ID NO:122, or the complement thereof The nucleic acid can encode a functional integrase polypeptide.

In still another aspect, the invention features an isolated nucleic acid encoding a polypeptide, wherein the polypeptide has an amino acid sequence that is at least 85% identical to the amino acid sequence set forth in SEQ ID NO:129, SEQ ID NO:130, or SEQ ID NO:131. The polypeptide can function as an envelope polypeptide.

The invention also features an isolated nucleic acid containing a nucleotide sequence that is at least 90% identical to nucleotides 8745 to 10600, nucleotides 8745 to 10673, or nucleotides 8745 to 10728 of the sequence set forth in SEQ ID NO:122, or the complement thereof. The nucleic acid can encode a functional envelope polypeptide.

Any of the isolated nucleic acids disclosed above can be at least 91 percent, at least 92 percent, at least 93 percent, at least 94 percent, at least 95 percent, at least 96 percent, at least 97 percent, at least 98 percent, at least 99 percent, or more than 99 percent identical to the nucleotide sequence set forth in SEQ ID NO:122, a portion thereof, or the complement thereof.

In another aspect, the invention features a purified polypeptide containing an amino acid sequence that is at least 85 percent identical to the amino acid sequence set forth in SEQ ID NO:140. The polypeptide can function as a Gag polypeptide.

In another aspect, the invention features a purified polypeptide containing an amino acid sequence that is at least 85 percent identical to the amino acid sequence set forth in SEQ ID NO:141. The polypeptide can function as a protease polypeptide.

The invention also features a purified polypeptide containing an amino acid sequence that is at least 85 percent identical to the amino acid sequence set forth in SEQ ID NO:139. The polypeptide can function as a reverse transcriptase polypeptide.

In another aspect, the invention features a purified polypeptide containing an amino acid sequence that is at least 85 percent identical to the amino acid sequence set forth in SEQ ID NO:142. The polypeptide can function as an integrase polypeptide.

In still another aspect, the invention features a purified polypeptide containing an amino acid sequence that is at least 85 percent identical to the amino acid sequence set forth in SEQ ID NO:129, SEQ ID NO:130, or SEQ ID NO:131. The polypeptide can function as an envelope polypeptide.

Any of the purified polypeptides described above can be at least 86 percent, at least 87 percent, at least 88 percent, at least 89 percent, at least 90 percent, at least 91 percent, at least 92 percent, at least 93 percent, at least 93 percent, at least 94 percent, at least 95 percent, at least 96 percent, at least 97 percent, at least 98 percent, at least 99 percent, or more than 99 percent identical to any of the amino acid sequences set forth in a SEQ ID NO provided herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a neighbor-joining tree of reverse transcriptases from A. thaliana Ty3-gypsy retroelements. Each major group (i.e., classic, Tat, and Athila) is labeled. Numbers along the branches indicate bootstrap support for 100 replicates. Arrows indicate the most recent common ancestor for each of the three lineages.

FIG. 2 is an illustration of the structural organization of A. thaliana Athila4 elements. Boxes with filled triangles represent LTRs. Open boxes represent coding sequences, and are offset to indicate changes in reading frame. Vertical thin lines represent stop codons. Horizontal thin lines represent non-coding sequences. The shaded region identifies the coding region for reverse transcriptase. Shaded boxes indicate env. The accession number, BAC designator and position within the BAC for each Athila4 element are as follows: Athila4-1, AC007209, F1404, 33315 to 47208; Athila4-2, AB026642, MED5, 3448 to 17452; Athila4-3 and Athila4-4, AC007534, F7F22, 88613 to 114709; Athila4-5, AL353871, F7K15, 86117 to 99436; Athila4-6, AF296831, F1809, 38836 to 52851.

FIG. 3A is a comparison of PBS sequences from Athila1-1, Athila2-1, Athila4-1, Athila5-1, Athila6-1, Calypso1-1, Calypso5-1, Cyclops-2, Rice and BAGY-2 (SEQ ID NOS:1 to 10, respectively). FIG. 3A also illustrates that these sequences are complimentary to the 3′ end of the Asp tRNA (SEQ ID NO:11). Complementary sequences are shaded, including those that form G:U base pairs. FIG. 3B provides a comparison of PPT sequences from Athila1-3, Athila2-1, Athila3-1, Athila4-1, Athila5-1, Athila6-1, Calypso2-1, Calypso2-1#2, Calypso4-1, Cyclops-2, and Rice that are found after the env-like ORF (PPT 1; SEQ ID NOS:12 to 22, respectively) and near the 3′ LTR (PPT; SEQ ID NOS:23 to 33, respectively). A conserved core sequence motif is shaded.

FIG. 4A is an illustration of the structural organization of Athila4 and Calypso consensus elements with individual related elements from pea (Cyclops-2), barley (BAGY-2), rice (positions 36238 to 53391; an inserted element was removed for the diagram), and soybean (Diaspora). Below the Cyclops-2 structure is a graph depicting amino acid identity along the length of gag-pol amino acid sequence. Shading indicates location of protease (PR), gray indicates reverse transcriptase (RT), and dark gray indicates integrase (IN). All other aspects of the figure are as described for FIG. 2. FIG. 4B is an illustration of amino acid sequence signatures of gag-pol proteins from the Athila1-1, Athila4-1, Athila5-1, Athila6-1, Calypso1-1, Calypso2-1, Calypso3-1, Calypso4-1, Calypso5-1, Cyclops-2, Rice, BAGY-2, and Diaspora retroelements (SEQ ID NOS:34 to 46, respectively). The sequence domains are identified. Motifs are shown that define conserved domains of reverse transcriptase (Xiong and Eickbush (1990) EMBO J. 9: 3353-3362). For integrase, the zinc binding domain is shown, as are signatures for the DD35E domain (Fayet et al. (1990) Mol. Microbiol. 4: 1771-1777) and the GPY/F motif (Malik and Eickbush (1999) J. Virol. 73: 5186-5190).

FIG. 5A is an illustration of the general organization of env-like ORFs from the A. thaliana Athila group elements, the soybean Calypso elements, Cyclops-2 of pea, gypsy of D. melanogaster and HIV1. Open boxes indicate ORFs. Arrows indicate signal sequences. Black boxes indicate transmembrane domains. Vertical lines within boxes denote stop codons. The first methionine within each ORF is indicated by a short line. FIG. 5B is an amino acid sequence comparison of N-terminal signal sequences from the Athila2-1, Athila3-1, Athila4-1, Athila6-3, Athila1-1, Athila5, Calypso1-1, Calypso2-1, Calypso3-1, Calypso4-1, Calypso5-1, and Cyclops-2 retroelements (SEQ ID NOS:47 to 58, respectively); transmembrane domain 1 (TM1) sequences from the Athila2-1, Athila3-1, Athila4-1, Athila6-3, Calypso1-1, Calypso2-1, Calypso3-1, and Calypso5-1 retroelements (SEQ ID NOS:59 to 66); TM2 sequences from the Athila2-1, Athila3-1, Athila4-1, Athila6-3, Athila1-1, Athila5, Calypso2-1, Calypso3-1, and Calypso4-1 retroelements (SEQ ID NOS:67 to 75, respectively); and TM3 sequences from the Athila2-1, Athila3-1, Athila4-1, Athila6-3, Athila1-1, Athila5, Calypso2-1, and Calypso3-1 retroelements (SEQ ID NOS:76 to 83, respectively). FIG. 5C is TMpred output graphs for the Athila4 consensus env-like ORF with and without a frameshift at the C-terminus. Values above 500 (on the X-axis) are significant and indicate likely transmembrane domains. The Y-axis indicates amino acid sequence position. FIG. 5D is a nucleotide sequence comparison of the putative splice acceptor site of the Athila1-1, Athila2-1, Athila3-1, Athila4-1, Athila4-2, Athila4-3, Athila4-4, Athila4-5, Athila4-6, Athila4-10, Calypso1-1, Calypso2-1, Calypso3-1, Calypso4-1, Calypso5-1, Calypso7-1, and Calypso8-1 elements (SEQ ID NOS:84 to 100, respectively). Confidence levels indicate the output for NetGene 2 (Brunak et al. (1991) J. Mol. Biol. 220: 49-65; Hebsgaard et al. (1996) Nucleic Acids Res. 24: 3439-3452). The first methionine in each env-like ORF is in bold.

FIG. 6 is a sequence comparison of transcription termination sites of A. thaliana Athila clones that match the Athila6 and Athila4 group elements. Sequences are given for Athila6-1, pDW777, pDW778, pDW779, pDW776, Athila4-1, pDW775, pDW774, pDW827, pDW826, pDW824, pDW823, pDW821, pDW832, pDW820, F03G22, pDW825, F2112, pDW780, and T17A2 (SEQ ID NOS:101 to 120, respectively). At the top of the diagram is a generic Athila LTR with the region denoted wherein the transcripts terminate. Numbers next to the arrows indicate the base position for transcription termination sites within the LTR.

FIG. 7 is an alignment of the Athila4 element with the consensus Athila4-1 element (SEQ ID NOS:121 and 122, respectively). Changes that were made in Athila4-1 to construct a consensus Athila4 virus are indicated by asterisks. Numbers under “Athila” in the sequence alignment refer to changes that were made in the original mutant Athila4-1 sequence. “DVO” followed by a number designates a specific oligonucleotide primer that was used to introduce changes to the mutant Athila4-1 sequence by PCR site directed mutagenesis.

FIG. 8 is a nucleotide sequence alignment of six Athila4 group elements (Athila4-4, Athila4-5, Athila4-3, Athila4-1, Athila4-2, and Athila4-6; SEQ ID NOS:123, 124, 125, 121, 126, and 127, respectively) with an Athila4 consensus sequence (SEQ ID NO:122).

FIG. 9 provides consensus nucleotide (SEQ ID NO:122) and amino sequences (SEQ ID NOS:128-131) for the entire Athila4 Arabidopsis thaliana retroelement. Protein coding regions are translated, with stop codons represented by Z. Three Env-like amino acid sequences result from readthrough of a stop codon (SEQ ID NO:130) and a frame shift (SEQ ID NO:131), in addition to the expected amino acid sequence (SEQ ID NO:129).

FIG. 10 is an amino acid alignment of gag/pol sequences from six Athila4 group elements (Athila4-5, Athila4-4, Athila4-6, Athila4-1, Athila4-2, and Athila4-3; SEQ ID NOS:132 to 137, respectively) with an Athila4 consensus amino acid sequence of the gag/pol sequence (SEQ ID NO:128).

FIG. 11 provides a consensus nucleotide (SEQ ID NO:138) and amino acid sequences (SEQ ID NO:139) for an active an Athila4 reverse transcriptase (pJR3). Stop codon signals are represented by Z. Positions of the four nucleotide changes made to produce the functional reverse transcriptase are marked in bold.

FIG. 12 is a graph plotting radioactive nucleotide incorporation in Counts Per Minute (CPM) by reverse transcription of an RNA template. AMV RT is a positive control, Wheat Germ Extract is the background level of RT in the translation mixture, Boiled Wheat Germ Extract is the background after boiling to destroy RT activity, Wheat Germ Extract plus Athila4 mRNA is the activity of the Athila4 RT plus the background from the translation mixture, and the Boiled Wheat Germ Extract plus Athila4 mRNA shows the RT activity level after boiling.

FIG. 13 is a graph depicting the radioactive nucleotide incorporation in CPM by reverse transcription of an RNA template. The standard error is shown at the top of each bar. AMV RT is a positive control, Wheat Germ Extract is the background level of RT in the translation mixture, Boiled Wheat Germ Extract is the background after boiling to destroy RT activity, Wheat Germ Extract plus Athila4 mRNA is the activity of the Athila4 RT plus the background from the translation mixture, and the Boiled Wheat Germ Extract plus Athila4 mRNA shows the RT activity level after boiling.

FIG. 14 is a photograph of a western blot demonstrating protease activity.

### DETAILED DESCRIPTION OF THE INVENTION

Definitions

The term “amino acid sequence” refers to the positional arrangement and identity of amino acids in a peptide, polypeptide, or protein molecule. Use of the term “amino acid sequence” is not meant to limit the amino acid sequence to the complete, native amino acid sequence of a peptide, polypeptide or protein.

“Chimeric” is used to indicate that a DNA sequence, such as a vector or a gene, is comprised of more than one DNA sequences of distinct origin with are fused together by recombinant DNA techniques resulting in a DNA sequence, which does not occur naturally.

The term “coding region” refers to the nucleotide sequence that codes for a protein of interest. The coding region of a protein is bounded on the 5′ side by the nucleotide triplet “ATG” which encodes the initiator methionine and on the 3′ side by one of the three triplets that specify stop codons (i.e., TAA, TAG, and TGA).

“Constitutive expression” refers to expression using a constitutive promoter.

“Constitutive promoter” refers to a promoter that is able to express the gene that it controls in all, or nearly all, phases of the life cycle of the cell.

“Complementary” or “complementarity” is used to define the degree of base-pairing or hybridization between nucleic acids. For example, as is known to one of skill in the art, adenine (A) can form hydrogen bonds or base pair with thymine (T) and guanine (G) can form hydrogen bonds or base pair with cytosine (C). Hence, A is complementary to T and G is complementary to C. Complementarity may be complete when all bases in a double-stranded nucleic acid are base paired. Alternatively, complementarity may be “partial,” in which only some of the bases in a nucleic acid are matched according to the base pairing rules. The degree of complementarity between nucleic acid strands has an effect on the efficiency and strength of hybridization between nucleic acid strands.

The “derivative” of a reference nucleic acid, protein, polypeptide, or peptide has a related but different sequence or chemical structure than the respective reference nucleic acid, protein, polypeptide, or peptide. A derivative nucleic acid, protein, polypeptide, or peptide generally is made purposefully to enhance or incorporate some chemical, physical, or functional property that is absent or only weakly present in the reference nucleic acid, protein, polypeptide, or peptide. A derivative nucleic acid generally can differ in nucleotide sequence from a reference nucleic acid, whereas a derivative protein, polypeptide, or peptide can differ in amino acid sequence from the reference protein, polypeptide or peptide, respectively. Such sequence differences can include one or more substitutions, insertions, additions, deletions, fusions, and/or truncations, which can be present in any combination. Differences can be minor (e.g. a difference of one nucleotide or amino acid) or more substantial. However, the sequence of the derivative is not so different from the reference that one of skill in the art would not recognize that the derivative and reference are related in structure and/or function. Generally, differences are limited so that the reference and the derivative are closely similar overall and, in many regions, identical. A “variant” differs from a “derivative” nucleic acid, protein, polypeptide or peptide in that the variant can have silent structural differences that do not significantly change the chemical, physical or functional properties of the reference nucleic acid, protein, polypeptide or peptide. In contrast, the differences between the reference and derivative nucleic acid, protein, polypeptide or peptide are intentional changes made to improve one or more chemical, physical, or functional properties of the reference nucleic acid, protein, polypeptide, or peptide.

“Expression” refers to the transcription and/or translation of a structural gene.

“Expression cassette” means a nucleic acid sequence capable of directing expression of a particular nucleic acid. Expression cassettes generally contain a promoter operably linked to the nucleic acid to be expressed (e.g., a coding region), which also is operably linked to termination signals. Expression cassettes also can contain other nucleic acid segments as desired for proper transcription and translation of the nucleic acid, for example, under particular conditions or as needed for transcription and/or translation of the particular nucleic acid in a particular host cell.

“Genome” refers to the complete genetic material that is naturally present in an organism and is transmitted from one generation to the next.

“Heterologous nucleic acid” refers to a nucleic acid that originates from a source that is foreign to the particular virus or host or, if from the same source, a heterologous nucleic acid is modified from its original form. The term also includes non-naturally occurring multiple copies of a naturally occurring nucleic acid. Thus, the term refers to a nucleic acid segment that is foreign or heterologous to the virus or cell, or normally found within the virus or cell but in a position within the genome where it is not ordinarily found.

“Homology,” as used herein, refers to the identity of nucleotide and/or amino acid sequences. As is understood in the art, nucleotide mismatches can occur at the third or wobble base in the codon without causing amino acid substitutions in the final polypeptide sequence. Also, minor nucleotide modifications (e.g., substitutions, insertions or deletions) in certain regions of the gene sequence can be tolerated and considered insignificant whenever such modifications result in changes in amino acid sequence that do not alter the functionality of the final product. It has been shown that chemically synthesized copies of whole or partial gene sequences can replace the corresponding regions in the natural gene without loss of gene function. Homologs of specific DNA sequences may be identified by those skilled in the art using the test of cross-hybridization of nucleic acids under conditions of stringency as is well understood in the art (as described in Hames and Higgins (eds.) Nucleic Acid Hybridization, IRL Press, Oxford, UK (1985). Extent of homology often is measured in terms of percentage of identity between the sequences compared. Thus, in this disclosure it will be understood that minor sequence variation can exist within homologous sequences.

“Hybridization” refers to the process of annealing complementary nucleic acid strands by forming hydrogen bonds between nucleotide bases on complementary nucleic acid strands. Hybridization, and the strength of the association between the nucleic acids, is impacted by such factors as the degree of complementary between the hybridizing nucleic acids, the stringency of the conditions involved, the Tₘ of the formed hybrid, and the G:C ratio within the nucleic acids.

“Inducible promoter” refers to a regulated promoter that can be turned on in one or more cell types by an external stimulus, such as a chemical, light, hormone, stress, temperature or a pathogen.

An “initiation site” is region surrounding the position of the first nucleotide that is part of the transcribed sequence, which is defined as position +1. All nucleotide positions of the gene are numbered by reference to the first nucleotide of the transcribed sequence, which resides within the initiation site. Downstream sequences (i.e., sequences in the 3′ direction) are denominated positive, while upstream sequences (i.e., sequences in the 5′ direction) are denominated negative.

“Introns” or “intervening sequences” refer to those regions of DNA sequence that are transcribed along with the coding sequences (exons) but are then removed in the formation of the mature mRNA. Introns may occur anywhere within a transcribed sequence—between coding sequences of the same or different genes, within the coding sequence of a gene, interrupting and splitting its amino acid sequences, and within the promoter region (5′ to the translation start site). Introns in a primary transcript are excised and the coding sequences are simultaneously and precisely ligated to form mature mRNA. The junctions of introns and exons form the splice sites. The base sequence of an intron typically begins with GU and ends with AG. The same splicing signal is found in many higher eukaryotes.

“Leader sequence” refers to a DNA sequence that typically contains about 100 nucleotides and is located between the transcription start site and the translation start site. A leader sequence also contains a region that specifies the ribosome binding site.

The terms “open reading frame” and “ORF” refer to the amino acid sequence encoded between translation initiation and termination codons of a coding sequence. The terms “initiation codon” and “termination codon” refer to units of three adjacent nucleotides (‘codons’) in a coding sequence that specify initiation and chain termination, respectively, of protein synthesis (mRNA translation).

“Operably linked” means two or more nucleic acids are joined to form one nucleic acid molecule, so that the function of one is affected by the other. In general, “operably linked” also means that two or more nucleic acids are suitably positioned and oriented so that they can function together. Nucleic acids often are operably linked to permit transcription of a coding region to be initiated from the promoter. For example, a regulatory sequence is the to be “operably linked to” or “associated with” a DNA sequence that codes for an RNA or a polypeptide if the two sequences are situated such that the regulatory sequence affects expression of the coding region (i.e., the coding sequence or functional RNA is under the transcriptional control of the promoter). Coding regions can be operably-linked to regulatory sequences in sense or antisense orientation.

“Plant tissue” includes differentiated and undifferentiated tissues of plants, including, but not limited to roots, shoots, leaves, pollen, seeds, tumor tissue and various forms of cells in culture, such as single cells, protoplasts, embryos and callus tissue. The plant tissue may be in a plant or in an organ, tissue or cell culture.

“Polyadenylation signal” refers to any nucleic acid sequence capable of effecting mRNA processing, usually characterized by the addition of polyadenylic acid tracts to the 3′-ends of the mRNA precursors. The polyadenylation signal DNA segment may itself be a composite of segments derived from several sources, naturally occurring or synthetic, and may be from a genomic DNA or an RNA-derived cDNA. Polyadenylation signals are commonly recognized by homology to the canonical form 5′-AATAA-3′, although variation of distance, partial “readthrough,” and multiple tandem canonical sequences are not uncommon. A polyadenylation signal may in fact cause transcriptional termination and not polyadenylation (Montell et al. (1983) Nature 305:600-605).

“Promoter” refers to the nucleotide sequences at the 5′ end of a structural gene that direct the initiation of transcription. Promoter sequences are necessary, but not always sufficient, to drive the expression of a downstream gene. In general, eukaryotic promoters include a characteristic DNA sequence homologous to the consensus 5′-TATAAT-3′ (TATA) box about 10-30 bp 5′ to the transcription start (cap) site, which, by convention, is numbered +1. Bases 3′ to the cap site are given positive numbers, whereas bases 5′ to the cap site receive negative numbers, reflecting their distance from the cap site. Another promoter component, the CAAT box, often is found about 30 to 70 bp 5′ to the TATA box and has homology to the canonical form 5′-CCAAT-3′ (Breathnach and Chambon (1981) Ann. Rev. Biochem. 50: 349-383). In plants the CAAT box is sometimes replaced by a sequence known as the AGGA box, a region having adenine residues symmetrically flanking the triplet G(orT)NG (Messing et al. (1983), in Genetic Engineering of Plants, Kosuge, Meredith and Hollaender (eds.), Plenum Press, pp. 211-227). Other sequences conferring regulatory influences on transcription can be found within the promoter region and extending as far as 1000 bp or more 5′ from the cap site.

The terms “protein,” “peptide,” and “polypeptide” are used interchangeably herein.

“Regulatory sequences” and “regulatory elements” refer to segments of nucleic acids that control some aspect of the expression of another nucleic acid segment. Such sequences or elements can be located upstream (5′ non-coding sequences), within, or downstream (3′ non-coding sequences) of a coding sequence. Regulatory sequences and regulatory elements influence the transcription, RNA processing or stability, or translation of the associated coding sequence. Regulatory sequences include enhancers, introns, promoters, polyadenylation signal sequences, splicing signals, termination signals, and translation leader sequences. They also include natural and synthetic sequences.

“Selectable marker” refers to a gene that encodes an observable or selectable trait that is expressed and can be detected in an organism having that gene. Selectable markers often are linked to a nucleic acid of interest that may not encode an observable trait, in order to trace or select the presence of the nucleic acid of interest. Any selectable marker known to one of skill in the art can be used with the nucleic acids of the invention. Some selectable markers allow the host to survive under circumstances in which, without the marker, the host would otherwise die. Examples of selectable markers are provided herein.

As used herein the term “stringency” is used to define the conditions of temperature, ionic strength, and the presence of other compounds such as organic solvents, under which nucleic acid hybridizations are conducted. With “high stringency” conditions, nucleic acid base pairing will occur only between nucleic acids that have a high frequency of complementary base sequences. “Weak” or “low” stringency conditions typically are used for nucleic acids in which the frequency of complementary sequences is lower, so that nucleic acids with differing sequences can be detected and/or isolated.

The terms “substantially similar” and “substantially homologous” refer to nucleotide and amino acid sequences that represent functional equivalents of the nucleic acids of the invention. For example, altered nucleotide sequences which simply reflect the degeneracy of the genetic code but nonetheless encode amino acid sequences that are identical to the inventive amino acid sequences are substantially similar to the inventive sequences. In addition, nucleic acids that are substantially similar to the nucleic acids of the invention can encode proteins with sufficient overall amino acid identity to function in a manner similar to the reference protein. For example, nucleic acid sequences that are substantially similar to the sequences of the invention can be those wherein the overall amino acid identity is 65% or greater, 70% or greater, 75% or greater, 80% or greater, 90% or greater, or 95% or greater relative to the nucleic acid sequences identified by the SEQ ID NOS provided herein.

A “variant” of a reference nucleic acid, protein, polypeptide or peptide, has a related but different sequence than the reference nucleic acid, protein, polypeptide, or peptide, respectively. The differences between variant and reference nucleic acids, proteins, polypeptides, or peptides are silent or conservative differences. A variant nucleic acid differs in nucleotide sequence from a reference nucleic acid, whereas a variant protein, polypeptide, or peptide differs in amino acid sequence from the reference protein, polypeptide, or peptide, respectively. A variant and reference nucleic acid, protein, polypeptide or peptide may differ in sequence by one or more substitutions, insertions, additions, deletions, fusions, and/or truncations, which may be present in any combination. Differences can be minor (e.g., a difference of one nucleotide or amino acid) or more substantial. However, the structure and function of the variant is not so different from the reference that one of skill in the art would not recognize that the variant and reference are related in structure and/or function. Generally, differences are limited so that the reference and the variant are closely similar overall and, in many regions, identical.

The term “vector” is used to refer to a nucleic acid that can transfer another nucleic acid segment(s) into a cell. A vector includes, inter alia, any plasmid, cosmid, phage, viral or other nucleic acid in double- or single-stranded, linear or circular form which may or may not be self transmissible or mobilizable, and which can transform prokaryotic or eukaryotic host cells either by integration into the cellular genome or by existing extrachromosomally (e.g. autonomously replicating plasmids with an origin of replication). Vectors used in bacterial systems often contain an origin of replication that allows the vector to replicate independently of the bacterial chromosome. The term “expression vector” refers to a vector containing an expression cassette.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the term “variant” or “derivative” refers to a gene or gene product that displays modifications in sequence and or functional properties (i.e., altered characteristics) when compared to the wild-type gene or gene product. Naturally occurring derivatives can be isolated; these are identified by the fact that they have altered characteristics when compared to the wild-type gene or gene product.

Athila Retroelements

A 5′ LTR and a 3′ LTR flank the body of an Athila retroelement. One LTR serves as a promoter for RNA polymerase II and the other provides signals for transcript termination. An LTR typically begins with TG, ends with CA and is bound by a short inverted repeat. An LTR is divided into three discrete sections. The unique 3′ region (U3) is the 5′ end of the LTR but is found at the 3′ end of the mRNA. The U3 region usually contains the enhancers, silencers, promoter and polyadenylation signals. The redundant region (R) follows the U3 region within the LTR. The R region is found at both ends of the mRNA and is delineated by the transcription start site and a polyadenylation site. The unique 5′ region (U5) is the 3′ end of the LTR, is found near the 5′ end of the mRNA (after R) and may contain regulatory sequences as well. The lengths of U3, R, and U5 vary among the different retroelements. Accordingly, LTRs generally function to regulate transcription of mRNA, and LTRs more specifically include promoter, polyadenylation, enhancer, and silencer functions. The ability of a particular nucleotide sequence to function as LTR can be assessed by, for example, determining the ability of the sequence to direct transcription, as described in Example 2, for example.

During the life cycle of a retrovirus, a single, long mRNA is produced by transcription of a retroviral genome. This mRNA functions as a template for translation and later as a template for reverse transcription. The mRNA usually encodes all of the proteins that are required for replication, typically this includes the Gag and Pol proteins.

The PBS is a cis-acting sequence that lies between the 5′ LTR and the coding region. The PBS is complementary to a specific tRNA that is used as a primer to initiate first strand synthesis during reverse transcription. The RT helps produce a complementary DNA copy of the retroviral mRNA by binding to the tRNA primer and the PBS.

The PPT is located between the coding region and the 3′LTR. An Athila4 retroelement typically contains two conserved polypurine tracts, Polypurine Tract 1 (PPT1), and Polypurine Tract 2 (PPT2). The polypurine tract defines an RNase H resistant region that is used to prime second (plus) strand synthesis during replication. PPT 1 resides within the Athila4 retroelement at about 12205 to about position 12218, and PPT2 is at about positions 10738 to 10747.

The packaging signal is an additional mRNA sequence feature used to promote packaging. This packaging signal is a sequence in or near gag that is recognized by the retroelement proteins and promotes packaging of the mRNA into the developing virus or virus-like particle (VLP). A mature retroelement mRNA contains the following regions 5′ to 3′: Cap, R, U5, PBS, packaging signal, gag/pol coding region, polypurine tract, U3, R and a poly A tail.

The minimal complement of proteins for all self-propagating LTR retroelements are the proteins encoded by the gag and pol genes. The Pol polyprotein encodes PR, RT, and IN proteins, whereas the Gag polyprotein forms a virus or virus-like particle. The Gag polyprotein is cleaved into subunits, but not until maturation of the virus-like particle.

Because more Gag than Pol is required for virus-like particles, the ratio of Gag to Pol proteins is regulated to ensure proper virus-like particle formation. The Gag and Pol polyproteins can be encoded either in separate ORFs or in a single ORF. Such ORFs typically are separated by a frameshift or a stop codon. In this way, the Pol protein is only made when the translation machinery switches reading frames, or reads through an intervening stop codon, thus ensuring that more Gag than Pol protein is produced. It is thought that the Pol protein is preferentially degraded or the Gag protein may be translated from a spliced RNA in retrovirus or retrovirus-like retroelements that encode Gag and Pol in a single ORF.

A virus-like particle begins as a complex of immature Gag and Gag/Pol fusion proteins in the cytoplasm. As a particle forms, retroelement mRNA and specific tRNAs are taken inside in preparation for maturation of the virus-like particle. The protease excises itself from the Gag/Pol fusion protein and cleaves the remaining proteins into their functional forms, thereby producing a mature particle. After a particle matures, an unknown factor (possibly involved in cell division) stimulates the reverse transcription process.

LTR sequences at the ends of the mRNA are essential for a series of template transfers during reverse transcription. Reverse transcription begins with a specific primer, usually a tRNA, which binds to the PBS. Association of the tRNA and the RT with the PBS may occur at the same time. RT then catalyzes synthesis of a DNA complementary to the length of the mRNA that is called a Minus Strong Stop DNA (−ssDNA). The Minus Strong Stop DNA includes the R, U5, and the RNA primer sequences. After polymerization, the Minus Strong Stop DNA dissociates from the RNA.

The R region on the Minus Strong Stop DNA is complementary to the R region on the 3′ end of the mRNA. After hybridization between the Minus Strong Stop DNA and the R region of the 3′ end of the mRNA, first strand DNA synthesis is carried out through to the 5′ R region. The mRNA is degraded by the ribonuclease H (RNase H) function of the RT, except for a small piece of the PPT. This small polypurine RNA fragment is used to prime Plus Strong Stop DNA (+ssDNA) synthesis.

The Plus Strong Stop DNA forms a complete LTR, which includes the polypurine tract RNA, U3, R and U5 LTR regions. The Plus Strong Stop DNA is now complementary to the 5′ end of the first strand of DNA. The 3′ end of the Plus Strong Stop DNA then is used to extend the second strand to the end of the DNA template. The reverse transcription process reforms the complete 5′ and 3′ LTRs, creating a blunt-ended cDNA molecule.

The cDNA has a 2 to 3 base extension on each end that was created on the 5′ end by nucleotides in the 5′ LTR during initiation of DNA synthesis by the primer at the PBS, and on the 3′ end by nucleotides in the 3′ LTR during initiation of DNA synthesis by the PPT. The resulting retroelement cDNA is then ready for the integration step that inserts the cDNA into the genome of the host cell.

Integration begins when the integrase protein, and possibly other proteins, form an integration complex that binds the ends of the retroelement cDNA. The 3′ ends of each strand of the cDNA are recessed, usually by about two nucleotides, and a 3′ OH is exposed. The integration complex has access to the host genome only during cell division when the nuclear membrane is dissolved. However, some retroelements are able to transport the integration complex across the nuclear membrane. Once the host DNA is accessible, the integration complex picks a target, which could be bent DNA, as is the preferred case for some retroviruses, or specific targets that seem to be particular to different retrotransposons.

The integration complex binds the target DNA and the 3′ OH groups of the cDNA are used to attack the phosphodiester bonds of the host DNA target site. The attacks occur four to six bases apart, to produce a staggered cut, and the cDNA 3′ ends are joined to the host DNA. The mismatching 5′ ends are recessed and the gaps are filled in by cellular repair mechanisms or possibly by reverse transcriptase. The repair produces a Target Site Duplication (TSD) at both ends of the retroelement, which is a hallmark of integration. At this point the new retroelement DNA is ready to start the life cycle over again.

The major feature that distinguishes the retroviruses from the retrotransposons is a coding sequence for an envelope (Env) protein. The Env protein bestows infectivity to the retrovirus particles (Coffin et al., (1997) Retroviruses. Cold Spring Harbor Laboratory Press (Cold Spring Harbor, N.Y.)). Although env genes are not well conserved at the primary sequence level, they do share a number of common features. For example, most env genes are encoded by spliced subgenomic mRNAs. Also, Env proteins typically have a signal peptide (for targeting to the membranes of the endoplasmic reticulum) along with a central and a C-terminal transmembrane domain. Moreover, Env proteins are processed post-translationally by proteolytic cleavage to generate surface and transmembrane proteins. In addition, the surface and transmembrane subunits of Env proteins often are glycosylated and are joined together via noncovalent or disulfide bonds. Furthermore, mature Env proteins are embedded in the plasma membrane via the C-terminal transmembrane domain.

Unlike retrotransposon Gag proteins, retrovirus Gag proteins are associated with the cell membrane. Type C particles actually form in direct association with the membrane, while type B and D particles begin to organize in the cytoplasm after which the core particle moves to the cell membrane. The immature retrovirus is encased in a membrane bilayer containing Env proteins as it buds off from the cell. Shortly after assembling, the retrovirus assumes the mature form. Env proteins mediate infection by interacting with receptors on the surface of target cells, causing membrane fusion and release of the core particle within the target cell. The retrovirus then goes through the same steps that a retrotransposon goes through, including reverse transcription and integration.

Nucleic Acids

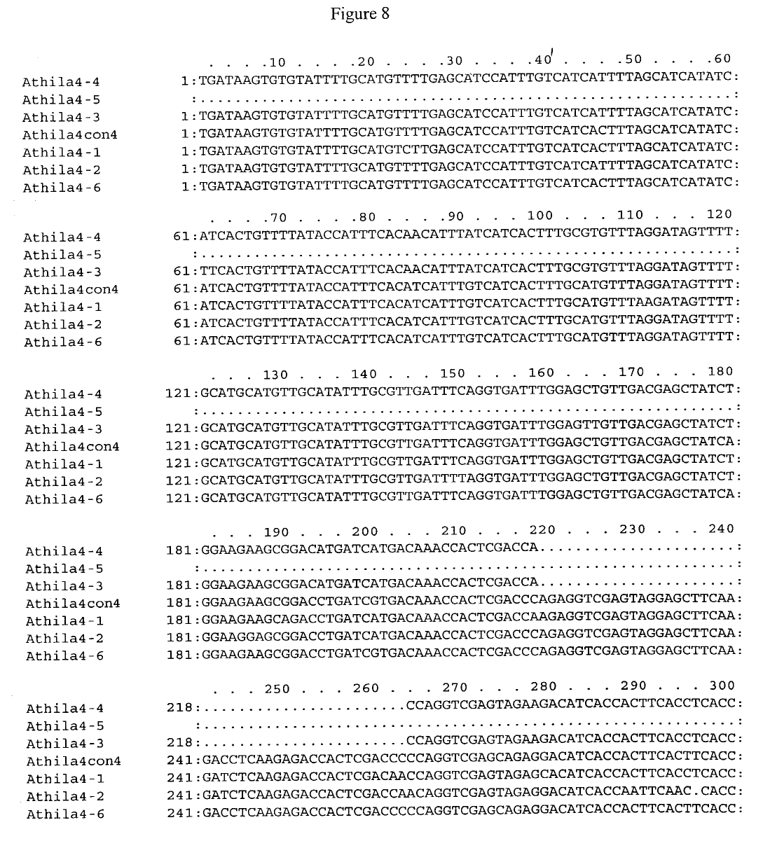

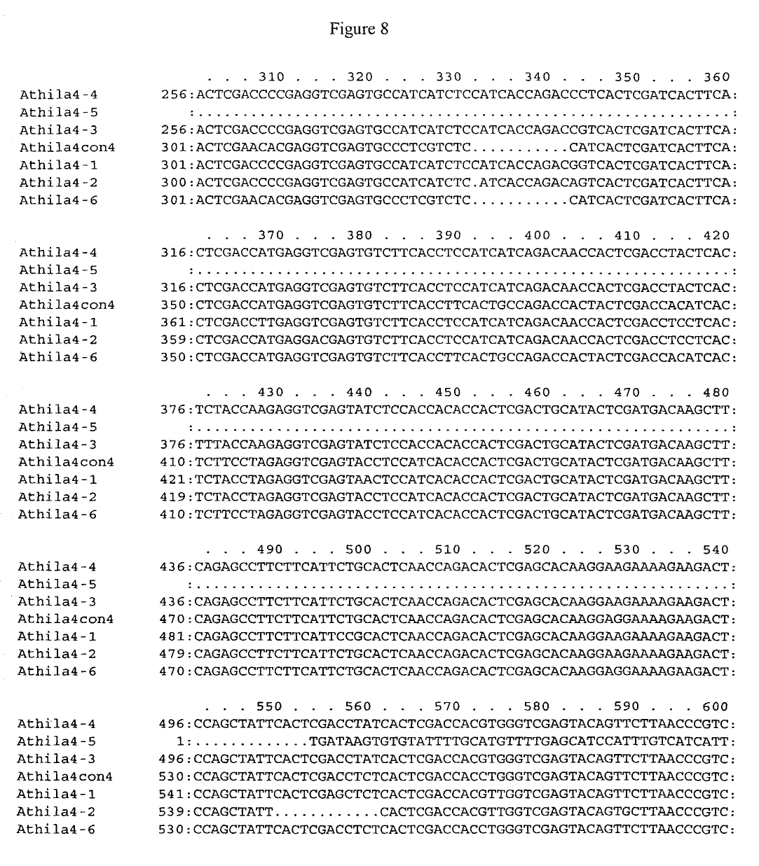

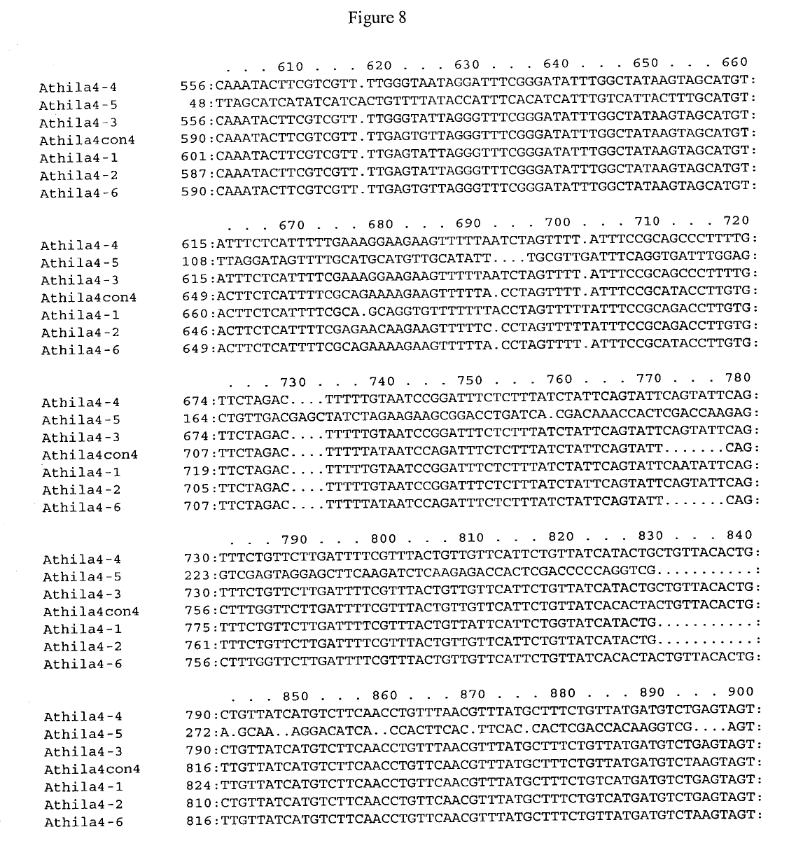

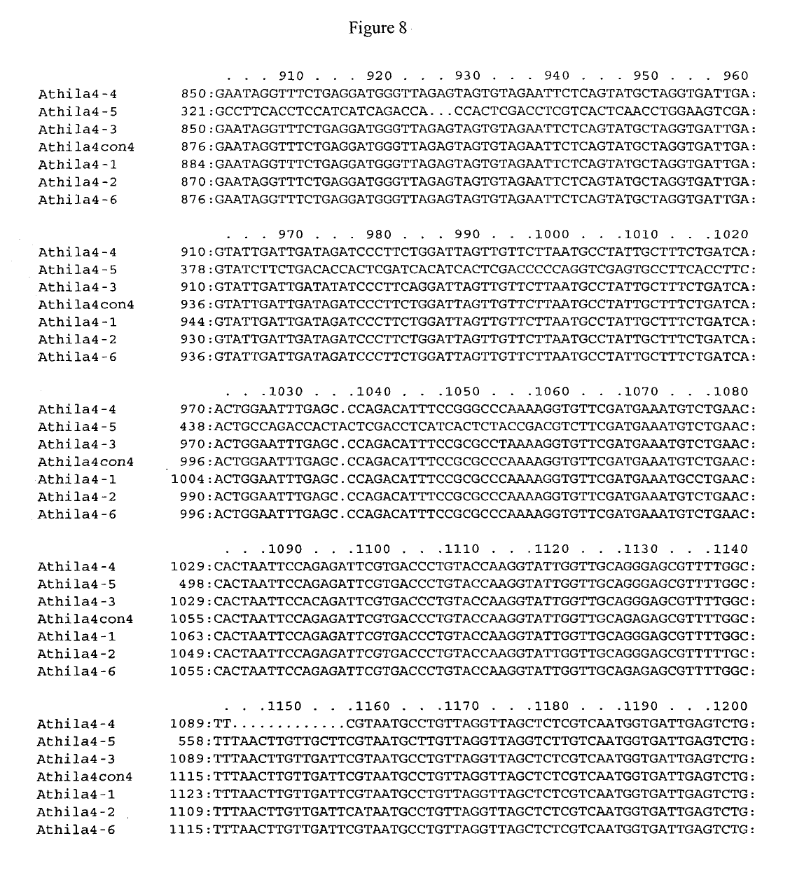

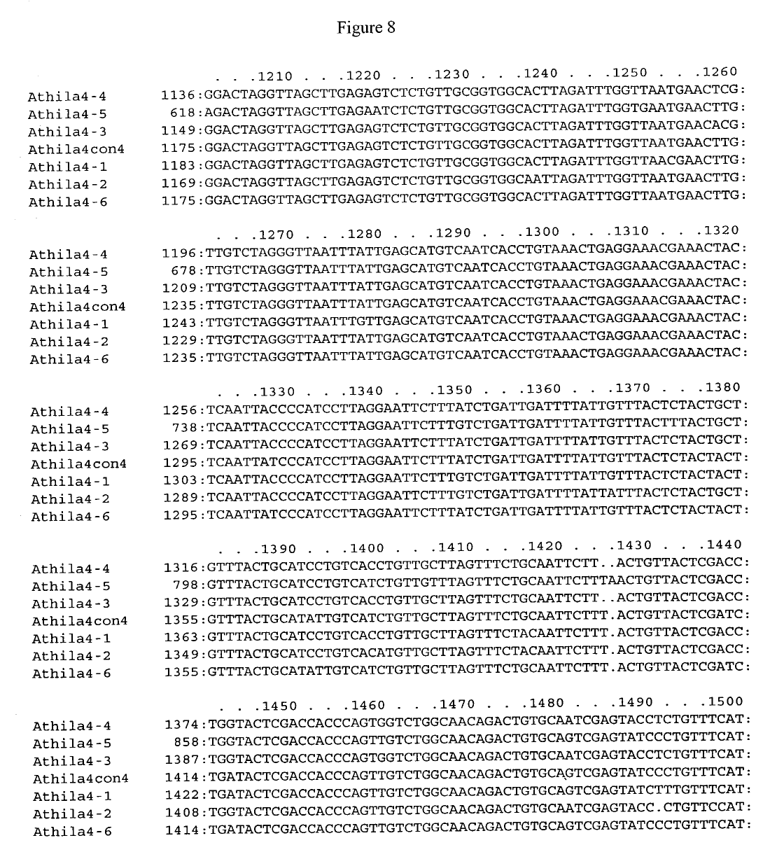

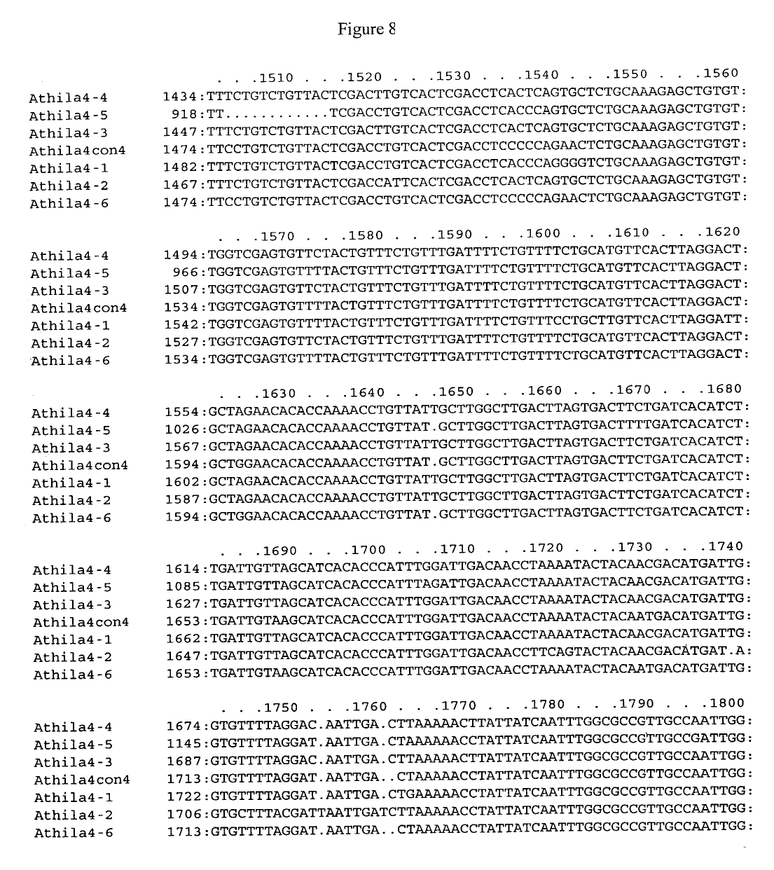

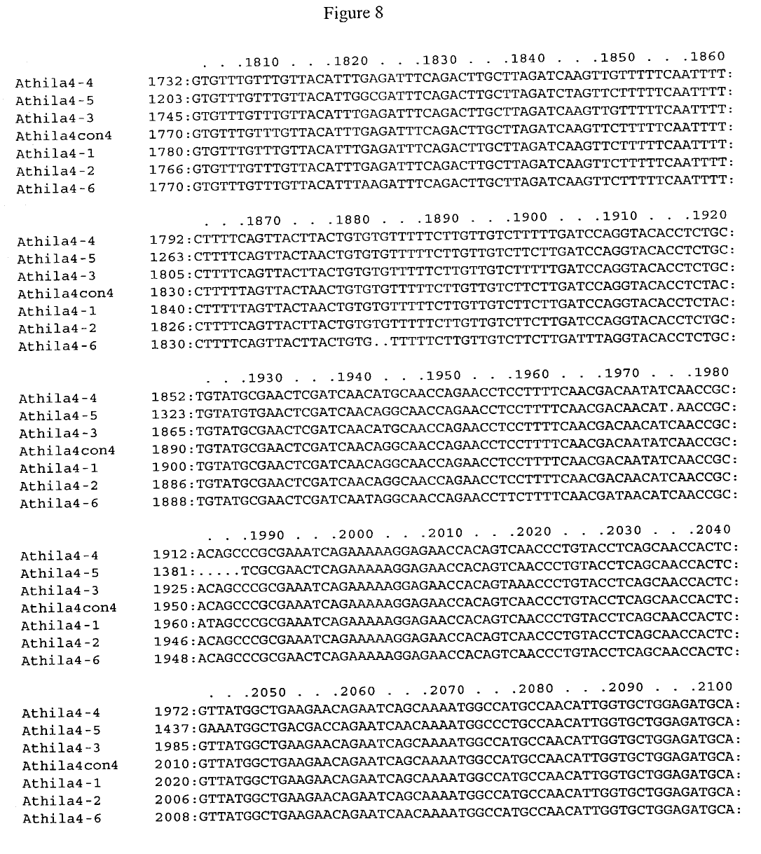

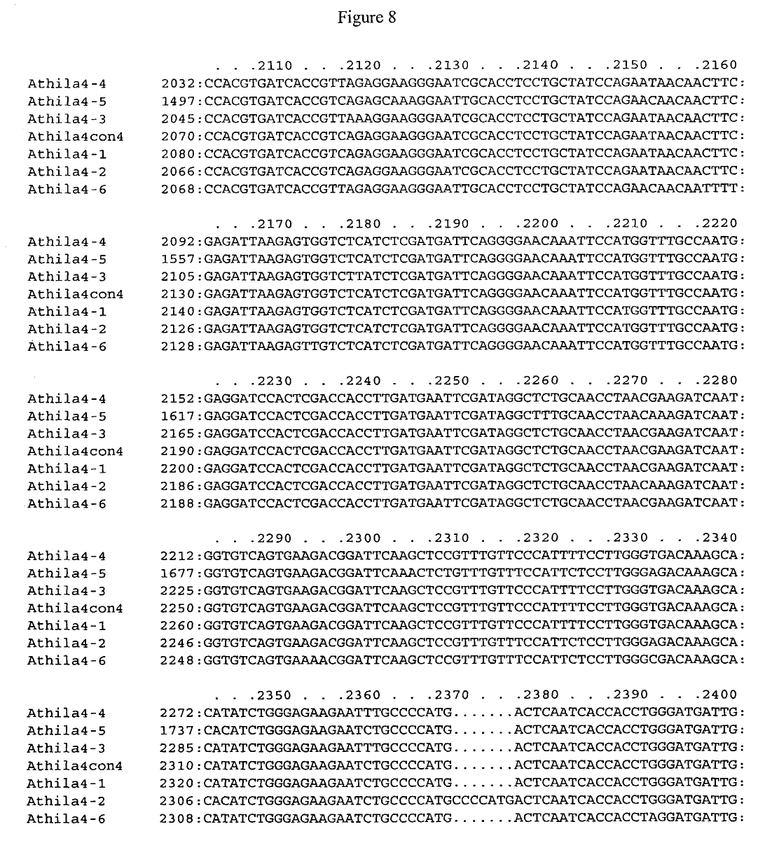

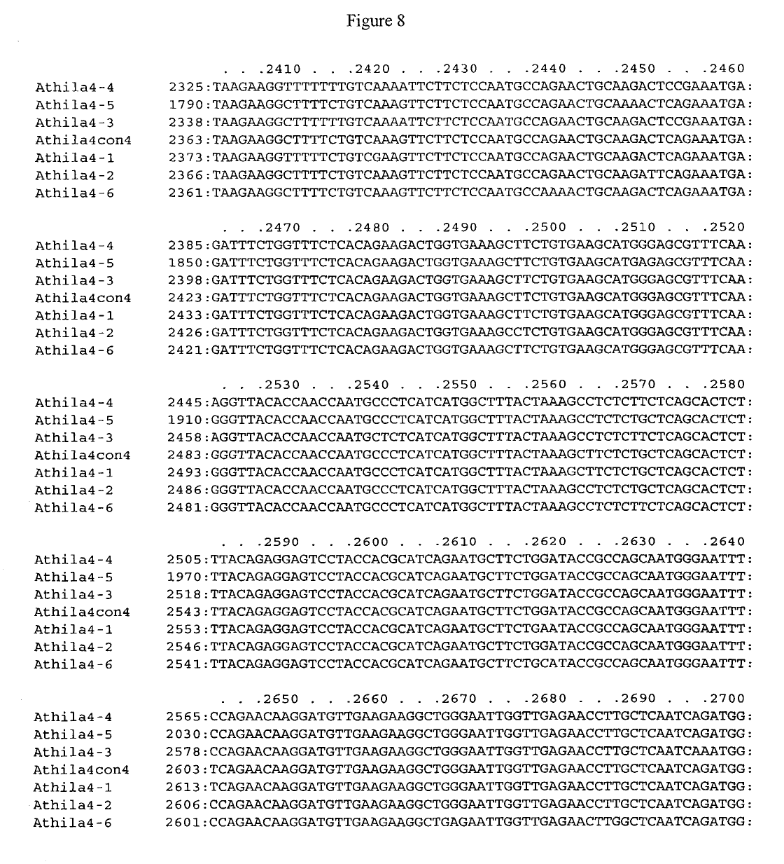

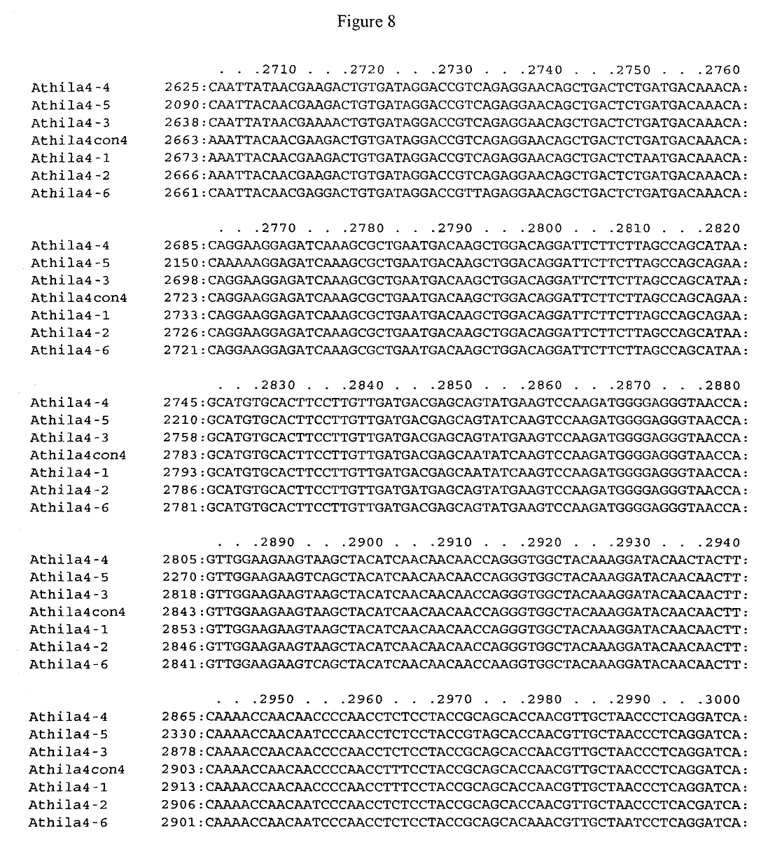

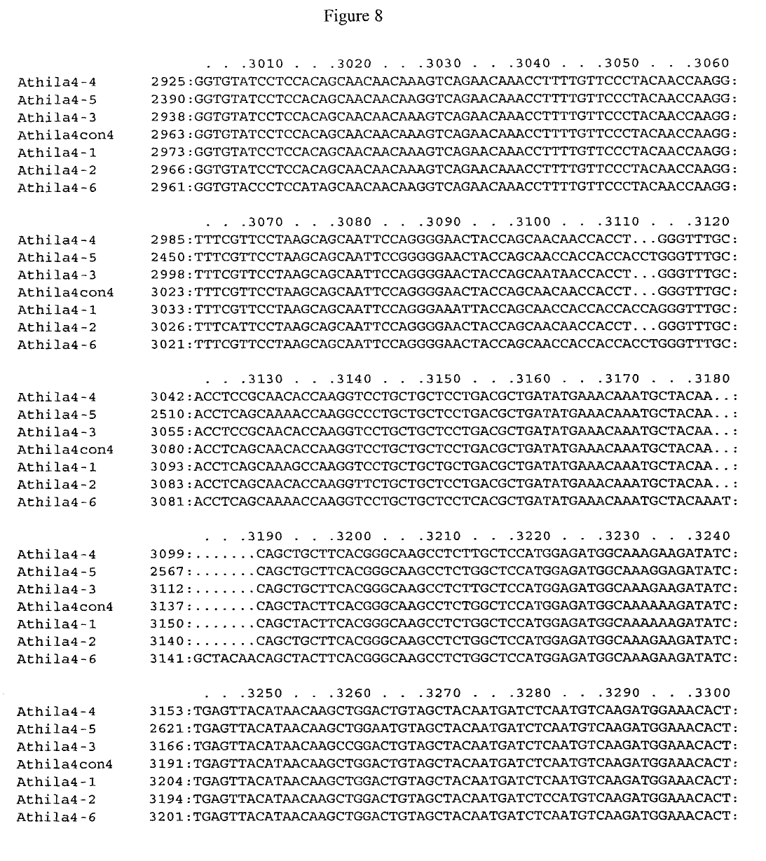

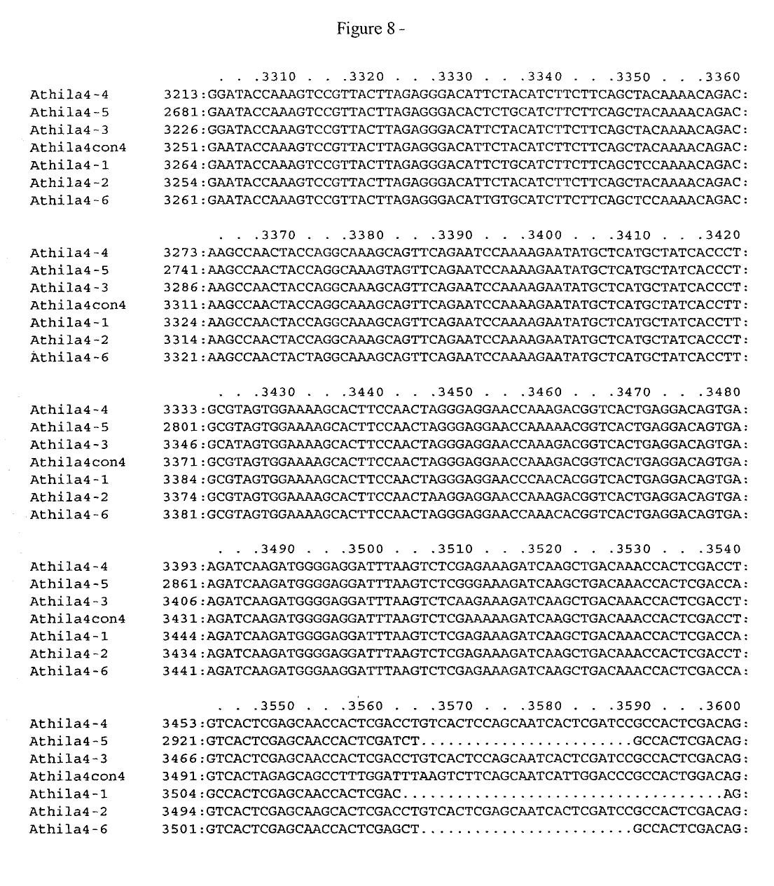

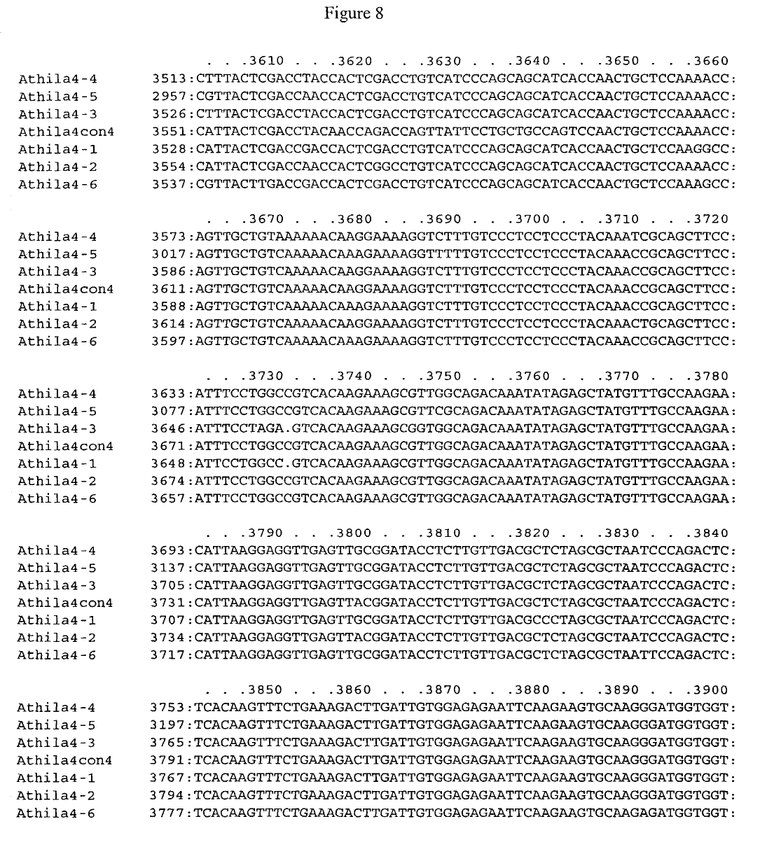

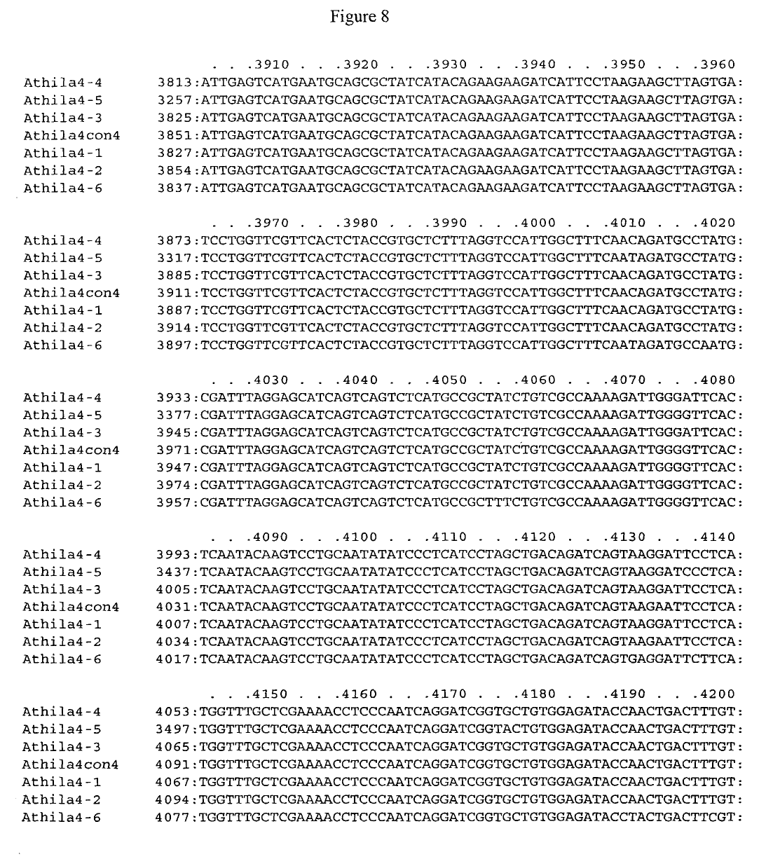

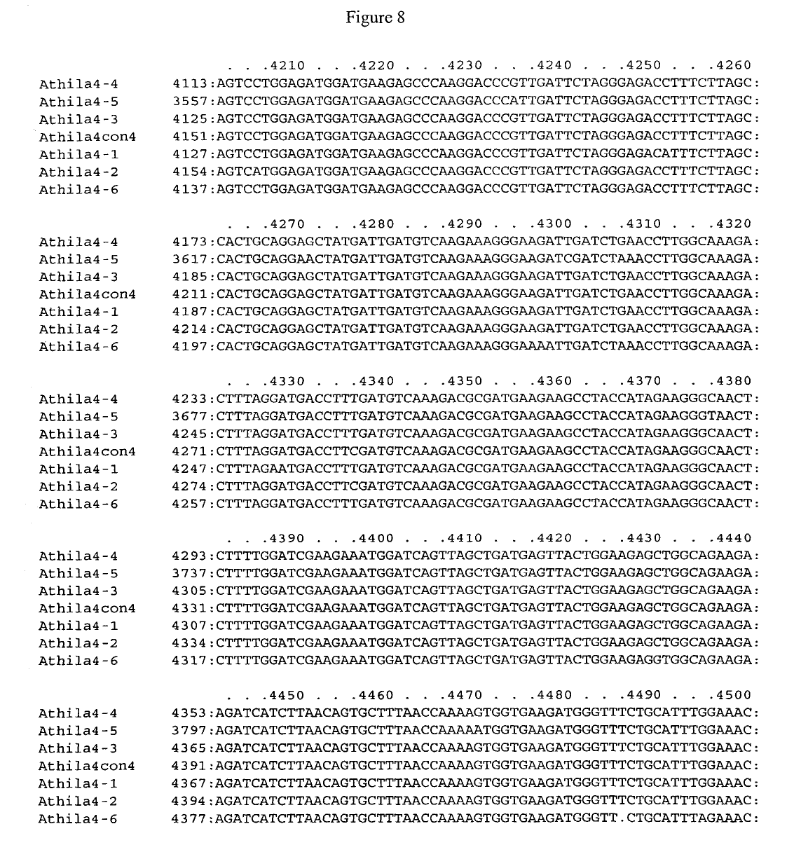

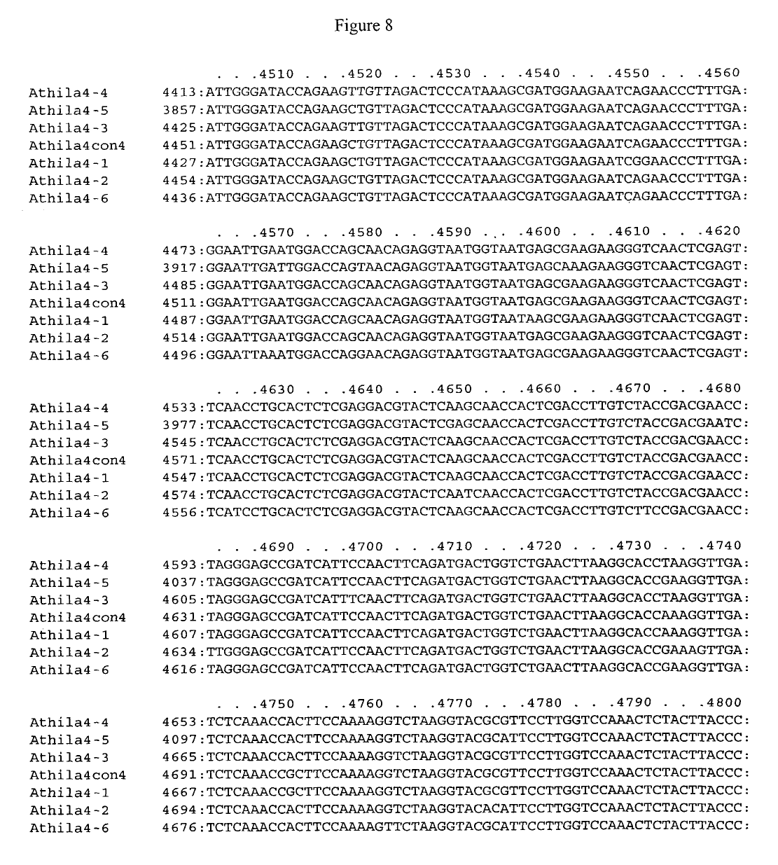

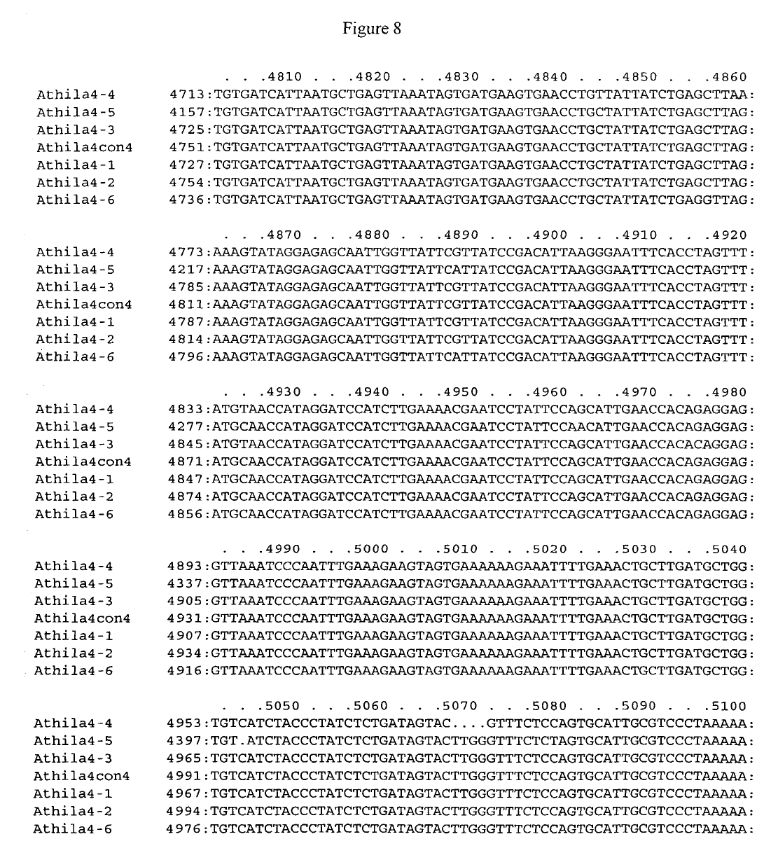

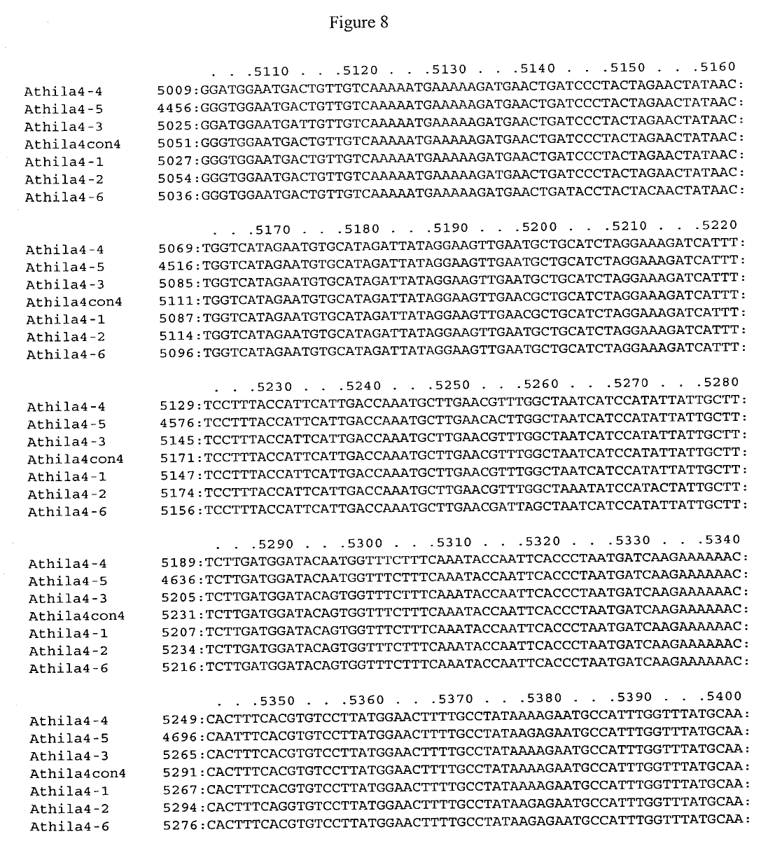

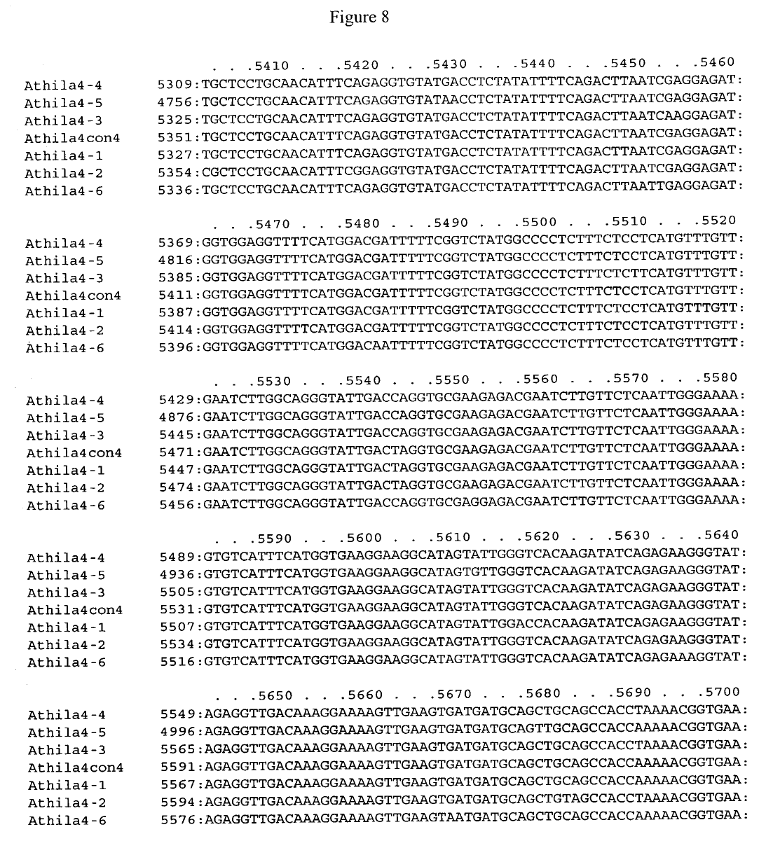

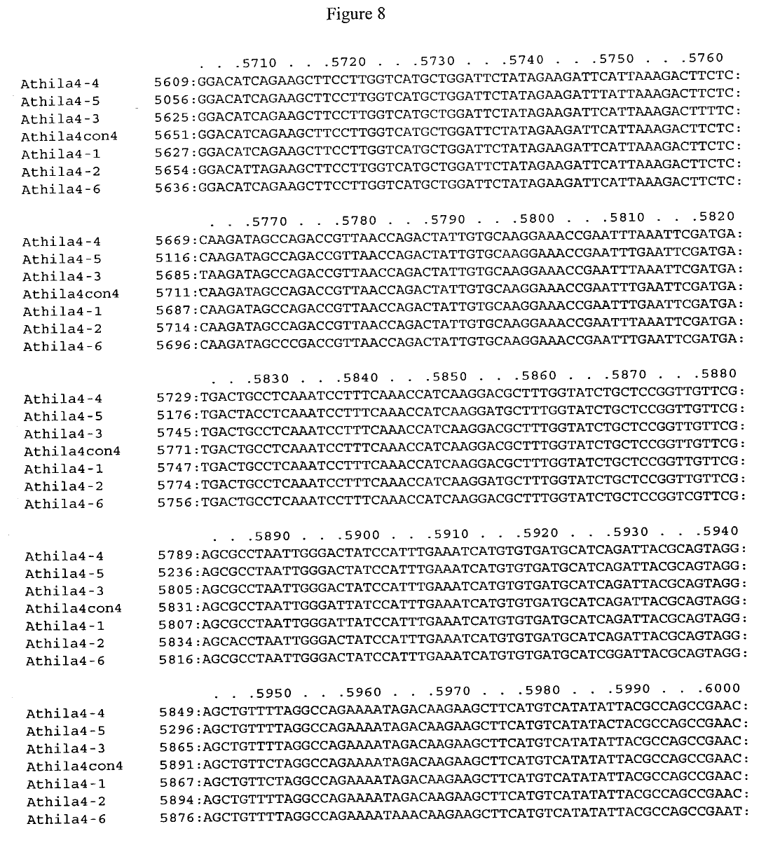

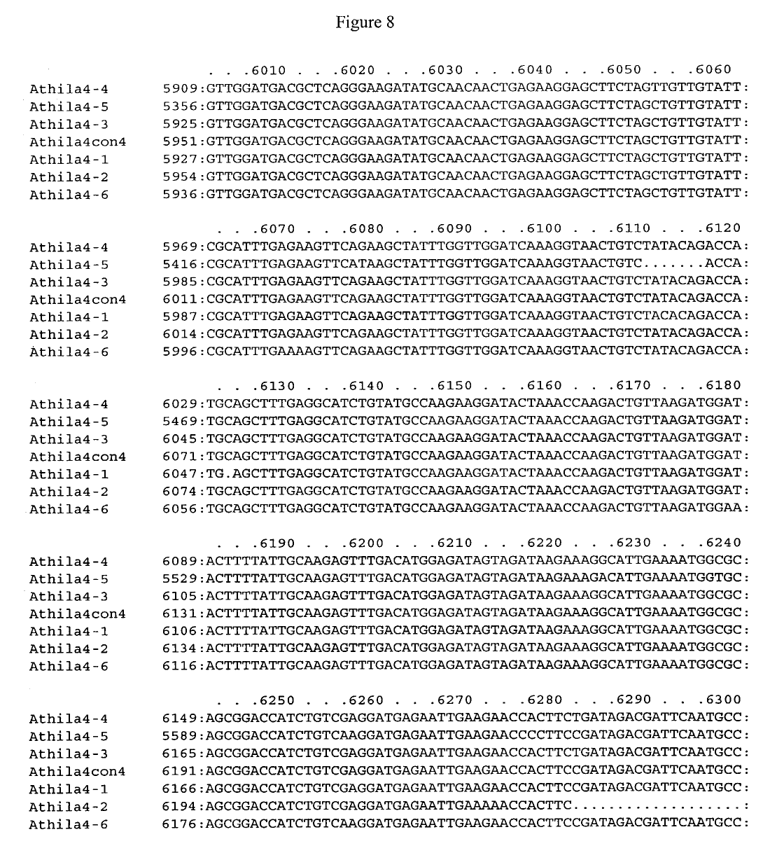

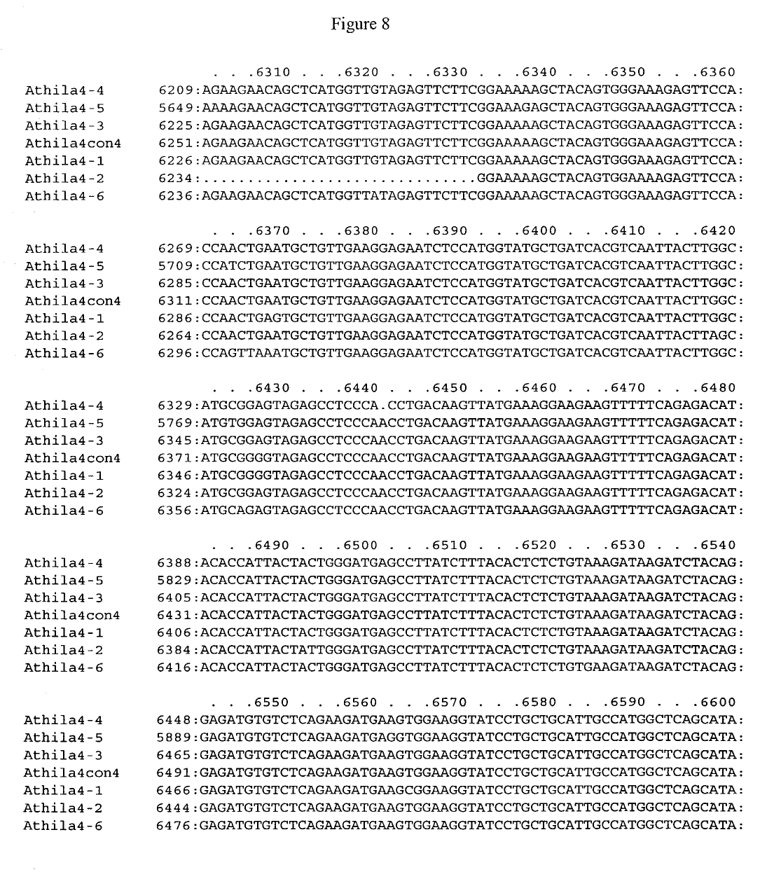

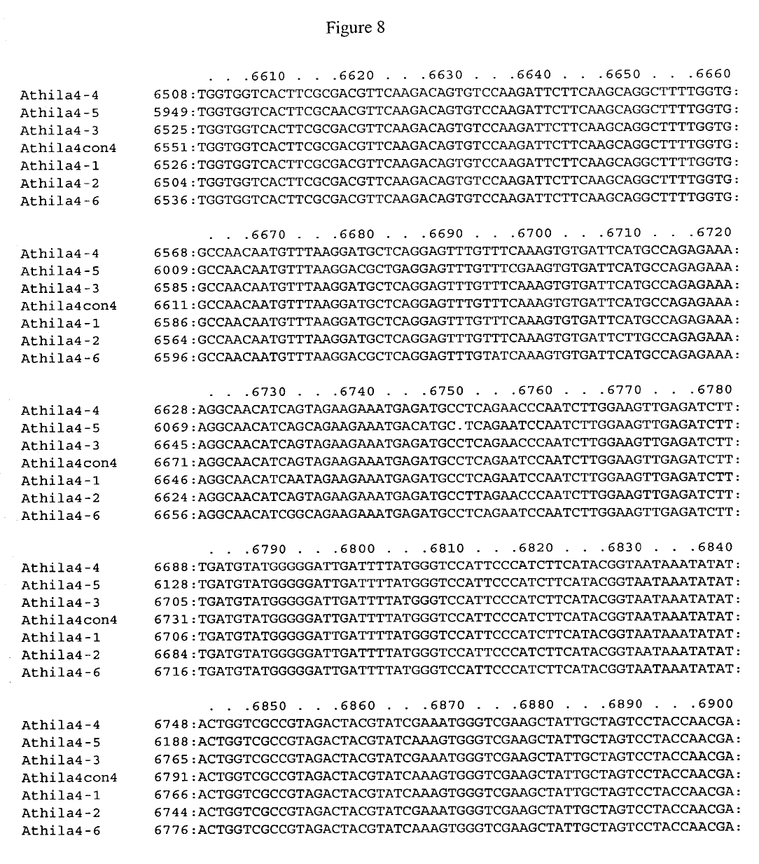

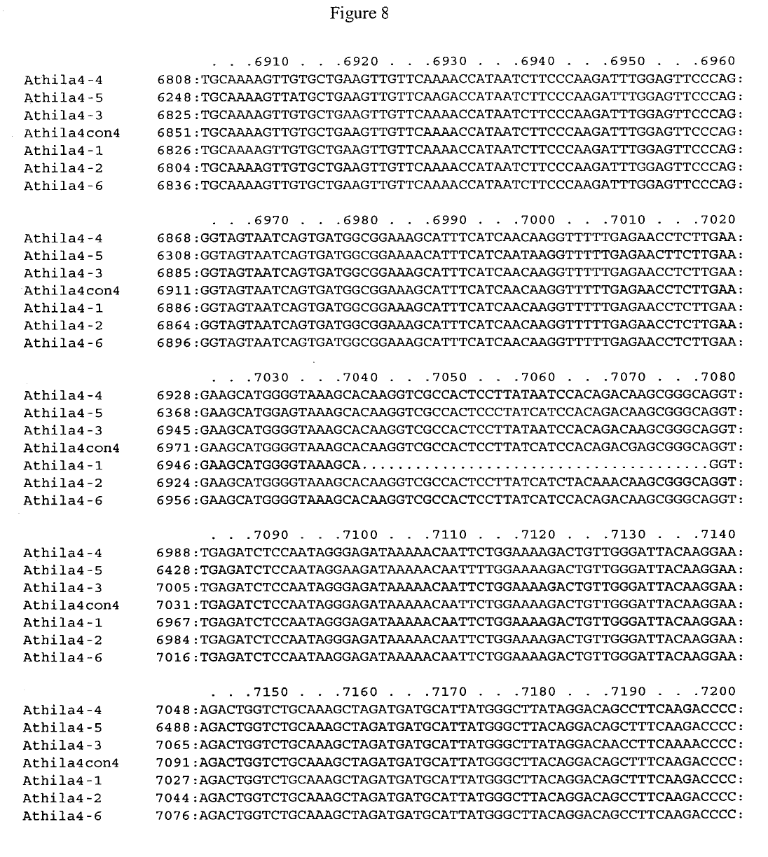

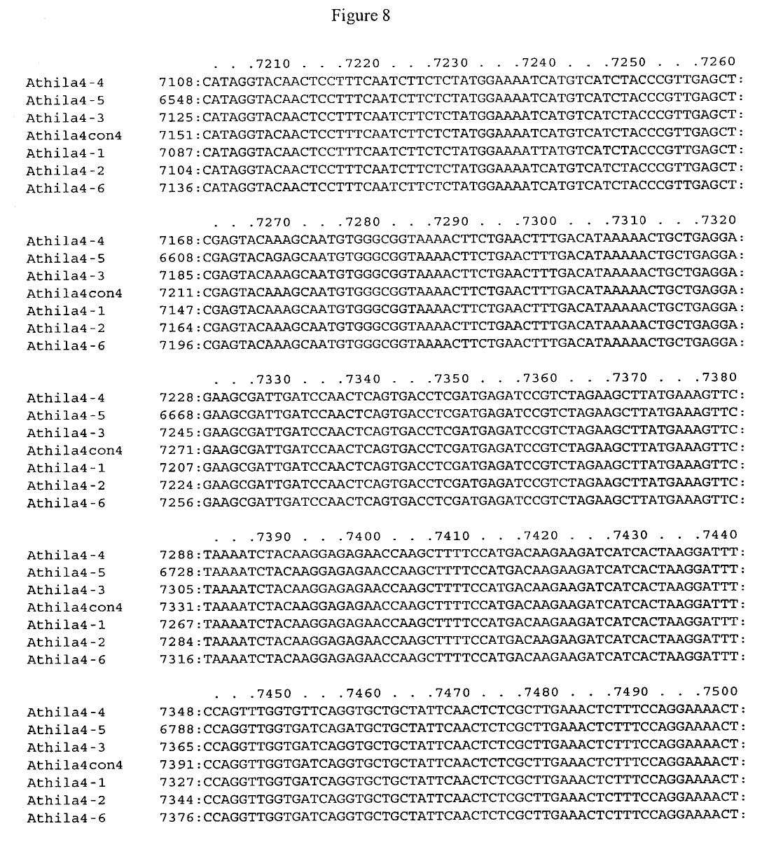

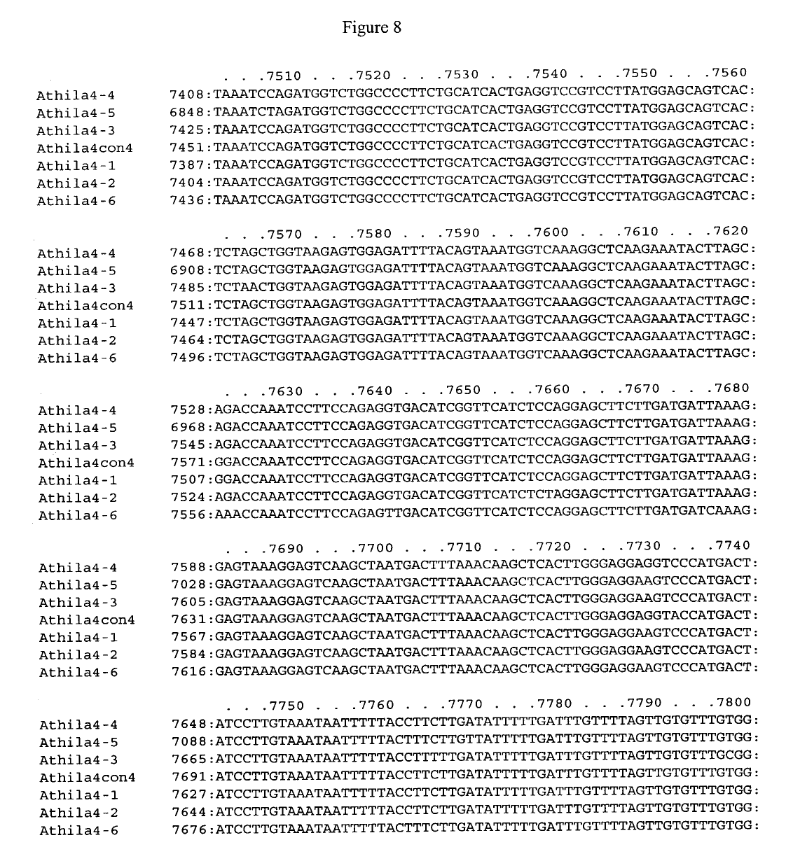

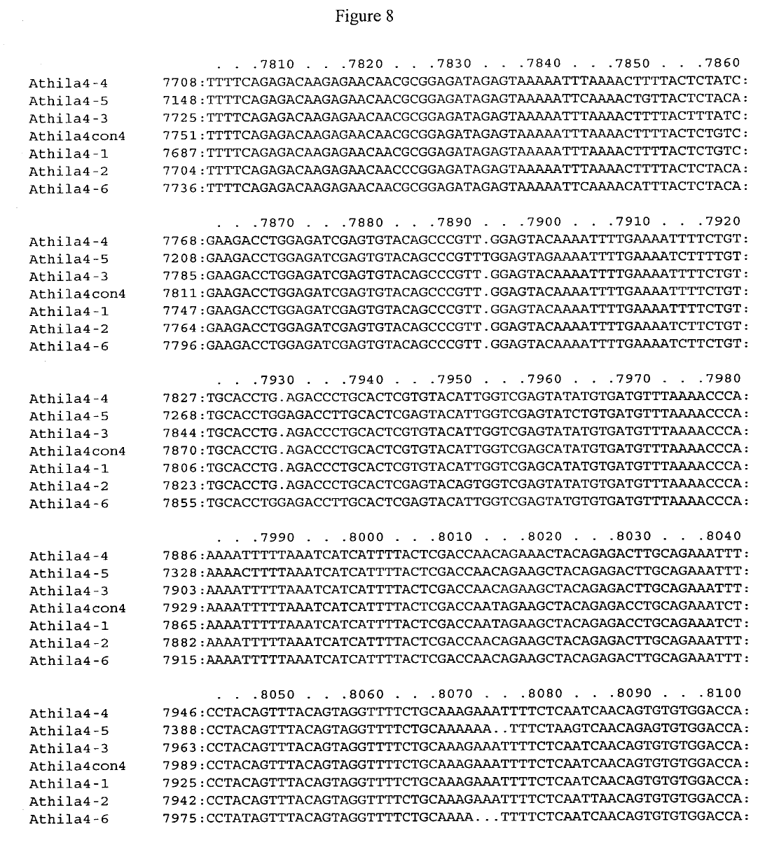

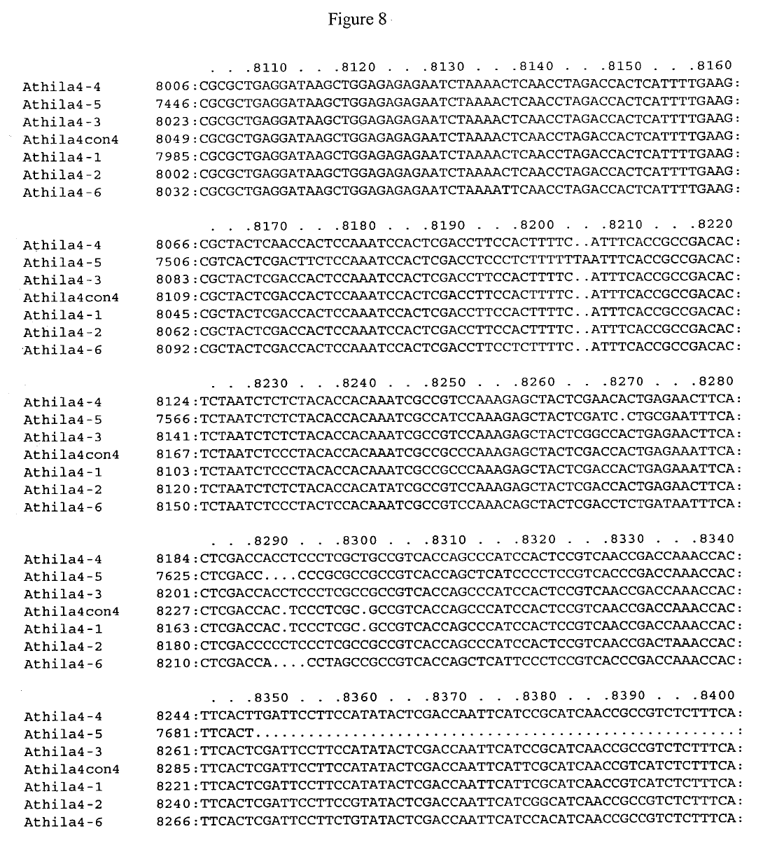

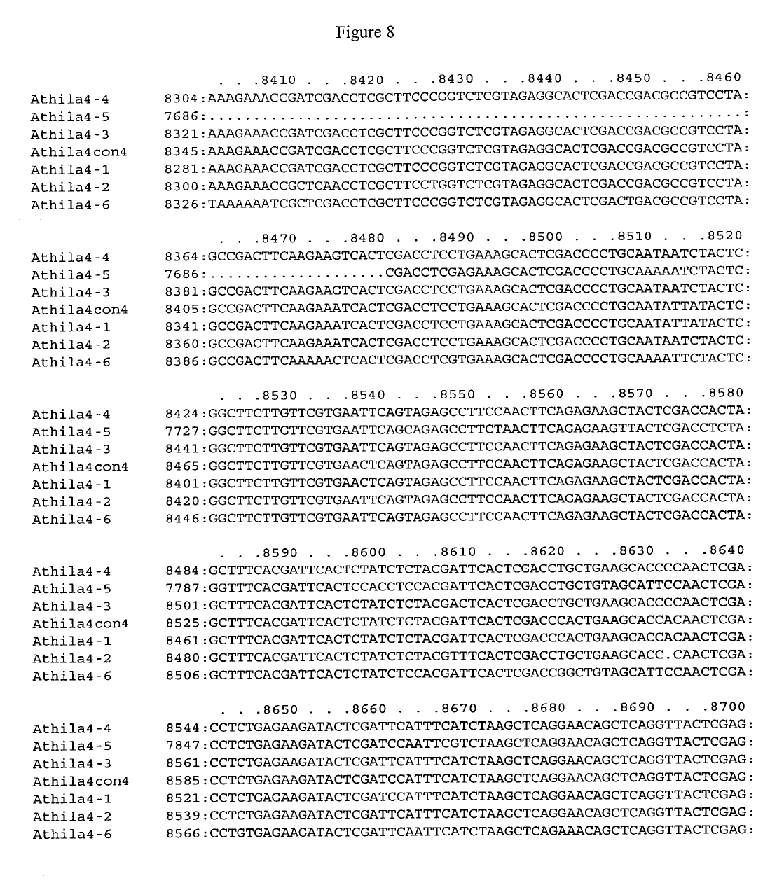

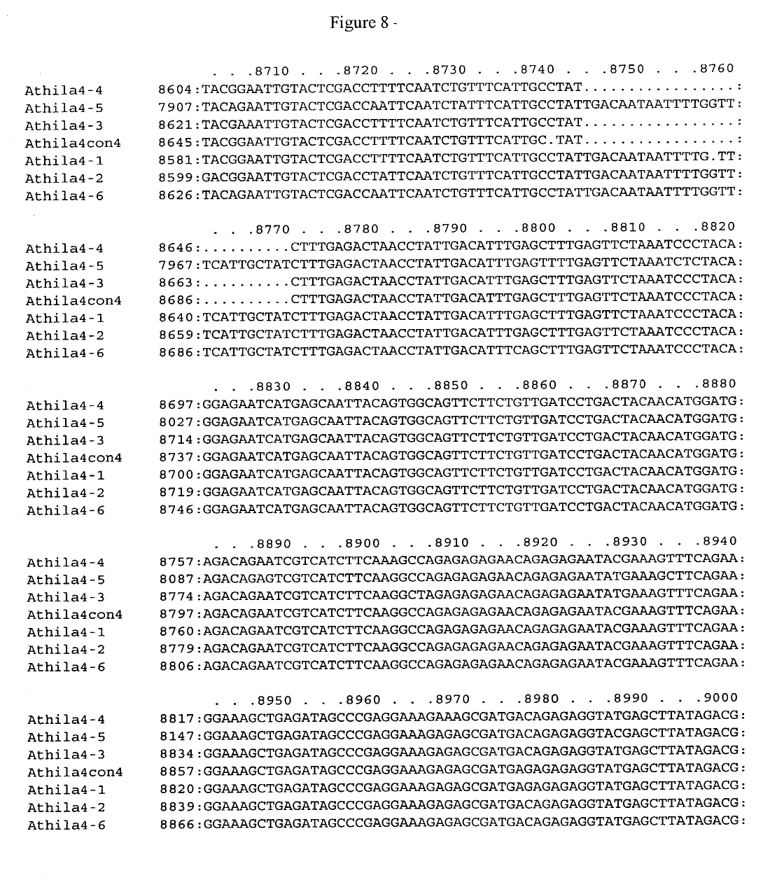

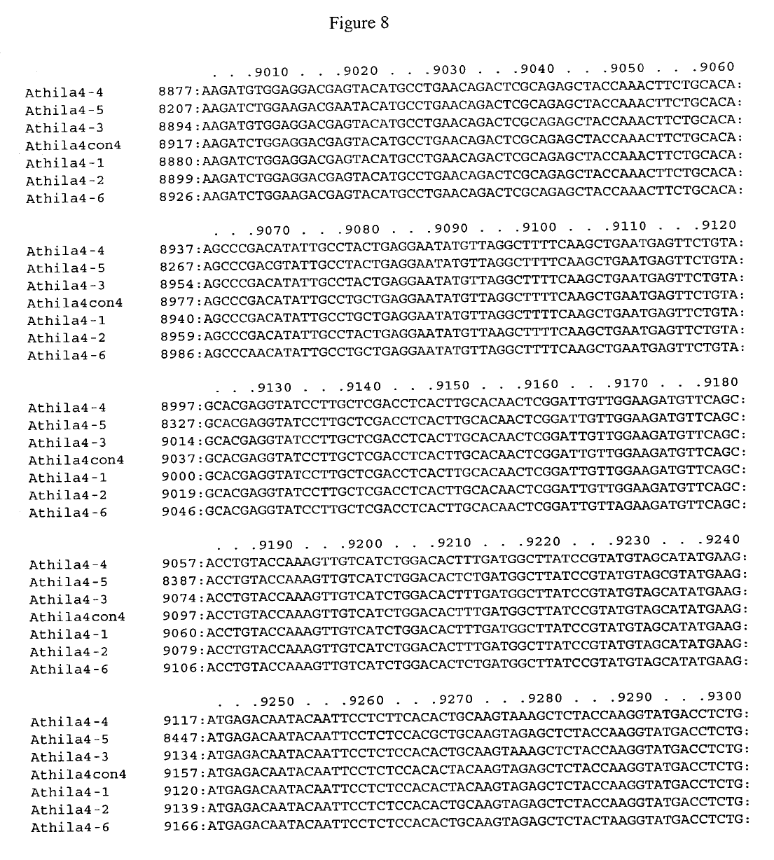

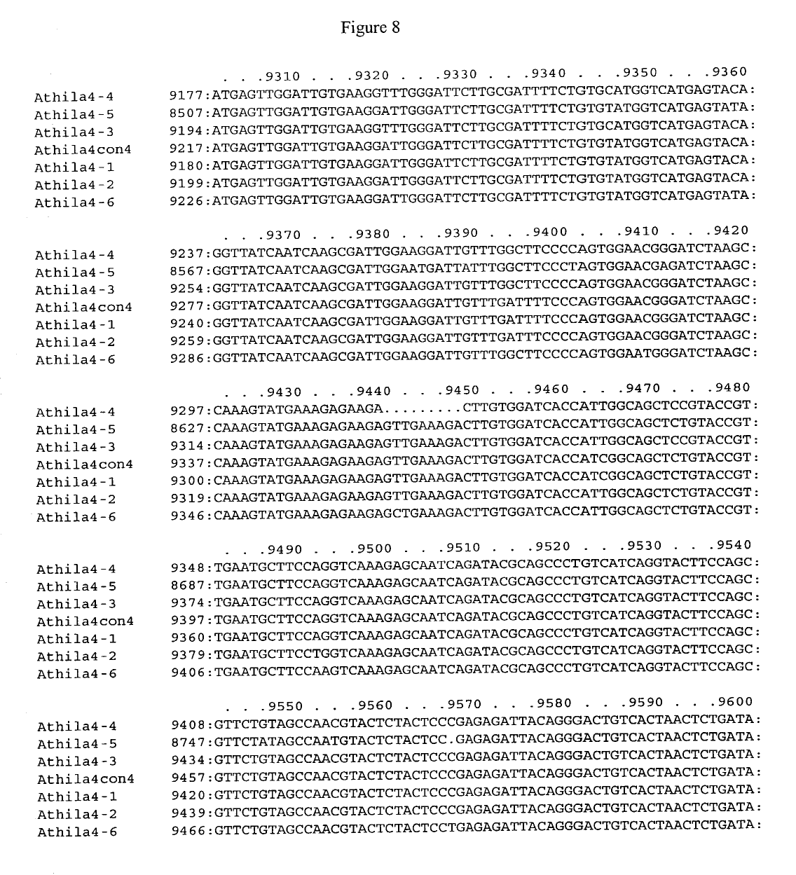

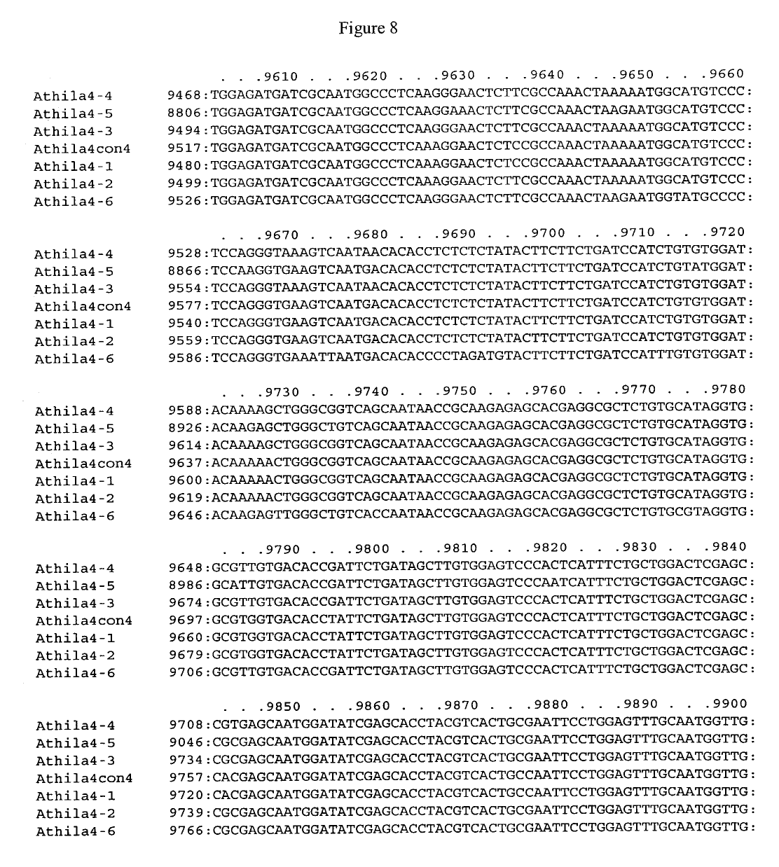

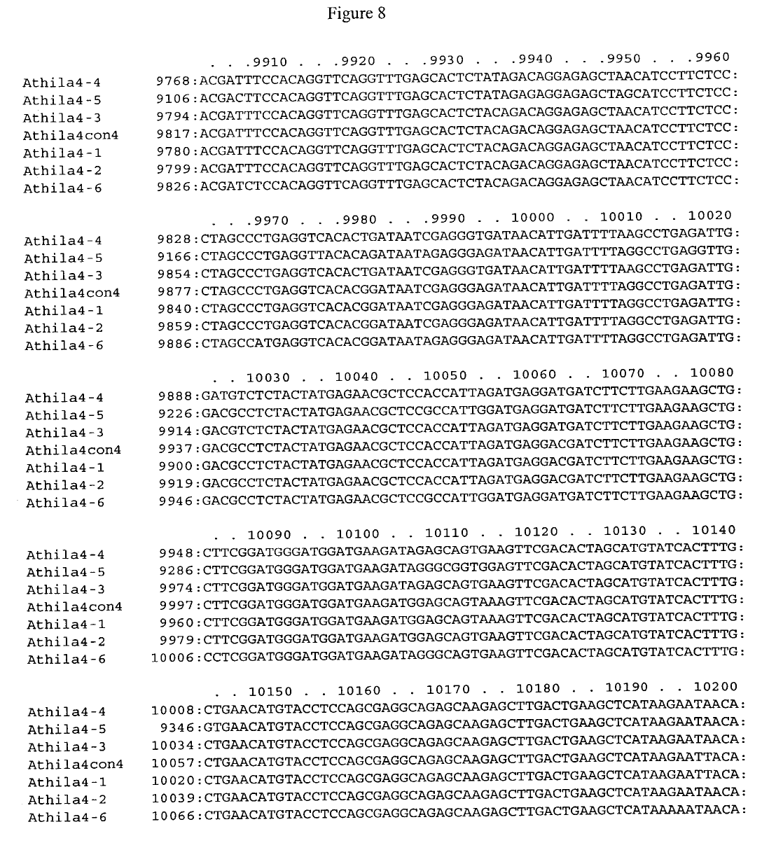

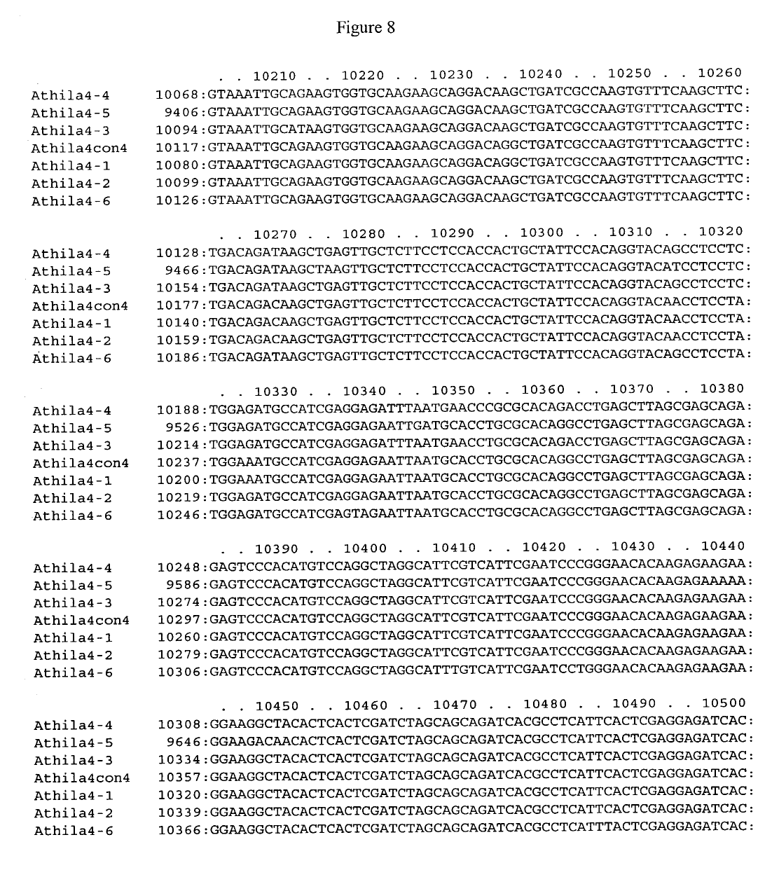

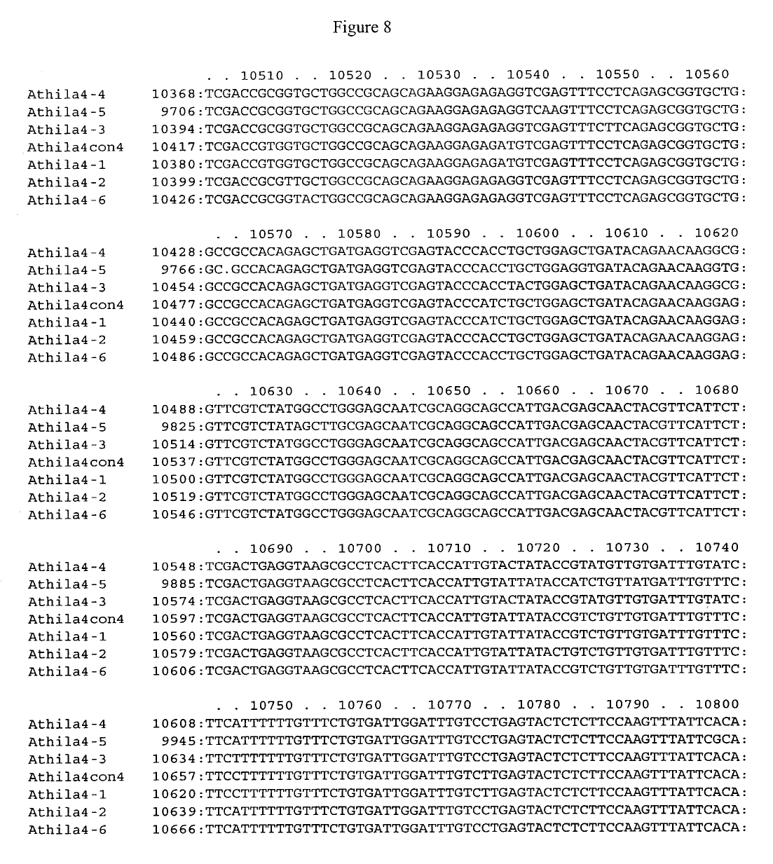

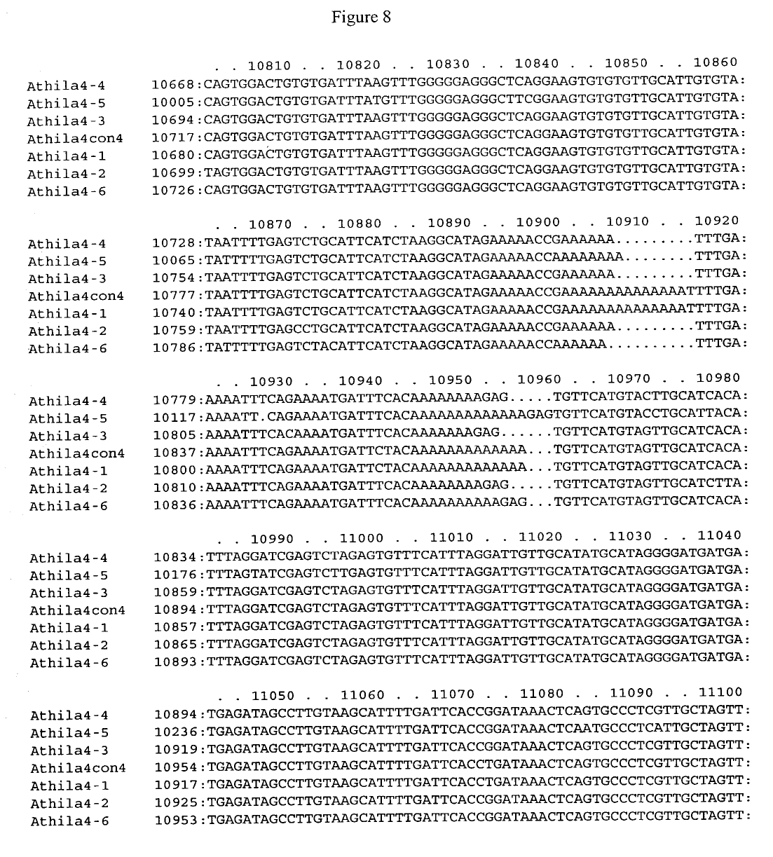

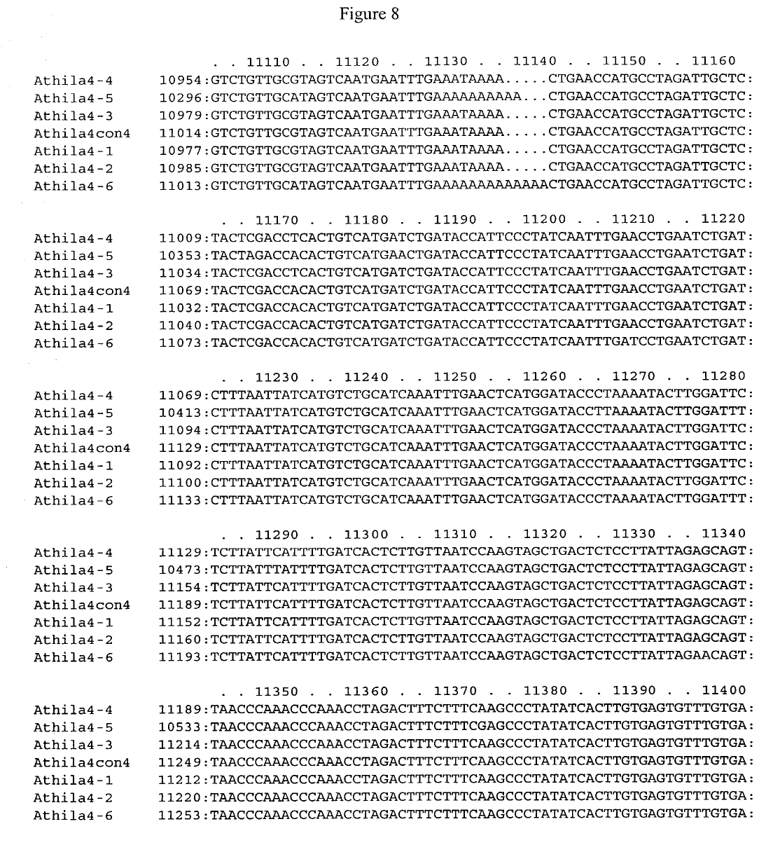

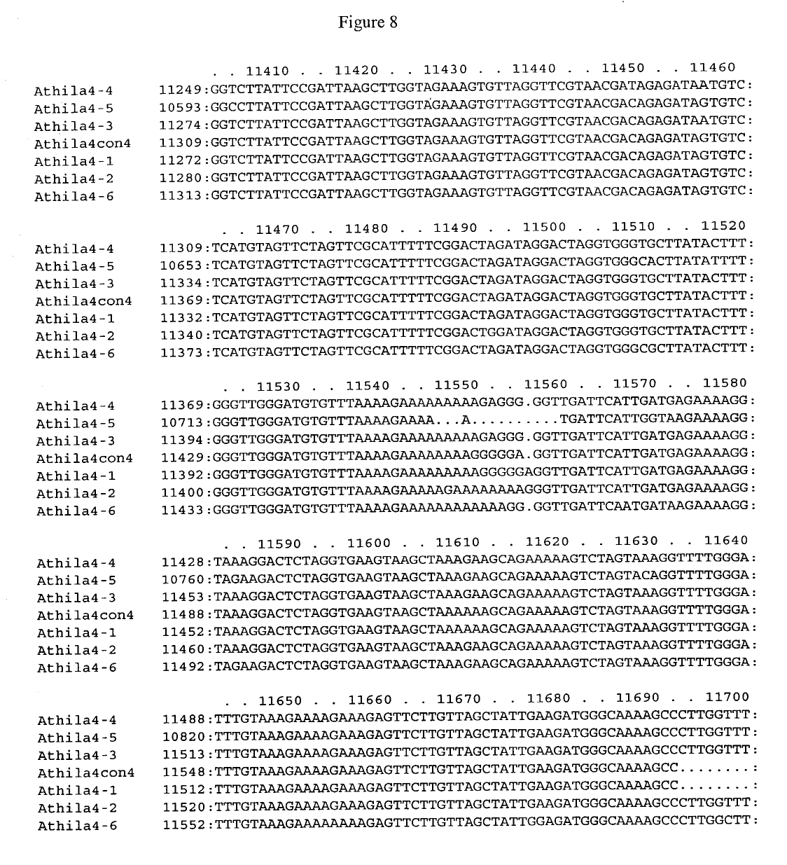

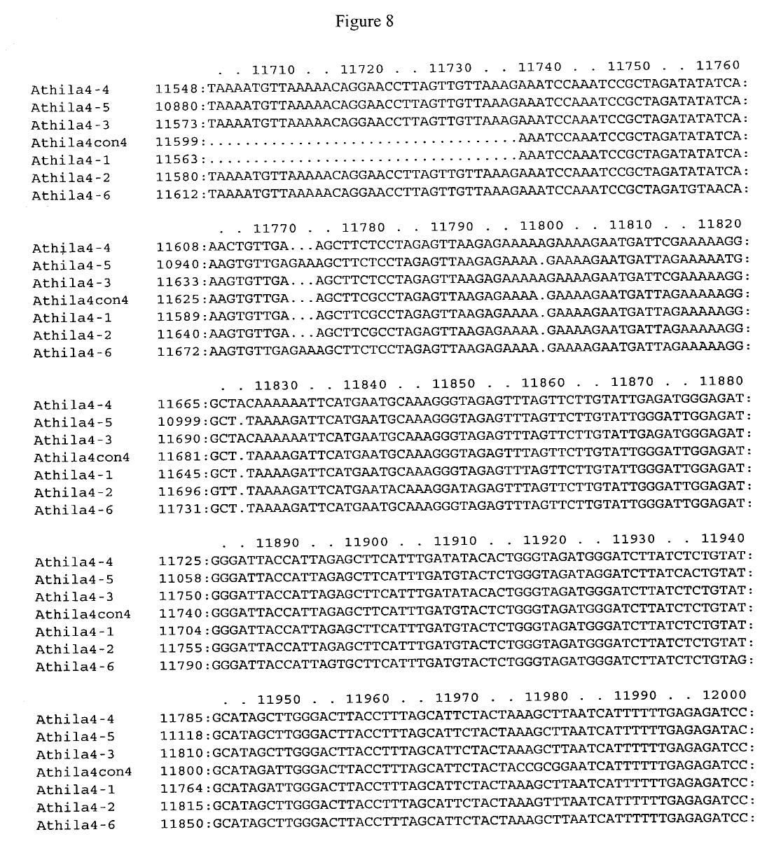

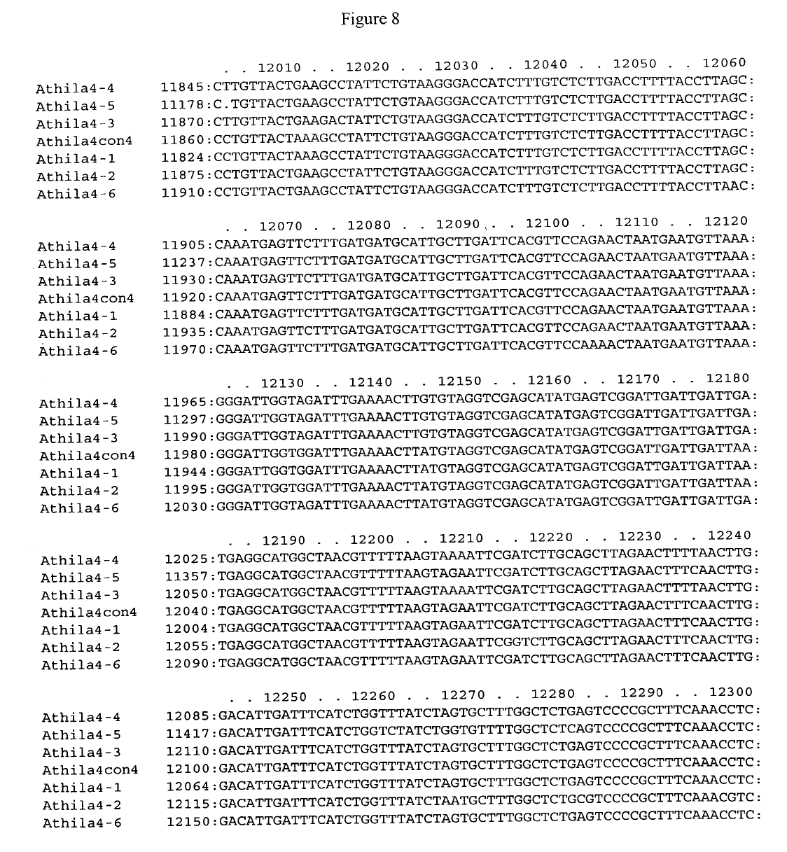

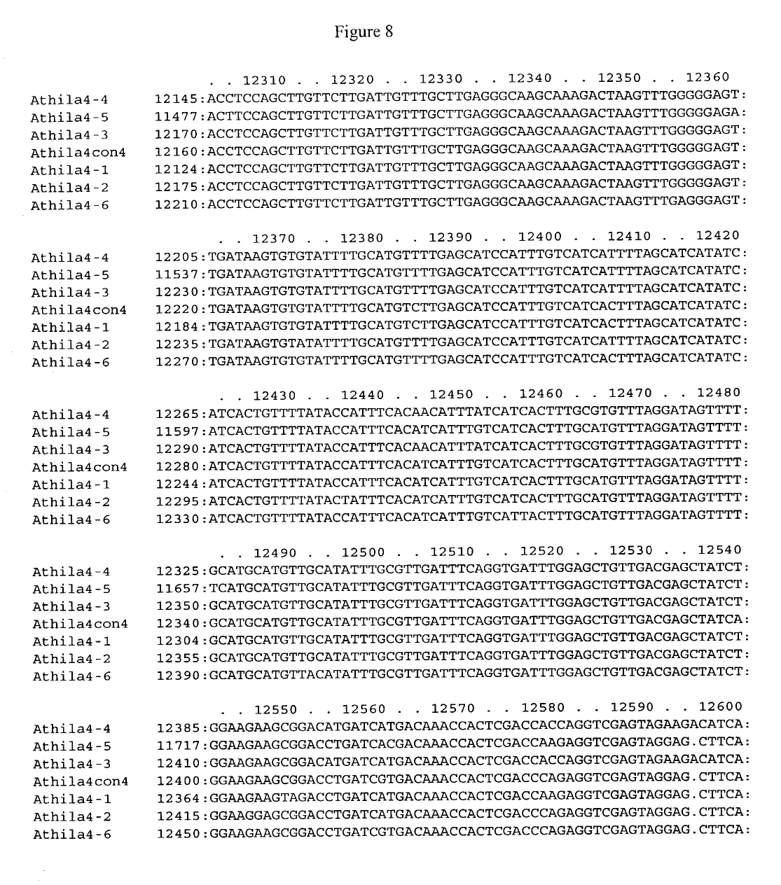

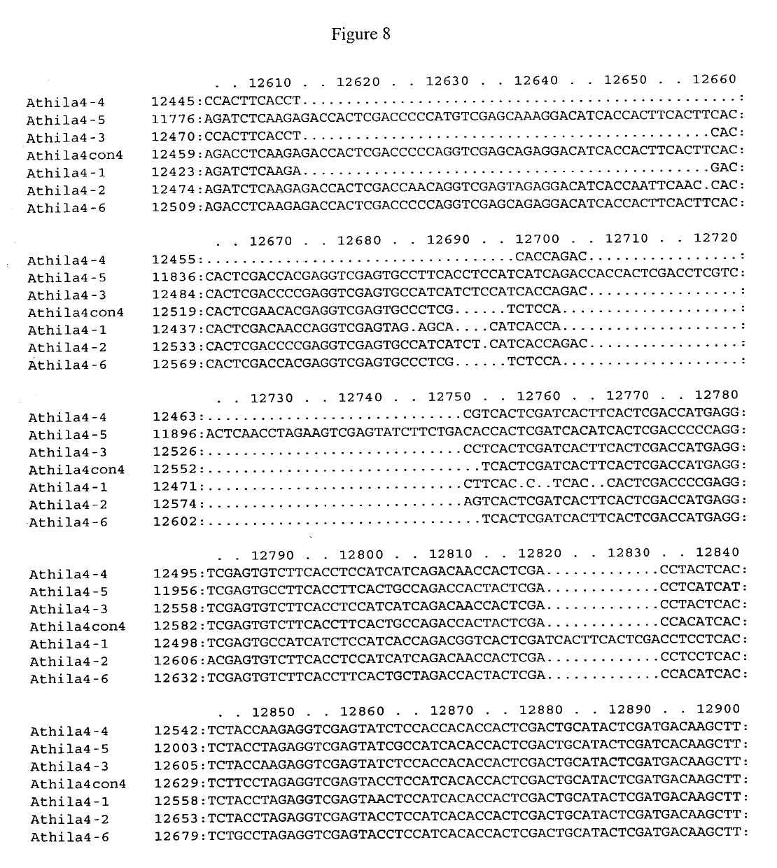

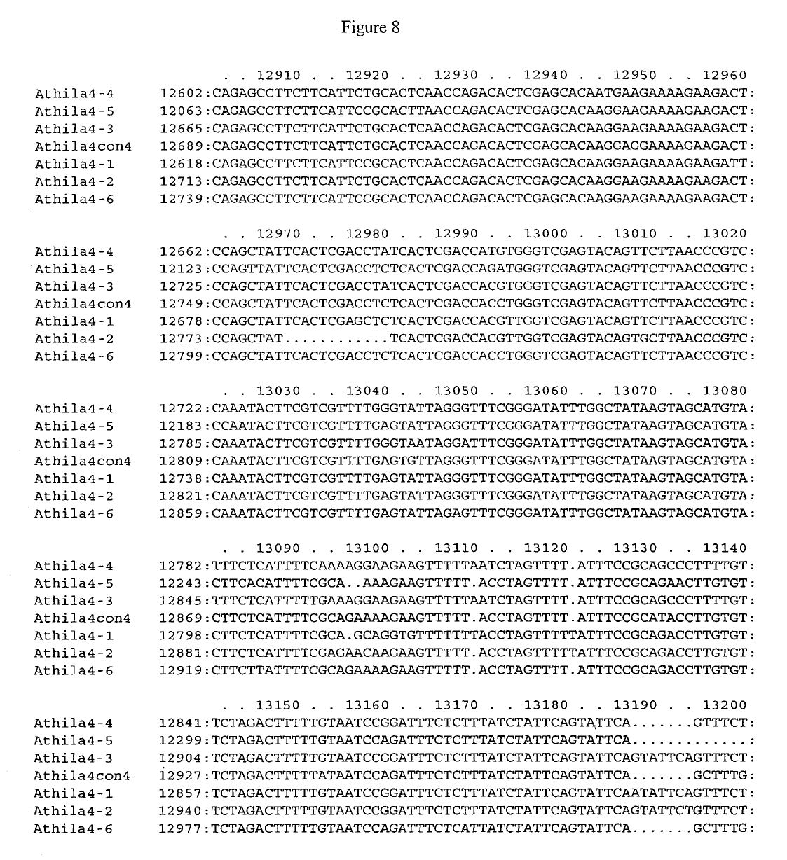

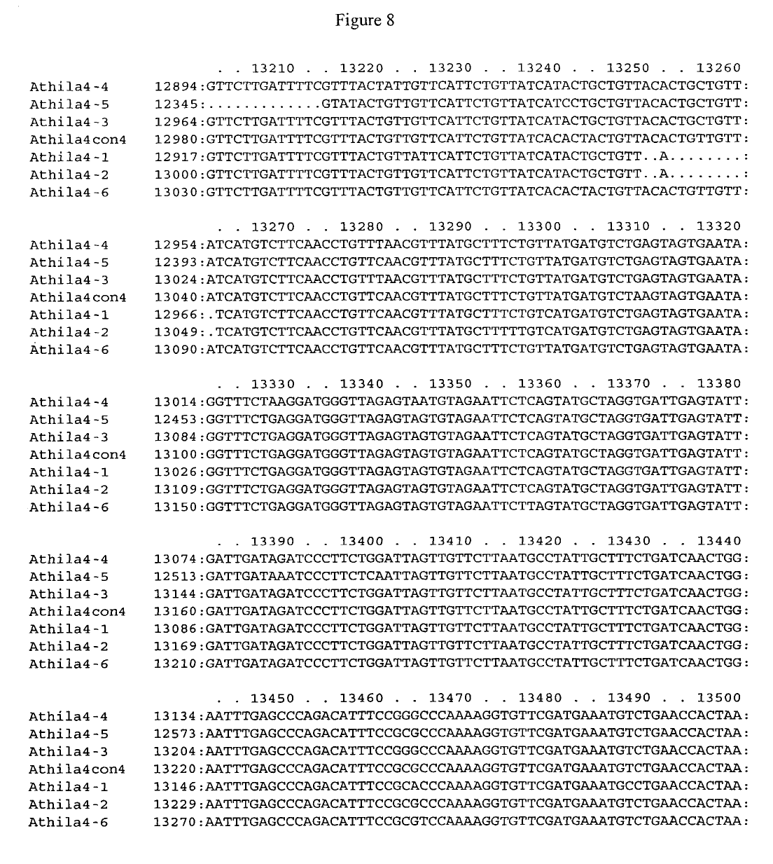

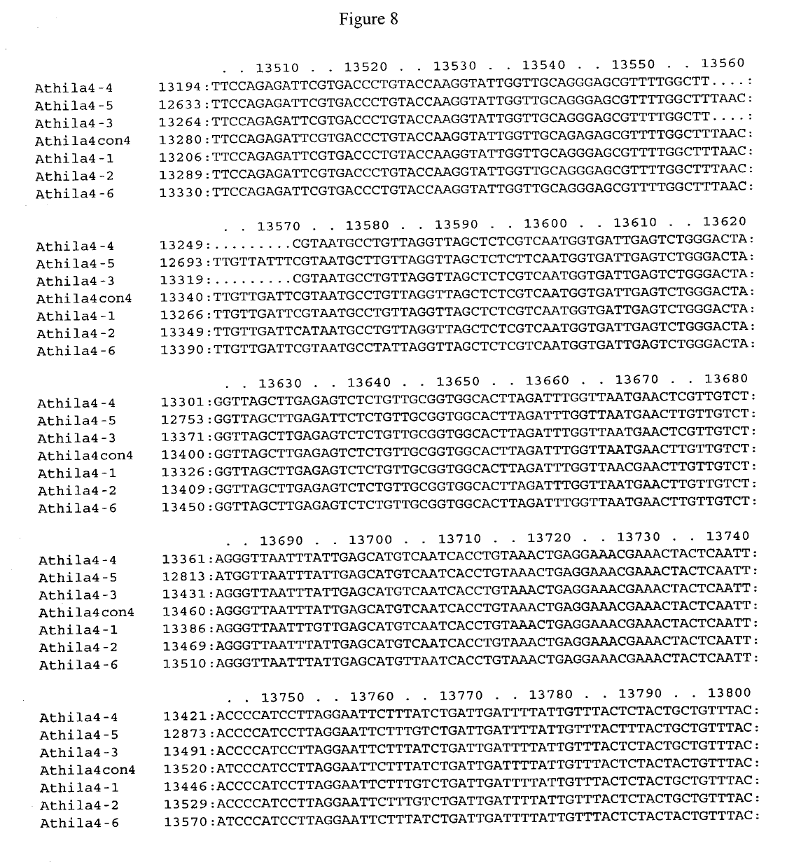

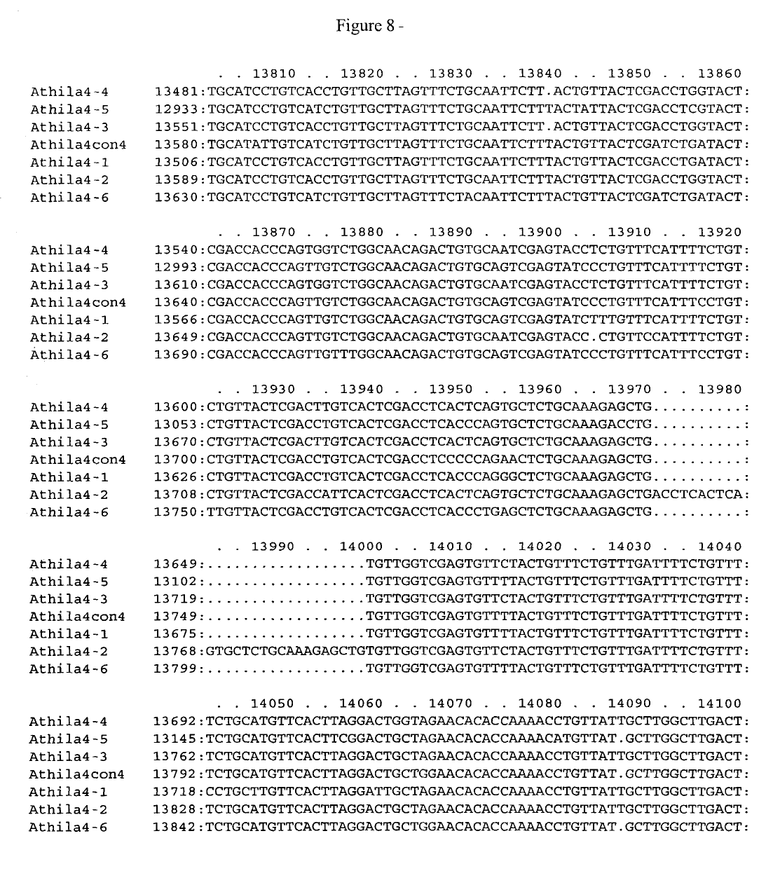

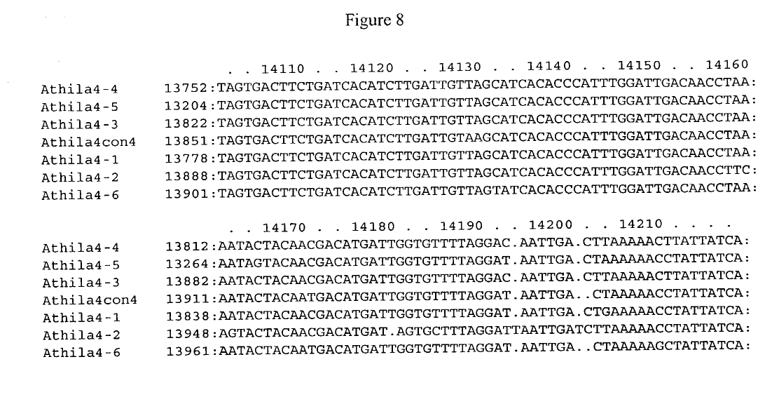

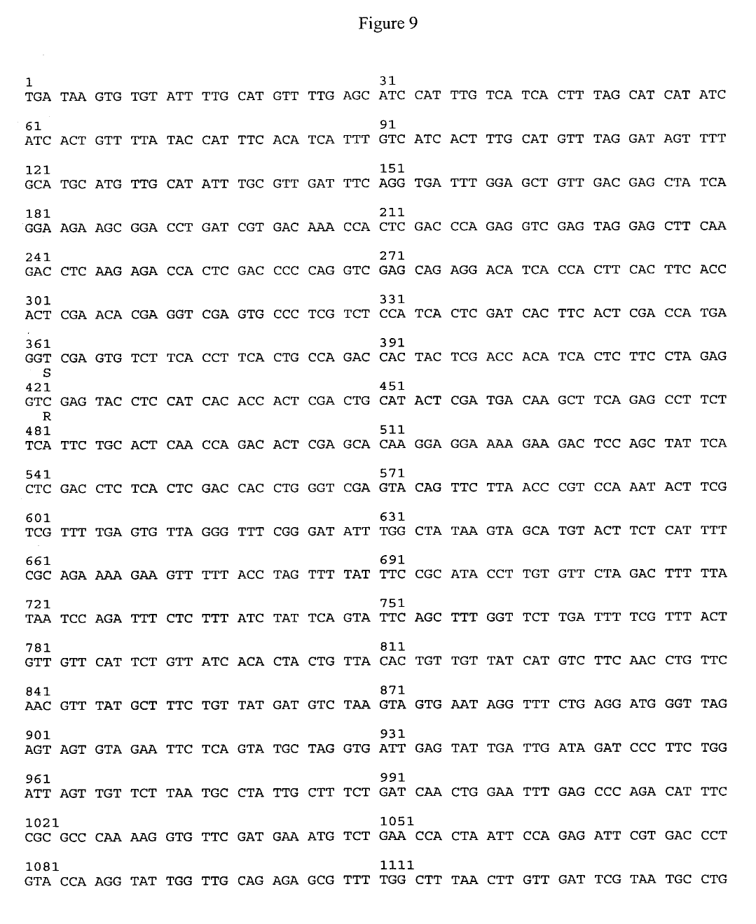

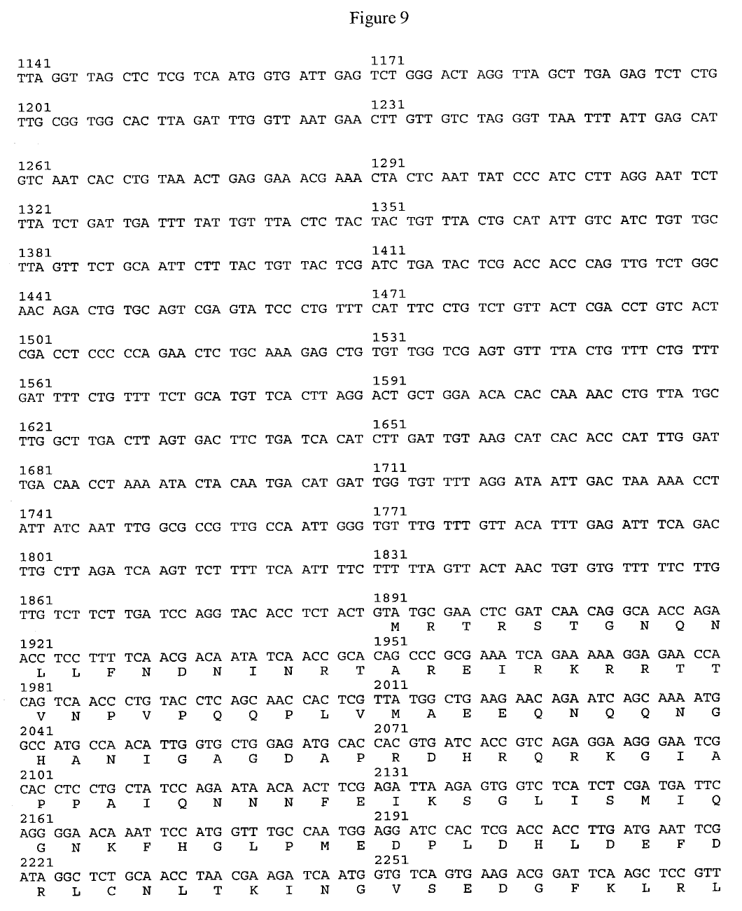

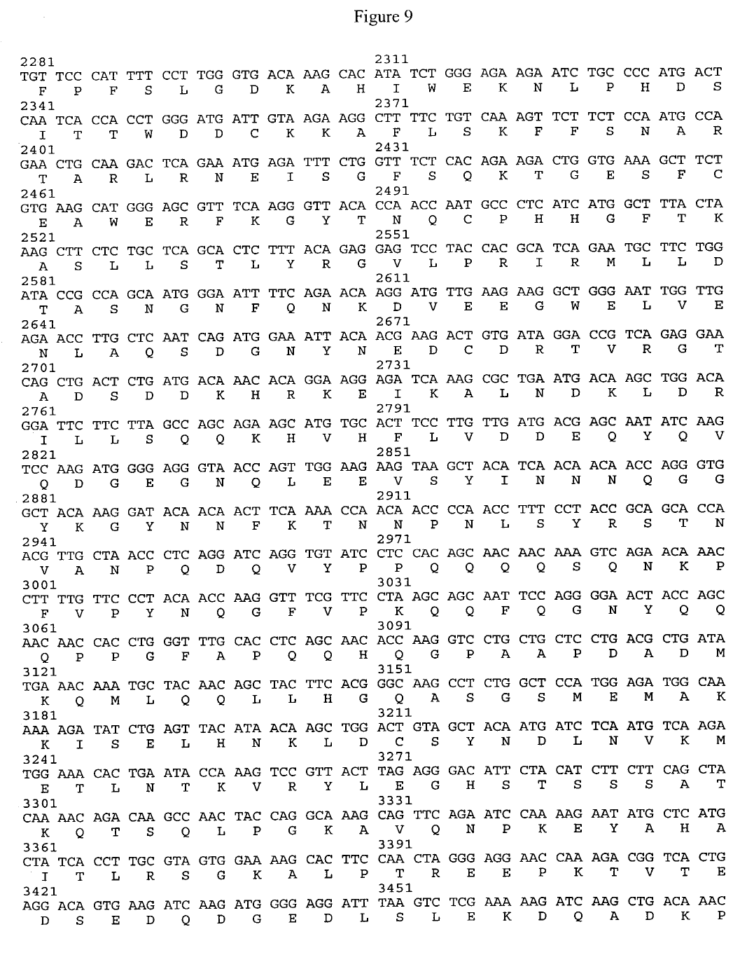

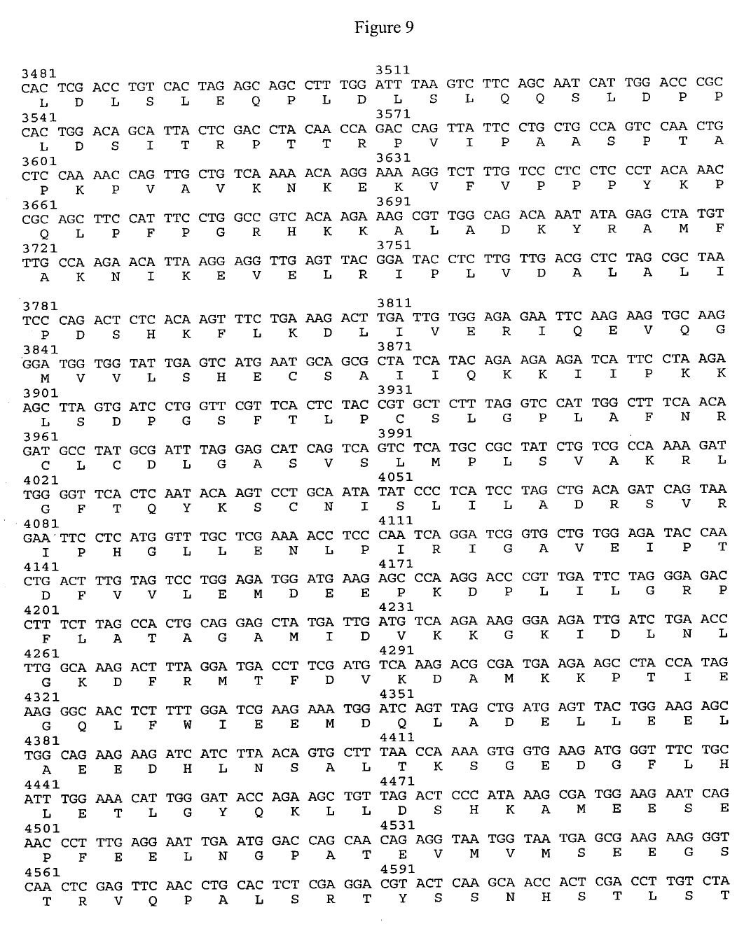

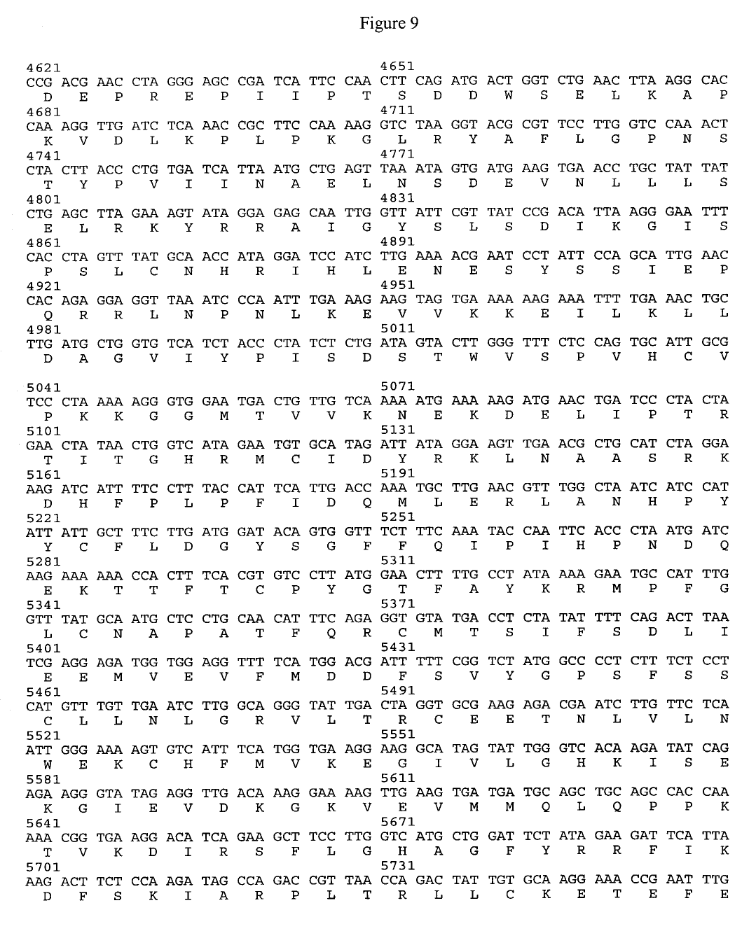

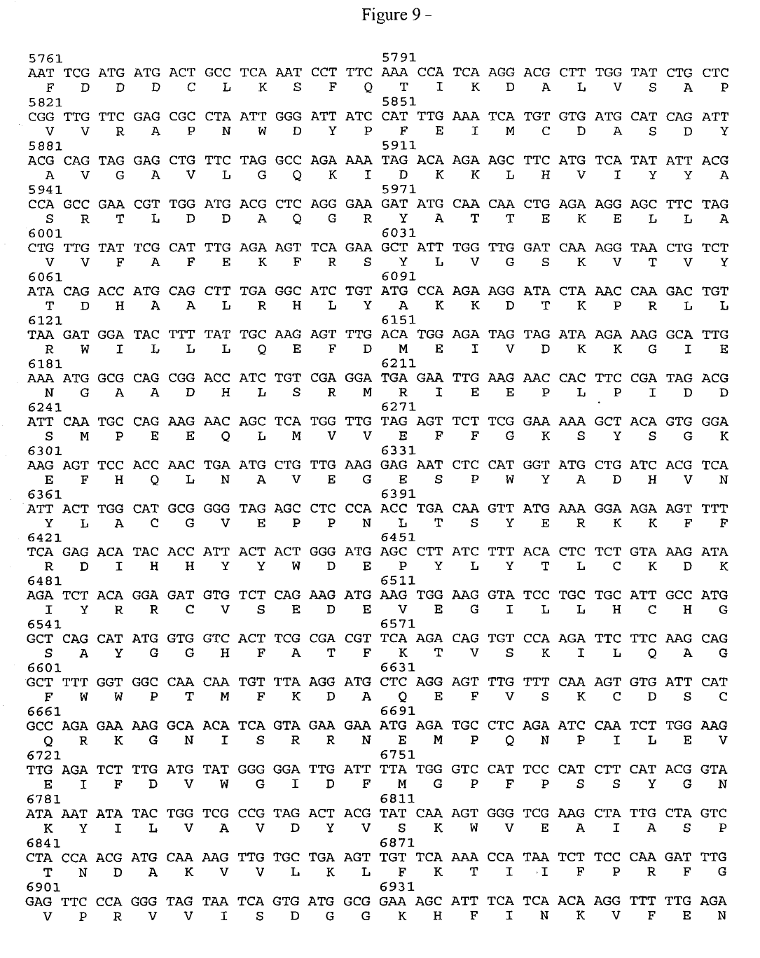

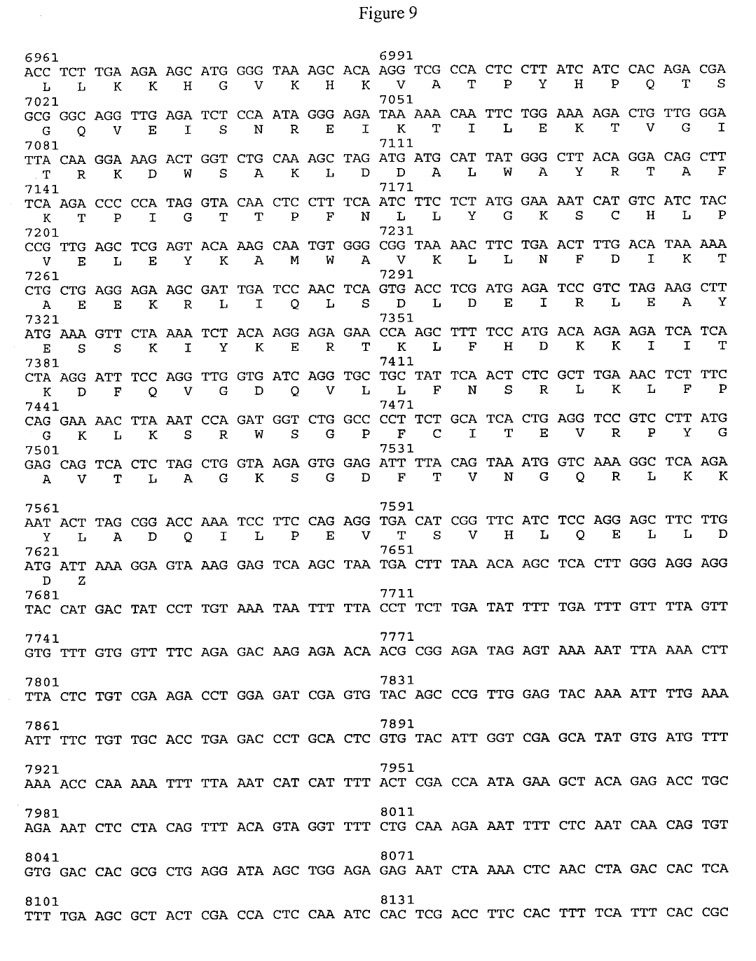

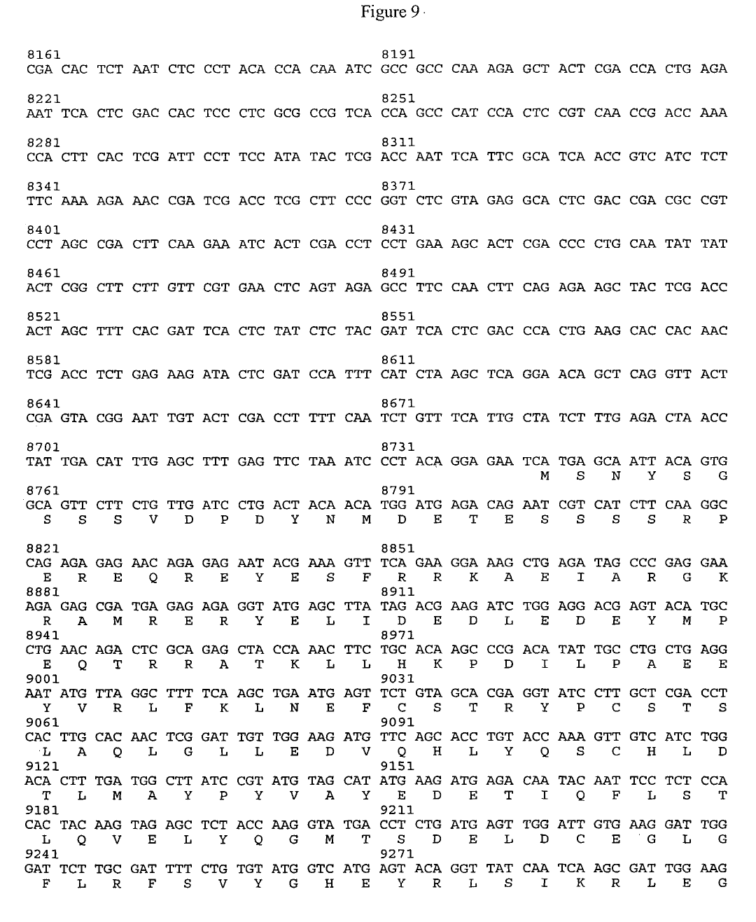

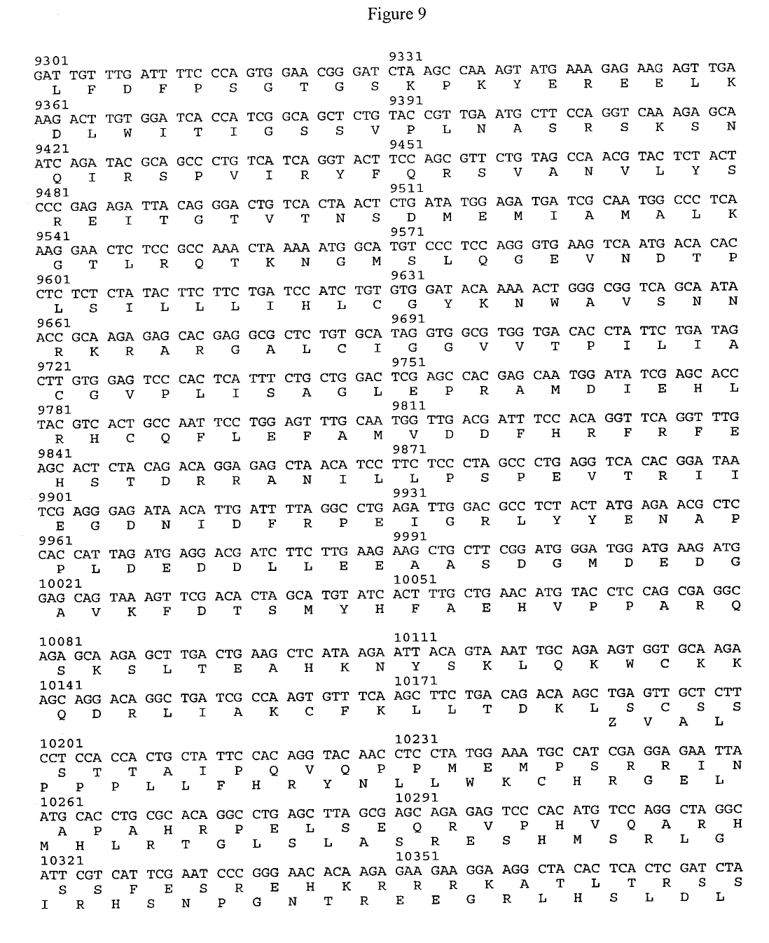

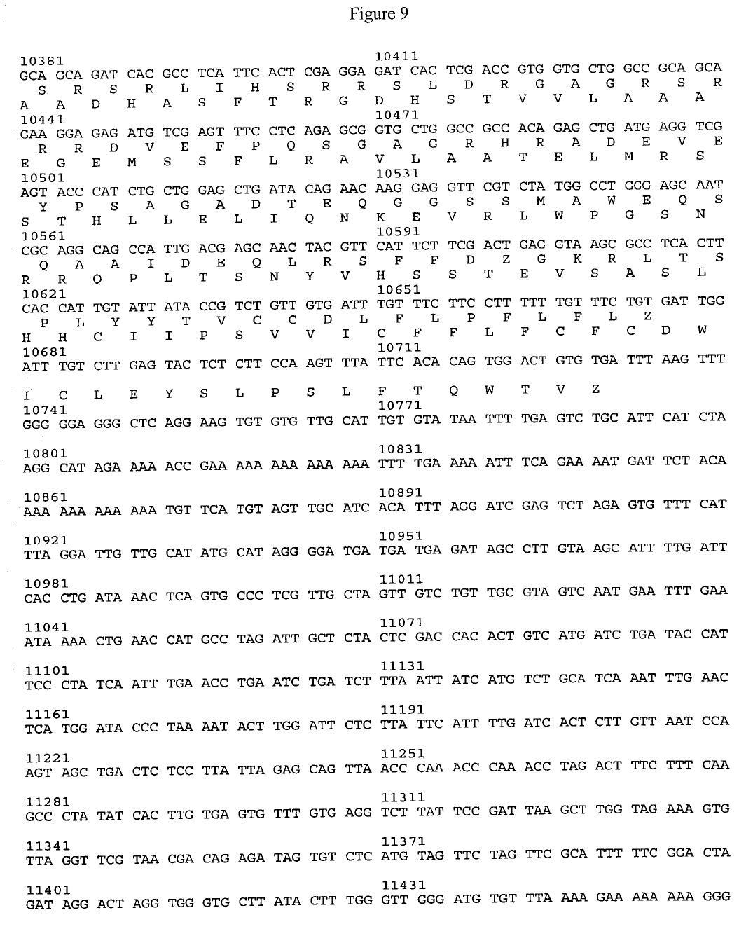

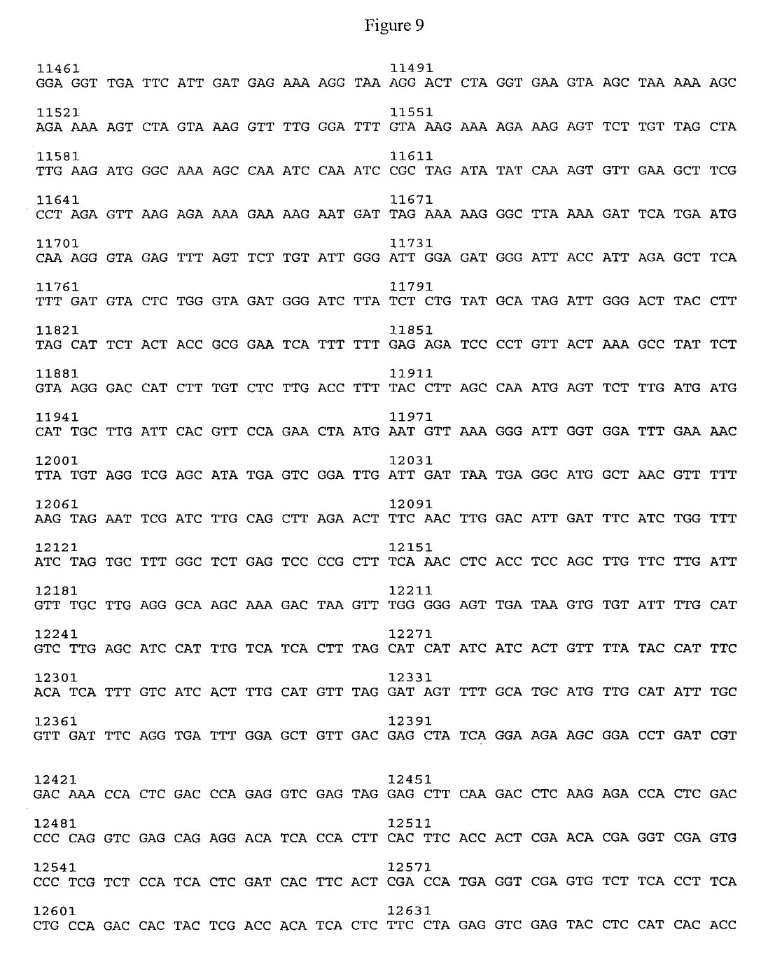

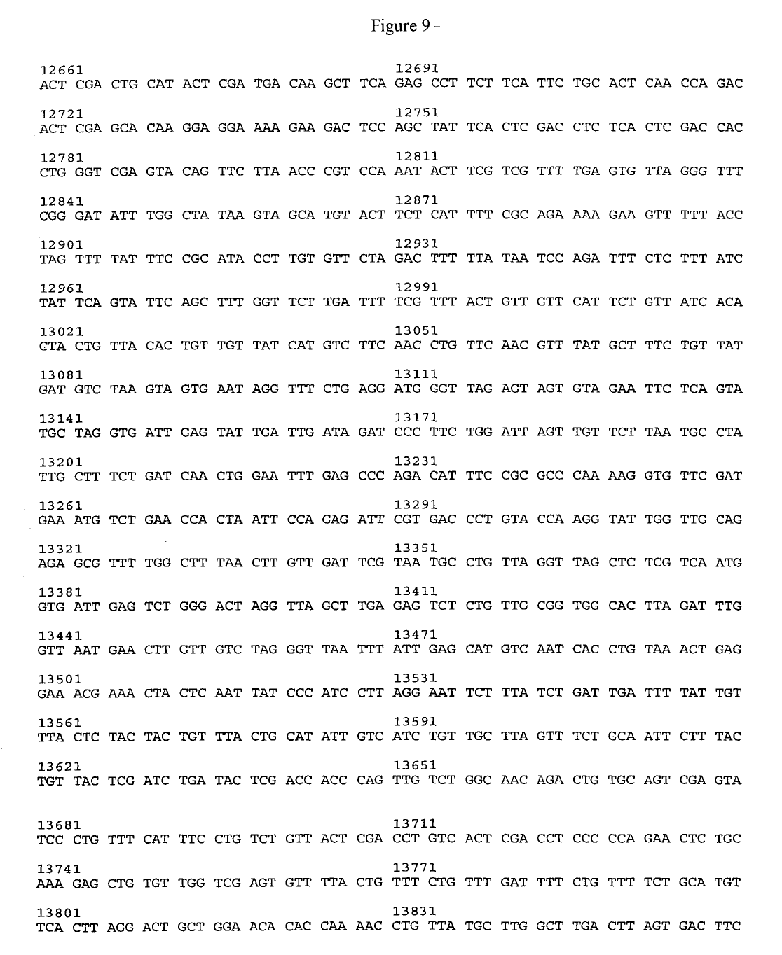

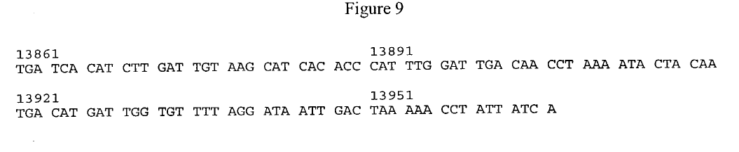

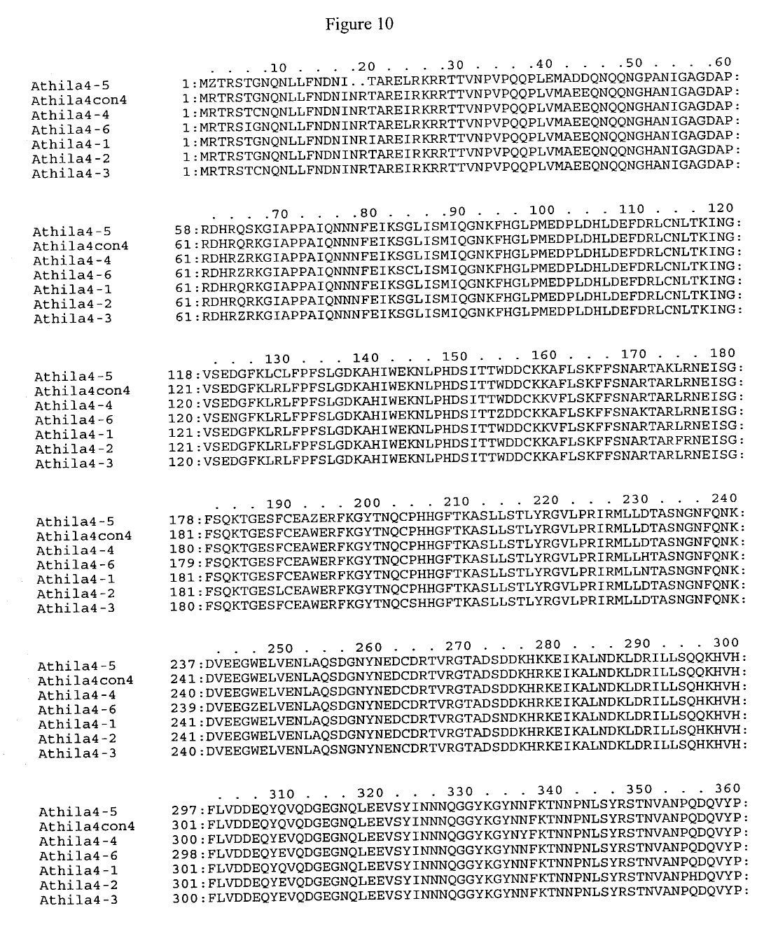

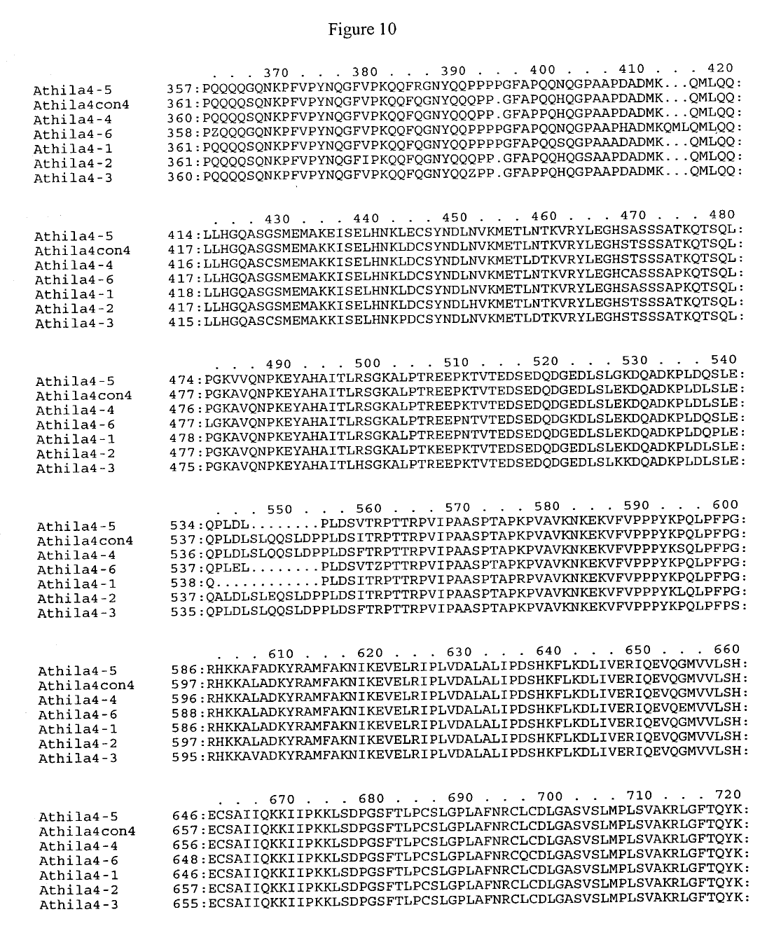

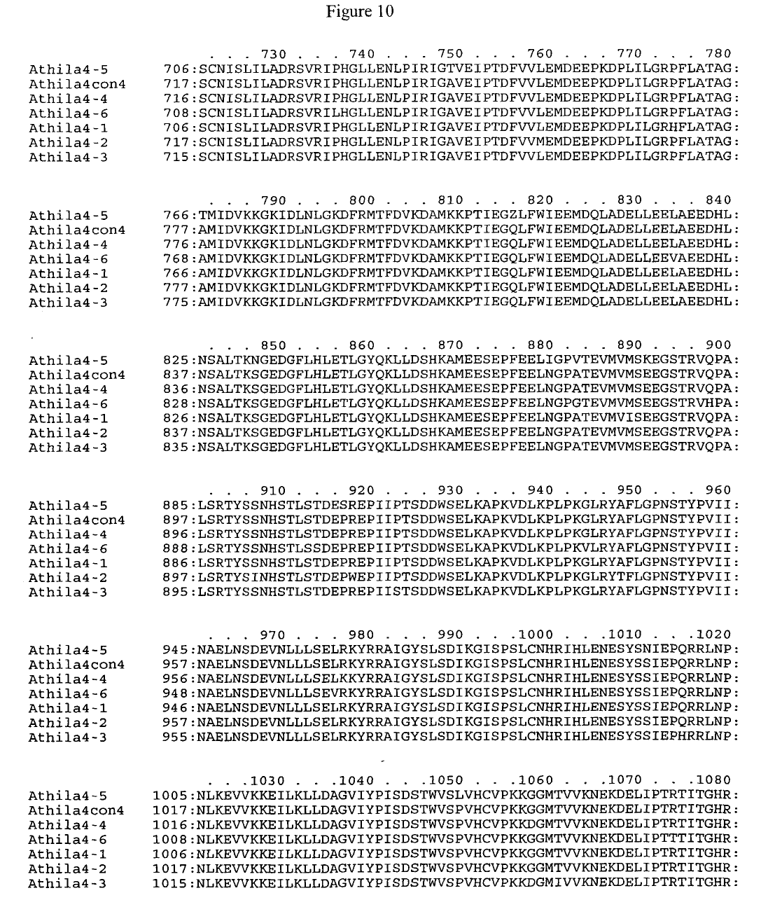

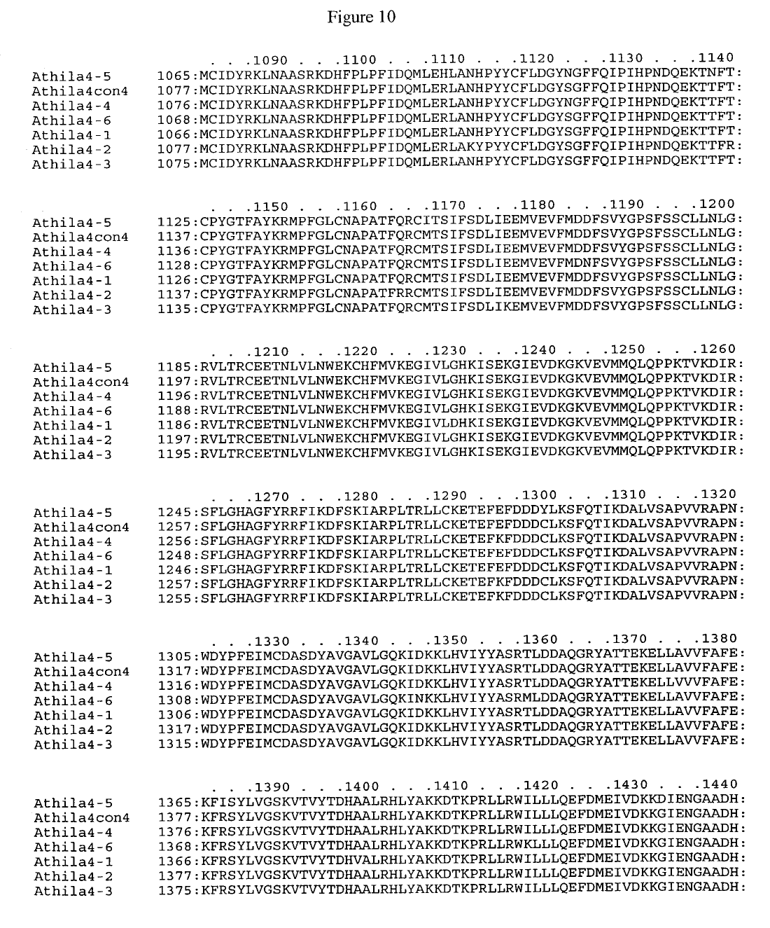

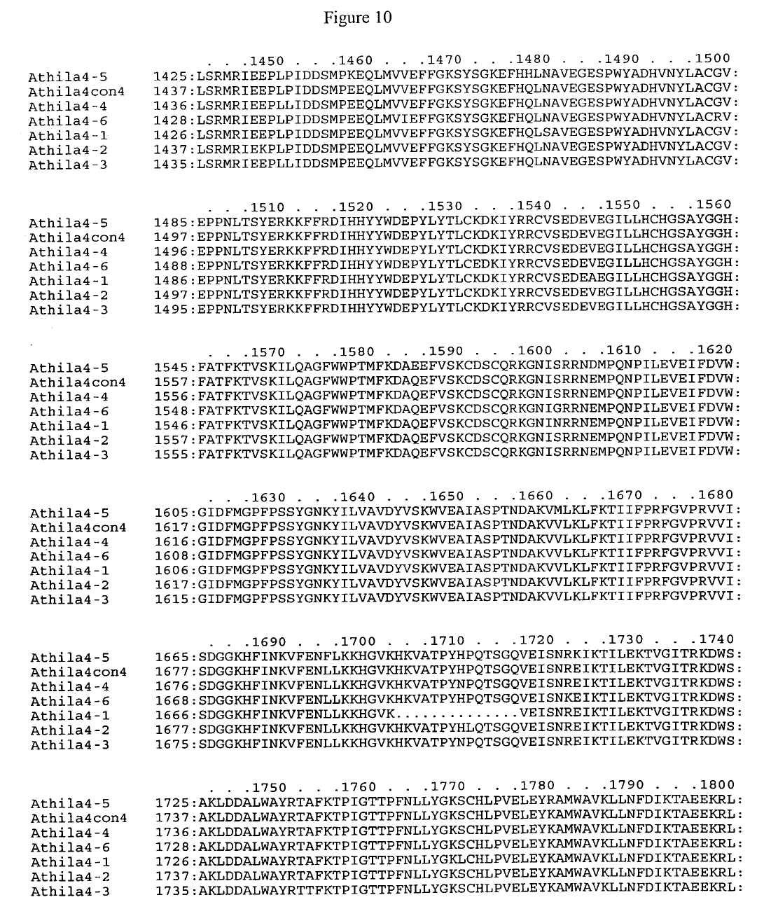

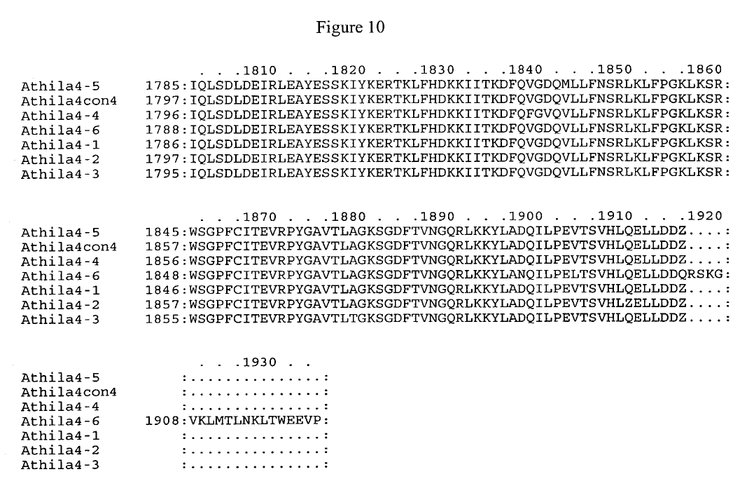

The invention provides isolated nucleic acids encoding polypeptides that have sequence similarity to polypeptides encoded by plant retroelements. The invention also provides isolated nucleic acids having cis-acting sequences that carry out functions associated with active retroelements. A consensus nucleotide sequence is provided in SEQ ID NO:122. SEQ ID NO:122 encodes, inter alia, a polypeptide having amino acid sequence SEQ ID NO:128 for a Gag/Pol polyprotein (approximate nucleotide positions 1891 to 7626, see FIGS. 8 and 9). Athila retroelement genomic sequences include Athila4-1 (SEQ ID NO:121), encoding, inter alia, amino acid sequence SEQ ID NO:135 for an Athila4-1 Gag/Pol polyprotein (see FIGS. 8 and 10). Other Athila retroelements include the Athila4-2 genome (SEQ ID NO:126) encoding, inter alia, an amino acid sequence (SEQ ID NO:136) for an Athila4-2 Gag/Pol polyprotein; the Athila4-3 genome (SEQ ID NO:125), encoding, inter alia, an amino acid sequence SEQ ID NO:137 for an Athila4-3 gag/pol polyprotein; the Athila4-4 genome (SEQ ID NO:123) encoding, inter alia, an amino acid sequence SEQ ID NO:133 for an Athila4-3 Gag/Pol polyprotein; the Athila4-5 genome (SEQ ID NO:124), encoding inter alia amino acid sequence SEQ ID NO:132 for an Athila4-5 Gag/Pol polyprotein; and the Athila4-6 genome (SEQ ID NO:127), encoding, inter alia, amino acid sequence SEQ ID NO:134 for an Athila4-6 Gag/Pol polyprotein (see FIGS. 8 and 10).

As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that encode non-PAPSS1 proteins). The term “isolated” as used herein with respect to nucleic acids also includes any non-naturally-occurring nucleic acid sequence since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A consensus nucleotide sequence can be identified by aligning a number of nucleic acid sequences and identifying the most common nucleotide at each position. For example, (1) aligning the nucleotide sequences set forth in SEQ ID NO:121 and 123 through 127 and (2) determining the most common nucleotide at each position will give the nucleotide sequence of SEQ ID NO:122. A software program such as ClustalX (see, Thompson et al. (1997) Nucl. Acids Res. 24: 4876-4882) can be used to align multiple sequences.

Functional elements of the invention include the following:

5′ LTR at about consensus positions 1 to 1747;

promoter at about consensus positions 1 to 385;

LTR end sequences at about consensus positions 1 to 40;

LTR end sequences at about consensus positions 1708 to 1747;

PBS at about consensus positions 1751 to 1763;

gag nucleic acids at about consensus positions 1893 to 3575;

PR nucleic acids at about consensus positions 3576 to 4556;

RT nucleic acids at about consensus positions 4602 to 6314;

IN nucleic acids at about consensus positions 6315 to 7625;

env nucleic acids at about consensus position to 10600;

env nucleic acid at about consensus positions 8745 to 10673;

env nucleic acid at about consensus positions 8745 to 10728;

PPT1 at about positions 12205 to 12218;

PPT2 at about positions 10738 to 10747;

non-coding region at about positions 10729 to 12219;

non-coding region at about positions 7626 to 8744;

splice site acceptor at about positions 8736 to 8739;

3′ LTR at about consensus positions 12220 to 13966; and

transcript termination site at about positions 12963 to 12993.

The function of a 5′ LTR is to provide promoter, polyadenylation, enhancer, and/or silencer function. The LTR also provides integration sequences (LTR end sequences) that are DNA sequences recognized by integrase and used by integrase for inserting a retroviral cDNA into the genome of a host. As provided herein, LTR end sequences also can be used to insert heterologous nucleic acids into the genome of selected eukaryotic cells. A suitable 5′ LTR nucleic acid resides at about position 1 to about position 1747 of (SEQ ID NO:122).

A promoter is found within the 5′ LTR at about position 1 to about position 385 of SEQ ID NO:122.

Functions related to integration are found at approximately position 1 to approximately position 40 of SEQ ID NO:122 and at about position 1708 to about position 1747 of SEQ ID NO:122.

A PBS can provide a recognition and binding site for the 3′ end of aspartic acid tRNA from A. thaliana. The PBS can prime minus strand DNA synthesis in A. thaliana. A PBS is found at about position 1751 to about position 1763 of SEQ ID NO:122.

A gag nucleic acid can have an open reading frame for a Gag polypeptide that can be processed into a retroviral Matrix, Capsid or Nucleocapsid proteins that help to form the viral core particle. A suitable gag nucleic acid is found at about position 1893 to about position 3575 of SEQ ID NO:122. This region encodes a Gag polypeptide having amino acid sequence SEQ ID NO:140.

A protease nucleic acid can have an open reading frame for a polypeptide with retroviral protease activity capable of processing Gag/Pol polyproteins into retroviral Matrix, Capsid, Nucleocapsid and Polymerase proteins. A suitable nucleic acid sequence for a protease is found at about position 3576 to about position 4556 of SEQ ID NO:122). This sequence encodes a protease polypeptide with the amino acid sequence set forth in SEQ ID NO:141.

A reverse transcriptase nucleic acid can provide an open reading frame for a polypeptide with reverse transcriptase activity. Suitable nucleic acid sequences for a reverse transcriptase include, for example, those found at about position 4602 to about position 6314 of SEQ ID NO:122 and those set forth in SEQ ID NO:138. The nucleotide sequence of SEQ ID NO:138 encodes a polypeptide that has amino acid sequence SEQ ID NO:139 and that can synthesize cDNA from RNA.

An integrase nucleic acid can provide an open reading frame for a polypeptide that can facilitate integration of a nucleic acid containing one or two partial or complete LTR(s) that have recessed 3′OH ends. A suitable nucleic acid sequence for an integrase is found at about position 6315 to about position 7625 of SEQ ID NO:122. This nucleic acid sequence encodes a polypeptide that has the amino acid sequence set forth in SEQ ID NO:142.

An envelope nucleic acid can provide an open reading frame for a polypeptide that makes a retroviral particle infective. A suitable nucleic acid sequence for an envelope polypeptide is found at about position 8745 to about position 10600 of SEQ ID NO:122. This nucleic acid sequence encodes a polypeptide that has amino acid sequence SEQ ID NO:129.

In another embodiment, the envelope nucleic acid resides at about position 8745 to about position 10673 of SEQ ID NO:122. This envelope nucleic acid sequence can be translated by read through of a predicted stop codon to generate an envelope polypeptide having SEQ ID NO:130. In another embodiment, the envelope nucleic acid resides at about position 8745 to about position 10728 of SEQ ID NO:122. This envelope sequence can be translated through a frame shift to generate an envelope polypeptide having SEQ ID NO:131.

A PPT (e.g., PPT1 or PPT2) nucleic acid can facilitate second strand synthesis, for example, by providing a primer site for second strand (plus) synthesis of a retroviral genome. Typically, a PPT such as PPT2 can be used to facilitate second strand synthesis. A suitable PPT2 resides at about position 10738 to about position 10747 of SEQ ID NO:122. A PPT such as PPT1 may be needed to form a triplex flap necessary for nuclear import of the cDNA. A suitable PPT™ resides at about position 12205 to about position 12218 of SEQ ID NO:122.

A non-coding region can be found at about position 10729 to about position 12219 of SEQ ID NO:122. This non-coding region can provide cis-acting sequences for replication and in some cases for formation of the triplex flap that generally is needed for nuclear importation of the retroviral cDNA. A non-coding region such as that found at about position 7626 to about position 8744 of SEQ ID NO:122 can provide cis-acting sequences for replication and in some cases for the expression of envelope polypeptides.

A splice site acceptor site can facilitate splicing of an RNA (e.g., a viral RNA) to form a mature RNA that can be properly translated into a polypeptide (e.g., an envelope polypeptide). A suitable splice site acceptor site can be found at about position 8736 to about position 8739 of SEQ ID NO:122.

A 3′ LTR can provide promoter, polyadenylation, transcript termination, enhancer, and/or silencer function. A 3′ LTR also can provide end sequences that are recognized by integrase and used for insertion of retroviral cDNA (or heterologous DNA) into the genome of a host cell. A suitable nucleic acid sequence for a 3′ LTR can be found at about position 12220 to about position 13966 of SEQ ID NO:122.

A transcript termination site can be found at about position 12963 to about position 12993 of SEQ ID NO:122.

The nucleic acids and vectors described herein need not have the exact nucleic acid sequences described herein. Instead, the sequences of these nucleic acids and vectors can vary, and often either perform a desired function or have some other utility, for example, as a nucleic acid probe for complementary nucleic acids. For example, some sequence variability can be present in a 5′ LTR, promoter, primer binding site, gag, protease, reverse transcriptase, integrase, envelope, polypurine tract, 3′ LTR, and transcript termination site nucleic acid, and yet these elements can retain their specified functions.

Fragments and variant nucleic acids also are encompassed by the invention. Nucleic acid “fragments” can be of two general types. First, fragment nucleic acids can be less than full-length and still perform their intended function. Second, fragments of nucleic acids identified herein can be useful as hybridization probes even though they may have lower than normal levels of activity or function. Fragments of a nucleic acid of the invention can be at least about 10 nucleotides in length (e.g., about 15 nucleotides, about 17 nucleotides, about 18 nucleotides, about 20 nucleotides, about 50 nucleotides, about 100 nucleotides or more than 100 nucleotides in length). In general, a fragment nucleic acid of the invention can have any upper size limit so long as it is related in sequence to the nucleic acids of the invention but is not full length.

As indicated above, “variants” are substantially similar or substantially homologous sequences. For nucleotide sequences that encode proteins, “variants” include those sequences that, because of the degeneracy of the genetic code, encode the identical amino acid sequence of the reference protein. Variant nucleic acids also include those that encode polypeptides that do not have amino acid sequences identical to that of the proteins identified herein, but that encode an active protein with conservative changes in the amino acid sequence.

As is known by one of skill in the art, the genetic code is “degenerate,” meaning that several trinucleotide codons can encode the same amino acid. This degeneracy is apparent from Table 1.

TABLE 1

| | Second Position | | | | |
|---|---|---|---|---|---|
| 1^{st} Position | T | C | A | G | 3^{rd} Position |
| T | TTT = Phe | TCT = Ser | TAT = Tyr | TGT = Cys | T |
| T | TTC = Phe | TCC = Ser | TAC = Tyr | TGC = Cys | C |
| T | TTA = Leu | TCA = Ser | TAA = Stop | TGA = Stop | A |
| T | TTG = Leu | TCG = Ser | TAG = Stop | TGG = Trp | G |
| C | CTT = Leu | CCT = Pro | CAT = His | CGT = Arg | T |
| C | CTC = Leu | CCC = Pro | CAC = His | CGC = Arg | C |
| C | CTA = Leu | CCA = Pro | CAA = Gln | CGA = Arg | A |
| C | CTG = Leu | CCG = Pro | CAG = Gln | CGG = Arg | G |
| A | ATT = Ile | ACT = Thr | AAT = Asn | AGT = Ser | T |
| A | ATC = Ile | ACC = Thr | AAC = Asn | AGC = Ser | C |
| A | ATA = Ile | ACA = Thr | AAA = Lys | AGA = Arg | A |
| A | ATG = Met | ACG = Thr | AAG = Lys | AGG = Arg | G |
| G | GTT = Val | GCT = Ala | GAT = Asp | GGT = GIy | T |
| G | GTC = Val | GCC = Ala | GAC = Asp | GGC = Gly | C |
| G | GTA = Val | GCA = Ala | GAA = Gln | GGA = Gly | A |
| G | GTG = Val | GCG = Ala | GAG = Gln | GGG = Gly | G |

Hence, many changes in the nucleotide sequence of the variant may be silent and may not alter the amino acid sequence encoded by the nucleic acid. Where nucleic acid sequence alterations are silent, a variant nucleic acid will encode a polypeptide with the same amino acid sequence as the reference nucleic acid. Therefore, a particular nucleic acid sequence of the invention also encompasses variants with degenerate codon substitutions, and complementary sequences thereof, as well as the sequence explicitly specified by a SEQ ID NO. Specifically, degenerate codon substitutions may be achieved by generating sequences in which the reference codon is replaced by any of the codons for the amino acid specified by the reference codon. In general, the third position of one or more selected codons can be substituted with mixed-base and/or deoxyinosine residues as disclosed by Batzer et al. (1991) Nucleic Acid Res. 19: 5081 and/or Ohtsuka et al. (1985) J. Biol. Chem. 260: 2605; Rossolini et al. (1994) Mol. Cell. Probes 8: 91.

In some embodiments, a nucleic acid of the invention encodes a polypeptide. For example, a nucleic acid can encode a Gag polypeptide, a protease polypeptide, a reverse transcriptase polypeptide, an integrase polypeptide, or an envelope polypeptide. An example of a nucleic acid that can encode a reverse transcriptase polypeptide having amino acid sequence SEQ ID NO:139 is a nucleic acid having the nucleotide sequence shown in SEQ ID NO:138. However, as indicated by Table 1, other nucleic acids also can encode a polypeptide containing the amino acid sequence of SEQ ID NO:139, and the invention is directed to all such nucleic acids. The same is true for the other Gag, Gag/Pol, PR, RT, IN, and Env polypeptides provided herein. Accordingly, the invention is directed to all nucleic acids that can encode any of the polypeptides provided herein.

Moreover, the invention is not limited to silent changes in the present nucleotide sequences but also includes variant nucleic acid sequences that conservatively alter the amino acid sequence of a polypeptide of the invention. According to the present invention, variant and reference nucleic acids of the invention may differ in the encoded amino acid sequence by one or more substitutions, additions, insertions, deletions, fusions, and truncations, which may be present in any combination so long as an active protein is encoded by the variant nucleic acid. Such variant nucleic acids will not encode exactly the same amino acid sequence as the reference nucleic acid, but typically will have conservative sequence changes.

In some embodiments, variant nucleic acids with silent and conservative changes can be defined and characterized by the degree of sequence identity to a reference nucleic acid. As recognized by one of skill in the art, such nucleic acids can hybridize under stringent conditions with the reference nucleic acid. Accordingly, a nucleic acid of the invention has at least 80 percent sequence identity (e.g., at least 85 percent, at least 90 percent, at least 92 percent, at least 95 percent, at least 97 percent, at least 98 percent, or at least 99 percent identity) to the nucleotide sequence set forth in SEQ ID NO:122, a fragment of SEQ ID NO:122, or the complementary strand of SEQ ID NO:122 or fragment of SEQ ID NO:122. Isolated nucleic acid molecules of the invention thus contain a nucleic acid sequence having (1) a length, and (2) a percent identity to an identified nucleic acid sequence over that length. The invention also provides isolated nucleic acid molecules that contain a nucleic acid sequence encoding a polypeptide that contains an amino acid sequence having (1) a length, and (2) a percent identity to an identified amino acid sequence over that length. Typically, the identified nucleic acid or amino acid sequence is a sequence referenced by a particular sequence identification number, and the nucleic acid or amino acid sequence being compared to the identified sequence is referred to as the target sequence. For example, an identified nucleotide sequence can be the sequence set forth in SEQ ID NO:122 or a fragment of SEQ ID NO:122, and an identified amino acid sequence can be the sequence set forth in SEQ ID NO:128, 129, 130, or 131.

A length and percent identity over that length for any nucleic acid or amino acid sequence is determined as follows. First, a nucleic acid or amino acid sequence is compared to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the Bl2seq program can be found in the readme file accompanying BLASTZ.

Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q −1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides or amino acid residues from the target sequence presented in alignment with sequence from the identified sequence starting with any matched position and ending with any other matched position. A matched position is any position where an identical nucleotide or amino acid residue is presented in both the target and identified sequence. Gaps presented in the target sequence are not counted since gaps are not nucleotides or amino acid residues. Likewise, gaps presented in the identified sequence are not counted since target sequence nucleotides or amino acid residues are counted, not nucleotides or amino acid residues from the identified sequence.

The percent identity over a determined length is determined by counting the number of matched positions over that length and dividing that number by the length followed by multiplying the resulting value by 100. For example, if (1) a 10,000 nucleotide target sequence is compared to the sequence set forth in SEQ ID NO:122, (2) the Bl2seq program presents 9000 nucleotides from the target sequence aligned with a region of the sequence set forth in SEQ ID NO:122 where the first and last nucleotides of that 9000 nucleotide region are matches, and (3) the number of matches over those 9000 aligned nucleotides is 8500, then the 10,000 nucleotide target sequence contains a length of 9000 and a percent identity over that length of 94 (i.e., 8500/9000*100=94).

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

Variant nucleic acids can be detected and isolated by standard hybridization procedures. Hybridization to detect or isolate such sequences is generally carried out under stringent conditions. “Stringent hybridization conditions” and “stringent wash conditions” in the context of nucleic acid hybridization experiments such as Southern and Northern hybridization are sequence dependent, and are different under different environmental parameters. Longer sequences hybridize specifically at higher temperatures. An extensive guide to the hybridization of nucleic acids is found in Tijssen, Laboratory Techniques in Biochemistry and Molecular biology—Hybridization with Nucleic Acid Probes, page 1, chapter 2 “Overview of principles of hybridization and the strategy of nucleic acid probe assays” Elsevier, New York (1993). See also, Sambrook et al., Molecular Cloning: A Laboratory Manual, 2^{nd} ed., Cold Spring Harbor Press, N.Y., pp 9.31-9.58 (1989); and Sambrook et al., Molecular Cloning: A Laboratory Manual, 3^{rd} ed., Cold Spring Harbor Press, N.Y. (2001).

The invention also provides methods for detection and isolation of derivative or variant nucleic acids encoding the proteins provided herein. The methods can involve hybridizing at least a portion of a nucleic acid comprising any one of the nucleotide sequences identified herein to a sample nucleic acid, thereby forming a hybridization complex; and detecting the hybridization complex. The presence of the complex correlates with the presence of a derivative or variant nucleic acid which can be further characterized by nucleic acid sequencing, expression of RNA and/or protein and testing to determine whether the derivative or variant retains activity. In general, the portion of a nucleic acid that is used for hybridization is at least fifteen nucleotides in length, and hybridization is under hybridization conditions that are sufficiently stringent to permit detection and isolation of substantially homologous nucleic acids. In an alternative embodiment, a nucleic acid sample is amplified by the polymerase chain reaction (PCR) using primer oligonucleotides selected from any one of the nucleotide sequences identified herein.

Generally, highly stringent hybridization and wash conditions are selected to be about 5° C. lower than the thermal melting point (Tₘ) for the specific double-stranded sequence at a defined ionic strength and pH. For example, under “highly stringent conditions” or “highly stringent hybridization conditions” a nucleic acid will hybridize to its complement to a detectably greater degree than to other sequences (e.g., at least 2-fold over background). By controlling the stringency of the hybridization and/or the washing conditions, nucleic acids having 100% complementary can be identified and isolated.

Alternatively, stringency conditions can be adjusted to allow some mismatching in sequences so that lower degrees of similarity are detected (heterologous probing). Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl and 0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

The degree of complementarity or homology of hybrids obtained during hybridization is typically a function of post-hybridization washes, the critical factors being the ionic strength and temperature of the final wash solution. The type and length of hybridizing nucleic acids also affects whether hybridization will occur and whether any hybrids formed will be stable under a given set of hybridization and wash conditions. For DNA-DNA hybrids, the Tₘ can be approximated from the equation of Meinkoth and Wahl (1984) Anal. Biochem. 138:267-284 (1984);

Tₘ=81.5° C.+16.6(logM)+0.41(% GC)−0.61(% form)−500/L

where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Very stringent conditions are selected for hybridization to derivative and variant nucleic acids having a Tₘ equal to the exact complement of a particular probe, less stringent conditions are selected for hybridization to derivative and variant nucleic acids having a Tₘ less than the exact complement of the probe.

In general, Tₘ is reduced by about 1° C. for each 1% of mismatching. Thus, Tₘ, hybridization, and/or wash conditions can be adjusted to hybridize to sequences of the desired sequence identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ).

An example of stringent hybridization conditions for hybridization of complementary nucleic acids which have more than 100 complementary residues on a filter in a Southern or Northern blot is 50% formamide with 1 mg of heparin at 42° C., with the hybridization being carried out overnight. An example of highly stringent conditions is 0.1 5 M NaCl at 72° C. for about 15 minutes. An example of stringent wash conditions is a 0.2×SSC wash at 65° C. for 15 minutes (see also, Sambrook, supra). Often, a high stringency wash is preceded by a low stringency wash to remove background probe signal. An example of medium stringency for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C.

Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to detect and isolate homologous nucleic acids that are substantially identical to reference nucleic acids of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1 ×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (supra); Ausubel et al., eds. (1995) Current Protocols in Molecular Biology, Chapter 2 (Greene Publishing and Wiley—Interscience, New York); and Sambrook et al., 1989 (supra). Using these references and the teachings herein on the relationship between Tₘ, mismatch, and hybridization and wash conditions, those of ordinary skill can generate variants of the present nucleic acids.

Nucleic acids of the present invention can identify polymorphic loci that can serve as molecular markers. Molecular markers are useful in plant breeding to determine the relatedness of two plant lines or to monitor quantitative trait loci (QTL) in a plant breeding program. The term “quantitative trait loci” has been used to describe variability in expression of a phenotypic trait that shows continuous variability and is the net result of multiple genetic loci. It is estimated that 98% of the economically important phenotypic traits in domesticated plants are quantitative traits. These traits are classified as oligogenic or polygenic based on the perceived numbers and magnitudes of segregating genetic factors affecting variability in expression of the phenotypic trait. Phenotypic traits associated with QTL are quantitative, meaning that, in some context, a numerical value can be ascribed to the trait. Phenotypic traits associated with QTL include, but are not limited to, grain yield, grain moisture, grain oil, root lodging, stalk lodging, plant height, ear height, disease resistance, and insect resistance.

Molecular markers can, therefore, be used as a measure of genotype at a linked locus (e.g., a QTL) that may otherwise be difficult to score. Molecular markers include restriction fragment length polymorphisms (RFLPs), simple sequence repeats (SSRs), arbitrary fragment length polymorphisms (AFLPs), and randomly amplified polymorphic DNA (RAPDs). See, e.g., U.S. Pat. Nos. 5,746,023 and 5,126,239. Nucleic acids of the present invention can identify additional polymorphic loci that can serve as molecular markers. Nucleic acids of the invention that are useful for identifying polymorphic loci can be, for example, of a length suitable for PCR primers (e.g., about 16 to about 25 nucleotides in length), or can be of a length suitable for a restriction fragment length polymorphism (RFLP) probe (e.g., about 100 to about 1500 nucleotides in length).

Polypeptides

The invention provides novel polypeptides and fragments thereof. In some embodiments, such polypeptides are enzymatically active. For example, the invention provides Gag polypeptides, protease polypeptides, reverse transcriptase polypeptides, integrase polypeptides, and envelope polypeptides. Polypeptides of the invention typically are substantially purified polypeptides. In particular, isolated polypeptides of the invention typically are substantially free of proteins normally present in A. thaliana and Agrobacterium tumefaciens.

Polypeptides provided herein have at least 85 percent amino acid sequence identity (e.g., at least 85 percent, at least 90 percent, at least 95 percent, or at least 98 percent identity) to amino acid sequences encoded by an open reading frame found in SEQ ID NO:122 (e.g., the amino acid sequences of SEQ ID NOS:128, 129, 130, 131, 139, 140, 141, and 142). The percent identity of a particular amino acid sequence to SEQ ID NO:122 is determined as disclosed above. Typically, the polypeptides provided herein are at least 50 amino acids in length (e.g., 50, 75, 100, or more than 100 amino acids in length).

In one embodiment, the invention provides a Gag polypeptide (e.g., a Gag polypeptide that is encoded by nucleotides 1893 to 3575 of SEQ ID NO:122 and thus has the amino acid sequence set forth in SEQ ID NO:140, or a Gag polypeptide having an amino acid sequence that is at least 85 percent identical to the amino acid sequence encoded by nucleotides 1893 to 3575 of SEQ ID NO:122). Significant portions of the Gag polypeptide sequence encoded by SEQ ID NO:122 are distinct from other Gag polypeptide sequences. For example, a region encompassing amino acid positions 130-135 (LFPFSL, SEQ ID NO:143) and a region spanning amino acid positions 191-196 (EAWERF, SEQ ID NO:144) are distinct from other Gag polypeptide sequences. Accordingly, the invention is also directed to a Gag polypeptide containing amino acid SEQ ID NOS:143 and 144.

In another embodiment, the invention provides a PR polypeptide (e.g., a PR polypeptide that is encoded by nucleotides 3576 to 4556 of SEQ ID NO:122 and thus has the amino acid sequence set forth in SEQ ID NO:141, or a PR polypeptide having an amino acid sequence that is at least 85 percent identical to the amino acid sequence encoded by nucleotides 3576 to 4556 of SEQ ID NO:122). Significant portions of the protease sequence encoded by SEQ ID NO:122 are distinct from other protease sequences. For example, a region encompassing amino acid positions 694-699 (DLGASV, SEQ ID NO:145) is distinct from other protease sequences. Accordingly, the invention is also directed to a PR polypeptide having amino acid SEQ ID NO:145. PR polypeptides of the invention can be useful for catalyzing the cleavage of particular polyproteins into individual proteins or into protein fragments. The ability of a polypeptide to function as a PR can be assessed as described in Example 5, for example.

In another embodiment, the invention provides a RT polypeptide (e.g., a RT polypeptide that is encoded by nucleotides 4602 to 6314 of SEQ ID NO:122, a RT polypeptide that is encoded by the nucleotide sequence set forth in SEQ ID NO:138 and thus has the amino acid sequence set forth in SEQ ID NO:139, or a RT polypeptide having an amino acid sequence that is at least 85 percent identical to the amino acid sequence encoded by nucleotides 4602 to 6314 of SEQ ID NO:122). Significant portions of the RT polypeptide sequence encoded by SEQ ID NO:122 are distinct from other reverse transcriptase polypeptide sequences. For example, a region encompassing amino acid positions 1177-1181 (FMDDF, SEQ ID NO:146) is distinct from other reverse transcriptase polypeptide sequences. Accordingly, the invention is also directed to a RT polypeptide containing amino acid SEQ ID NO:146. RT polypeptides provided herein can be useful to catalyze the synthesis of cDNA from mRNA. For example, RT can catalyze the incorporation of deoxynucleotides into a cDNA molecule, using mRNA as a template and oligo(dT) as a primer. The RT polypeptides provided herein can have a range of activities. Typically, one “unit” of RT can catalyze the incorporation of 1 nmol dNTP into acid- (e.g., trichloroacetic acid-) precipitatable material in 10 minutes. As such, functional RT polypeptides can be used to prepare double-stranded nucleic acid molecules from RNA molecules.

The invention also provides an IN polypeptide (e.g., an IN polypeptide that is encoded by nucleotides 6315 to 7625 of SEQ ID NO:122 and thus has the amino acid sequence set forth in SEQ ID NO:142, or an IN polypeptide having an amino acid sequence that is at least 85 percent identical to the amino acid sequence encoded by nucleotides 6315 to 7625 of SEQ ID NO:122). Significant portions of the IN polypeptide sequence encoded by SEQ ID NO:122 are distinct from other integrase polypeptide sequences. For example, regions encompassing amino acid positions 1738-1749 (KLDDALWAYRTA, SEQ ID NO:147) and amino acid positions 1883-1889 (VNGQRLK, SEQ ID NO:148) are distinct from other integrase polypeptide sequences. Accordingly, the invention is also directed to an integrase polypeptide containing amino acid SEQ ID NO:147 and SEQ ID NO:148.

In another embodiment, the invention provides an Env polypeptide (e.g., an Env polypeptide that is encoded by nucleotides 8745 to 10600, nucleotides 8745 to 10673, or nucleotides 8745 to 10728 of SEQ ID NO:122 and thus has the amino acid sequence set forth in SEQ ID NO:129, SEQ ID NO:130, or SEQ ID NO:131, respectively, or an Env polypeptide having an amino acid sequence that is at least 85 percent identical to the amino acid sequence encoded by nucleotides 8745 to 10600, 8745 to 10673, or 8745 to 10728 of SEQ ID NO:122). Significant portions of the envelope polypeptide sequence encoded by SEQ ID NO:122 are distinct from other envelope polypeptide sequences. For example, regions encompassing amino acid positions 1-9 (MSNYSGSSS; SEQ ID NO:149) and amino acid positions 311-336 (RGALCIGGVVTPILIACGVPLISAGL; SEQ ID NO:150) are distinct from other envelope polypeptide sequences. Accordingly, the invention is also directed to an envelope polypeptide containing the amino acid sequences set forth in SEQ ID NO:149 and SEQ ID NO:150.

As indicated above, the amino acid sequence of a polypeptide of the invention can vary from the amino acid sequences set forth in SEQ ID NOS:128, 129, 130, 131, 139, 140, 141, or 142 by amino acid substitutions, deletions, truncations, and insertions.

Methods for making polypeptides that have amino acid sequences that vary from those of SEQ ID NOS:128, 129, 130, 131, 139, 140, 141, or 142 generally are known in the art. For example, amino acid sequence variants of polypeptides can be prepared by mutations in the corresponding DNA. Methods for mutagenesis and nucleotide sequence alterations are well known in the art. See, for example, Kunkel, Proc. Natl. Acad. Sci. USA 82:488 (1985); Kunkel et al., Meth. Enzymol. 154:367 (1987); U.S. Pat. No. 4,873,192; Walker and Gaastra, eds., Techniques in Molecular Biology, MacMillan Publishing Company, New York (1983) and the references cited therein. Guidance as to appropriate amino acid substitutions that do not affect biological activity of the protein of interest may be found in the model of Dayhoff et al., Atlas of Protein Sequence and Structure, Natl. Biomed. Res. Found., Washington, C.D. (1978), herein incorporated by reference.

Variants of the polypeptides having the amino acid sequences shown in SEQ ID NOS:128, 129, 130, 131, 139, 140, 141, or 142 typically have identity with almost all of the amino acid positions of the Gag, PR, RT, IN, and Env polypeptides encoded by SEQ ID NO:122, and can perform the functions that are described herein for them. In other words, a protease, reverse transcriptase, and integrase retains its enzymatic activity, while the Gag and envelope proteins can adequately provide a structural function that helps maintain the structural integrity of viral particles. However, polypeptides having a difference at one to two amino acid positions from the reference polypeptides of the invention still fall within the scope of the invention.

Amino acid residues of the isolated polypeptides and polypeptide derivatives and variants can be genetically encoded L-amino acids, naturally occurring non-genetically encoded L-amino acids, synthetic L-amino acids or D-enantiomers of any of the above. The amino acid notations used herein for the twenty genetically encoded L-amino acids and common non-encoded amino acids are conventional and are as shown in Table 2.

TABLE 2

| | One-Letter | |
|---|---|---|
| Amino Acid | Symbol | Common Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gin |
| Glutamie acid | F | Glu |
| Glycine | G | Gly |
| Histidine | H | His |
| Isoleucine | I | lie |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-Alanine | | BAla |
| 2,3-Diaminopropionie acid | | Dpr |
| α-Aminoisobutyrie acid | | Aib |
| N-Methylglycine (sarcosine) | | MeGly |
| Ornithine | | Orn |
| Citrulline | | Cit |
| t-Butylalanine | | t-BuA |
| t-Butylglycine | | t-BuG |
| N-methylisoleucine | | MeIle |
| Phenylglycine | | Phg |
| Cyclohexylalanine | | Cha |
| Norleucine | | Nle |
| Naphthylalanine | | Nal |
| Pyridylalanine | | |
| 3-Benzothienyl alanine | | |
| 4-Chlorophenylalanine | | Phe(4-Cl) |
| 2-Fluorophenylalanine | | Phe(2-F) |
| 3-Fluorophenylalanine | | Phe(3-F) |
| 4-Fluorophenylalanine | | Phe(4-F) |
| Penicillamine | | Pen |
| 1,2,3,4-Tetrahydro- | | Tic |
| isoquinoline-3-carboxylic acid | | |
| β-2-thienylalanine | | Thi |
| Methionine sulfoxide | | MSO |
| Homoarginine | | HArg |
| N-acetyl lysine | | AcLys |
| 2,4-Diamino butyric acid | | Dbu |
| p-Aminophenylalanine | | Phe(pNH₂) |
| N-methylvaline | | MeVal |
| Homocysteine | | HCys |
| Homoserine | | HSer |
| ε-Amino hexanoic acid | | Aha |
| δ-Amino valeric acid | | Ava |
| 2,3-Diaminobutyric acid | | Dab |

Polypeptide variants that are encompassed within the scope of the invention can have one or more amino acids substituted with an amino acid of similar chemical and/or physical properties, so long as these variant polypeptides retain their function or remain active. Derivative polypeptides can have one or more amino acids substituted with amino acids having different chemical and/or physical properties, so long as these variant polypeptides retain their function and/or activity.

Amino acids that are substitutable for each other in the present variant polypeptides generally reside within similar classes or subclasses. As known to one of skill in the art, amino acids can be placed into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated i-electron system (aromatic group). The aromatic group may be further substituted with substituent groups such as alkyl, alkenyl, alkynyl, hydroxyl, sulfonyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chlorophenylalanine, 2-fluorophenylalanine, 3-fluorophenylalanine and 4-fluorophenylalanine.

“Apolar Amino Acid” refers to a hydrophobic amino acid having a side chain that is generally uncharged at physiological pH and that is not polar. Examples of genetically encoded apolar amino acids include glycine, proline and methionine. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include asparagine and glutamine. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include cysteine. Examples of non-genetically encoded cysteine-like amino acids include homocysteine and penicillamine.

As will be appreciated by those having skill in the art, the above classification is not absolute. Several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a polypeptide.

Certain commonly encountered amino acids that are not genetically encoded and that can be present, or substituted for an amino acid, in the variant polypeptides of the invention include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); .beta.-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). These amino acids also fall into the categories defined above.

The classifications of the above-described genetically encoded and non-encoded amino acids are summarized in Table 3, below. It is to be understood that Table 3 is for illustrative purposes only and does not purport to be an exhaustive list of amino acid residues that may comprise the variant and derivative polypeptides described herein. Other amino acid residues that are useful for making the variant and derivative polypeptides described herein can be found, e.g., in Fasman (1989) CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 3

| Classification | Genetically Encoded | Genetically Non-Encoded |
|---|---|---|
| Hydrophobic | F, L, I, V | |
| Aromatic | F, Y, W | Phg, Nal, Thi, Tic, Phe(4-Cl), |
| | | Phe(2-F), Phe(3-F), Phe(4-F), |
| | | Pyridyl Ala, Bcnzothienyl Ala |
| Apolar | M, G, P | |
| Aliphatic | A, V, L, I | t-BuA, t-BuG, MeJie, Nle, MeVal, |
| | | Cha, bAla, MeGly, Aib |
| Hydrophilic | S, K | Cit, hCys |
| Acidic | D, E | |
| Basic | H, K, R | Dpr, Om, hArg, Phe(p-N112), |
| | | DBU, A₂ BU |
| Polar | Q, N, S, T, Y | Cit, AcLys, MSO, hSer |
| Cysteine-Like | C | Pen, hCys, β-methyl Cys |

Polypeptides of the invention can have any amino acid substituted by any similarly classified amino acid to create a variant peptide, so long as the peptide variant retains its function or activity.

Thus, the polypeptides of the invention encompass both naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. One skilled in the art can readily evaluate the stability, structural integrity and enzymatic activities of the polypeptides and variant polypeptides of the invention by routine screening assays.

The term “purified” with respect to a polypeptide refers to a polypeptide that has been separated from cellular components by which it is naturally accompanied. Typically, the polypeptide is purified when it is at least 60% (e.g., 70%, 80%, 90%, 95%, or 99%), by weight, free from proteins and naturally-occurring organic molecules with which it is naturally associated. In general, an purified polypeptide will yield a single major band on a non-reducing polyacrylamide gel.

Purified polypeptides of the invention can be obtained, for example, by extraction from a natural source, chemical synthesis, or by recombinant production in a host cell. To recombinantly produce a particular polypeptide, a nucleic acid encoding the polypeptide can be ligated into an expression vector and used to transform a prokaryotic (e.g., bacteria) or eukaryotic (e.g., insect, yeast, or mammal) host cell. Polypeptides also can be purified by known chromatographic methods including, for example, DEAE ion exchange, gel filtration, and hydroxylapatite chromatography. See, for example, Flohe et al. (1970) Biochim. Biophys. Acta 220: 469-476; and Tilgmann et al. (1990) FEBS 264: 95-99. Polypeptides can be “engineered” to contain a tag sequence describe herein that allows the polypeptide to be purified (e.g., captured onto an affinity matrix). Immunoaffinity chromatography also can be used to purify polypeptides.

Kits and compositions containing the present polypeptides are substantially free of cellular material. Such preparations and compositions have less than about 30%, 20%, 10%, 5%, (by dry weight) of contaminating plant or plant viral cellular protein.

Vectors and Host Cells

The invention also provides vectors containing a nucleic acid described above. As used herein, a “vector” is a replicon, such as a plasmid, phage, or cosmid, into which another DNA segment may be inserted so as to bring about the replication of the inserted segment. The vectors of the invention can be expression vectors. An “expression vector” is a vector that includes one or more expression control sequences, and an “expression control sequence” is a DNA sequence that controls and regulates the transcription and/or translation of another DNA sequence.

In the expression vectors of the invention, the nucleic acid is operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide including at either the carboxyl or amino terminus.

The invention also relates to host cells (e.g., plant cells) obtained after transfection by the retroelement nucleic acids or vectors of the invention. These host cells can be transfected with the retroelements or vectors of the invention by contacting the host cell with a retroelement or vector provided herein, for a time and under conditions permitting retroviral infection. In some embodiments, a trans-complementing system can be used to provide the gag, pol and env functions that permit transfection of the vectors of the invention. Such a trans-complementing system can include, for example, a vector encoding and capable of expressing the gag, pol and env genes, or a cocktail of proteins encoded by the gag, pol and/or env genes that is capable of facilitating infection, uptake and integration of a vector containing only one or more of the cis-acting retroviral elements of the invention.

Accordingly, a method according to the invention comprises making a host cell (e.g., a plant cell) having a nucleic acid construct described herein. Techniques for introducing exogenous nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,204,253 and 6,013,863. If a cell or tissue culture is used as the recipient tissue for transformation, plants can be regenerated from transformed cultures by techniques known to those skilled in the art. Transgenic plants can be entered into a breeding program, e.g., to introduce a nucleic acid encoding a polypeptide into other lines, to transfer the nucleic acid to other species or for further selection of other desirable traits. Alternatively, transgenic plants can be propagated vegetatively for those species amenable to such techniques. Progeny includes descendants of a particular plant or plant line. Progeny of an instant plant include seeds formed on F₁, F₂, F₃, and subsequent generation plants, or seeds formed on BC₁, BC₂, BC₃, and subsequent generation plants. Seeds produced by a transgenic plant can be grown and then selfed (or outcrossed and selfed) to obtain seeds homozygous for the nucleic acid encoding a novel polypeptide.

Other suitable methods of transformation include, without limitation, the vacuum infiltration method (Bechtold et al. (1993) C.R. Acad. Sci. Paris 316: 1194-1199), the microprojectile bombardment of immature embryos (U.S. Pat. No. 5,990,390) or Type II embryogenic callus cells as described by W. J. Gordon-Kamm et al. ((1990) Plant Cell 2: 603), M. E. Fromm et al. ((1990) Bio/Technology 8: 833) and D. A. Walters et al. ((1992) Plant Molecular Biology 18: 189), or by electroporation of type I embryogenic calluses described by D'Halluin et al. ((1992) The Plant Cell 4: 1495), or by Krzyzek (U.S. Pat. No. 5,384,253). Transformation of plant cells by vortexing with DNA-coated tungsten whiskers (Coffee et al., U.S. Pat. No. 5,302,523) and transformation by exposure of cells to DNA-containing liposomes can also be used. Other methods include micropipette injection, polyethylene glycol (PEG) mediated transformation of protoplasts, and gene gun or particle bombardment techniques. Host cells containing the vectors of the invention can be selected or isolated using the selectable markers or reporter genes described herein. Host cells are cultured using available tissue culture and conditions optimized to allow growth and accumulation of host cells containing the vectors of the invention.

Plants

Plants for use with the vectors of the invention include dicots and monocots, including but not limited to, corn (Zea mays), Brassica sp. (e.g., B. napus, B. rapa, and B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macaclamia integrifolia), almond (Prunus amygdalus), sugar beets (Beta vulgaris), sugarcane (Saccharum spp.), oats, barley, vegetables, ornamentals, and conifers; duckweed (Lemna, see WO 00/07210, which includes members of the family Lemnaceae.

There are four genera and 34 species of duckweed that may be employed in the invention, as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L. turionifera, L. valdiviana); genus Spirodela (S. intermedia, S. polyrrhiza, S. punctata); genus Woffia (Wa. angusta, Wa. arrhiza, Wa. australina, Wa. borealis, Wa. brasiliensis, Wa. columbiana, Wa. elongata, Wa. globosa, Wa. microscopica, Wa. neglecta) and genus Wofiella (Wl. caudata, Wl. denticulata, Wl. gladiata, Wl. hyalina, Wl. lingulata, Wl. repunda, Wl. rotunda, and Wl. neotropica). Any other genera or species of Lemnaceae, if they exist, are also aspects of the present invention. Lemna gibba, Lemna minor, and Lemna miniscula are particularly useful, with Lemna minor and Lemna miniscula being most useful. Lemna species can be classified using the taxonomic scheme described by Landolt, Biosystematic Investigation on the Family of Duckweeds: The family of Lemnaceae—A Monograph Study. Geobatanischen Institut ETH, Stiftung Rubel, Zurich (1986)); vegetables including tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis); and leguminous plants. Plant cells also can be from leguminous plants, such as beans and peas. Beans include guar, locust bean, fenugreek, soybean, garden beans, cowpea, mungbean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Other sources for the polynucleotides of the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, brussel sprouts, onion, carrot, leek, beet, broad bean, celery, radish, pumpkin, endive, gourd, garlic, snapbean, spinach, squash, turnip, asparagus, and zucchini and ornamental plants include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia.

The invention will be further described in the following examples, which do not limit the scope of the invention described in the claims.

### EXAMPLES

### Example 1

### The Athila4 Family of A. thaliana Retroelements

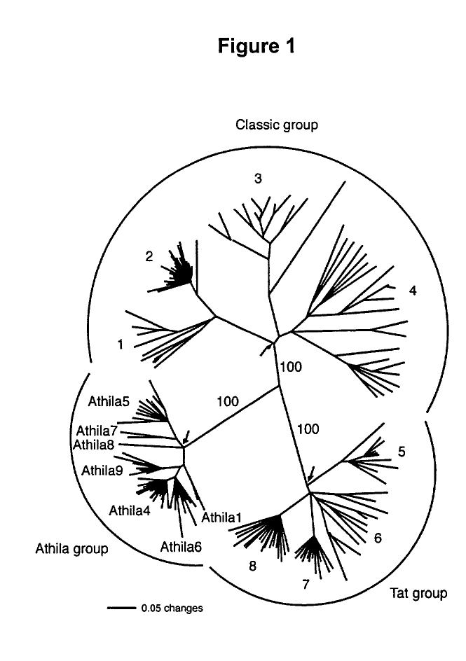

The Athila elements of A. thaliana. To characterize A. thaliana Athila elements, reverse transcriptases from all Ty3-gypsy elements were recovered from the A. thaliana genome sequence (Initiative 2000). BLAST searches (Altschul et al. (1990) J Mol Biol 215: 403-10) were performed with reverse transcriptases from Athila1-1, Tat4-1 and Tma3-1, three divergent A. thaliana Ty3-gypsy elements (Wright and Voytas (1998) supra). Additional BLAST searches were performed with the most divergent retroelement sequences recovered. A total of 191 unique reverse transcriptases were identified. These were aligned, and when necessary, conservative changes were made to correct frameshift mutations. A phylogenetic tree was generated (FIG. 1) by the neighbor-joining method (Saitou and Nei (1987) Mol. Biol. Evol. 4: 406-425) using PAUP v4.0 beta 4a (Swofford (1991 Phylogenetic analysis using parsimony, PAUP. in, Illinois Natural History Survey, Champaign, Ill.). The trees were based on DNA and amino acid sequences that had been aligned with ClustalX v1.63b (Thompson et al. (1994) Nucleic Acids Res. 22: 4673-4680). The A. thaliana Ty3-gypsy elements clustered into three distinct clades designated the classic, Tat and Athila lineages.

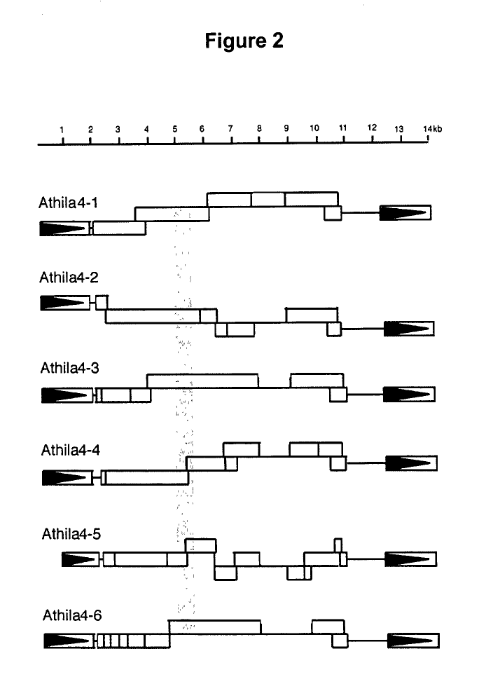

The phylogenetic analysis defined several distinct Athila families (FIG. 1). These included the previously described Athila1 family (Wright and Voytas (1998) supra) and six additional families, designated Athila4-Athila9. The Athila, Athila2 and Athila3 families are not included in the tree, because they have deletions of reverse transcriptase (Pelissier et al. (1995) Plant Mol. Biol. 29: 441-452; Wright and Voytas (1998) supra). Elements in four of the seven families had potential coding regions flanking reverse transcriptase and discernible LTRs (Athila1, Athila4, Athila5, and Athila6). Relatively intact insertions were given species designations (e.g. Athila1-1, FIG. 1). The Athila4 family was the largest and included 22 members. Six of these (designated Athila4-1 to Athila4-6) approximated 14 kb in length and had LTRs of approximately 1.8 kb (FIG. 2). Athila4-3 and Athila4-4 were organized in tandem and shared a central LTR. The tandem Athila4-3/Athila4-4 insertion and the individual Athila4 elements were flanked by 5 bp target site duplications. In pairwise comparisons, the six Athila4 elements averaged 94% nucleotide identity across their entirety. Despite this high degree of sequence identity, gag and pol were broken by stop codons and frameshifts.

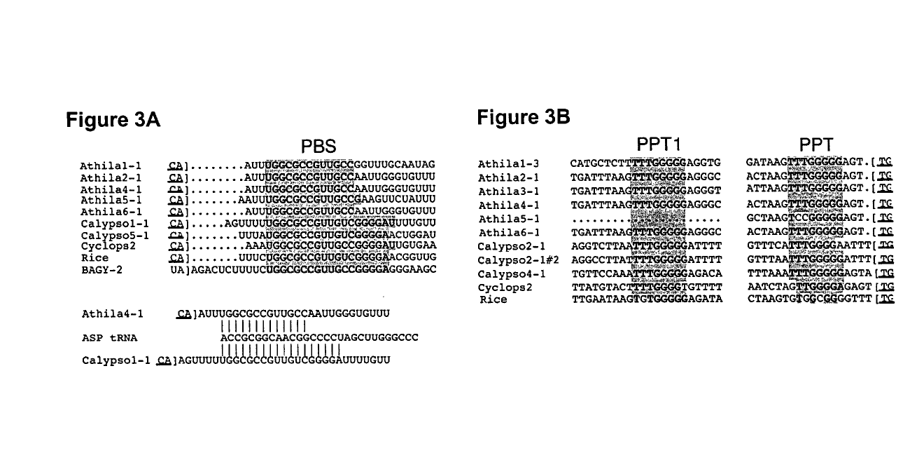

Features of Athila4 elements. For most retroelements, the region adjacent to the 5′ LTR is complementary to a cellular tRNA and serves as the site for priming minus strand DNA synthesis. The PBS of Athila4 and Calypso is complementary to the 3′ end of the aspartic acid tRNA for the GAC codon from A. thaliana and soybean (SEQ ID NO:11; FIG. 3A) (Waldron et al. 1985; Wright and Voytas 1998). Complementarity begins at variable positions from the boundary of the 5′ LTR, and extends for 13 bases for the Athila4 elements. For most retroelements, a stretch of purines adjacent to the 3′ LTR serves as the priming site for plus strand DNA synthesis. A PPT is found at this location in Athila4, and all of the endogenous plant retroelements share a conserved core consensus sequence (TTTGGGGG) as well as less conserved flanking sequences (FIG. 3B). A second PPT motif (PPT1) is found after the env-like gene. The two PPTs delimit a large non-coding region, which in Athila averages ˜2 kb in length (see FIGS. 2 and 3). A second non-coding region lies between gag-pol and the env-like gene and approximates 0.7 kb.

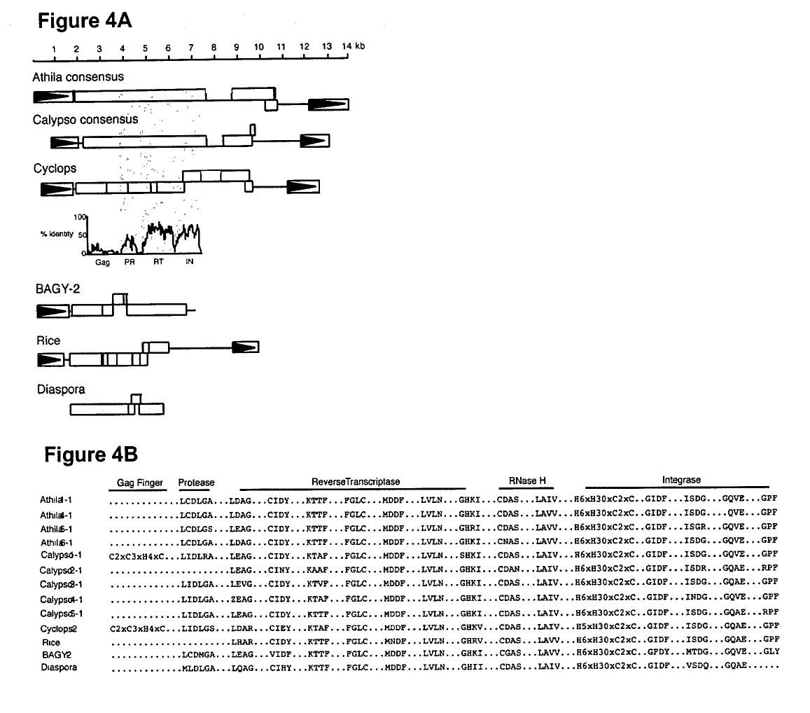

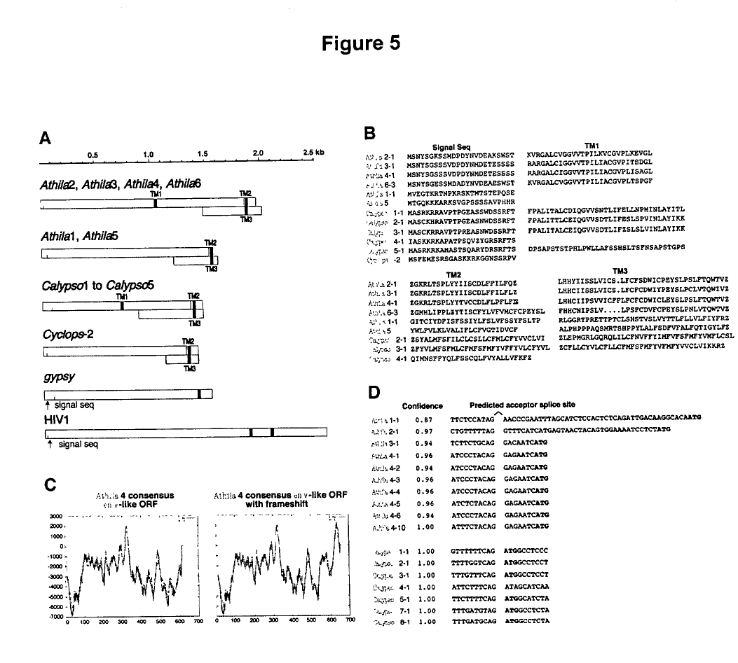

Because of the frameshifts and stop codons in the Athila4 elements, a strict consensus sequence was generated (SEQ ID NO:122). This consensus element was based on sequence alignments between Athila4-1, Athila4-2, Athila4-3, Athila4-4, Athila4-5 and Athila4-6, which were generated using ClustalX (Thompson et al. (1994) supra). FIG. 4A depicts the structural organization of this consensus element as well as Calypso from soybean, Cyclops-2 from pea (Chavanne et al. (1998) supra) and three partially sequenced homologues: Diaspora from soybean, BAGY-2 from barley (Shirasu et al (2000) Genome Res. 10: 908-915) and a degenerate element from rice that was identified from rice genome sequence data. The consensus element encodes Gag and Pol on a single open reading frame of 1911 amino acids. This coding region was aligned with Gag-Pol of Calypso and Cyclops-2, and the percent amino acid identity was plotted along their entirety (FIG. 5A). The first third of the ORF shares about 20% amino acid identity; this region was defined as Gag (˜600 aa) (FIG. 4A). The Calypso and Cyclops-2 Gag proteins encode a conserved finger domain characteristic of retrotransposon and retroviral nucleocapsid proteins (FIG. 4B). This motif is not present in any of the other elements examined. A block of approximately 110 amino acid residues is conserved near the N-terminus of Gag, suggesting a conserved function. Similarity to this region can be detected in the sequence of Diaspora and the rice element but not BAGY-2 (data not shown).

Following Gag is a motif (LI/CDLGA, SEQ ID NO:151) that may be the active site of an aspartic acid protease (FIG. 4B). PR is defined herein as the region of roughly 40% amino acid identity that spans approximately 300 amino acid residues between Gag and RT (shaded region, FIG. 4A). Although the precise boundaries of this PR are not known, this region is considerably larger than the proteases of retrotransposons and retroviruses (e.g. 181 aa for Ty1, 99 aa for HIV (Merkulov et al. (1996) J. Virol 70: 5548-5556; Coffin et al. (1997) supra). Following PR is about 520 amino acids that make up RT. The various RTs share about 68% amino acid identity. All seven conserved amino acid sequence domains characteristic of retroviral and retrotransposon RTs are evident (shaded, FIG. 4A). The remainder of Gag-Pol constitutes an approximately 450 amino acid IN (shaded, FIG. 4A). In addition to the conserved N-terminal zinc binding motif and the DD35E motif of the catalytic domain, IN has a C-terminal extension with a GPY/F module (FIG. 4B) (Malik and Eickbush (1999) supra). The GPY/F module is found in some retroviral and Ty3/gypsy element integrases and is thought to bind DNA. IN shares ˜64% amino acid identity among Athila4, Calypso, and Cyclops-2.

Features of the env-like gene. After gag and pol and between the two non-coding regions, the consensus element encodes an ORF of 619 amino acids (FIG. 5A). Recognizable env-like ORFs are also found in members of the Athila, Athila1-Athila6 and Athila9 families (data not shown). The env-like ORFs of Athila2, Athila3, Athila4 and Athila6 share an average of 69% amino acid sequence identity in pairwise comparisons (data not shown). The Athila1 and Athila5 elements are divergent, and their env-like ORFs do not align well with the other Athila families. No significant amino acid sequence similarity was observed between the pea/soybean and A. thaliana elements.

Retroviral Env proteins typically are transported through the endomembrane system, where they are proteolytically cleaved to generate surface (SU) and transmembrane (TM) proteins prior to being released on the cell surface (Coffin et al. (1997) supra). Targeting to the endomembrane system is mediated by a signal sequence at the N-terminus of env. The N-termini of Athila4 is serine-rich, and the program PSORT (Nakai and Kanehisa (1992) Genomics 14: 897-911) suggests it is targeted to the endoplasmic reticulum (85% confidence).

At the cell surface, the retroviral TM protein spans the plasma membrane. A transmembrane domain was previously reported in the env-like ORFs of several Athila elements (Athila, Athila1, Athila2, Athila3) (Wright and Voytas (1998) supra). The consensus env-like ORF also encodes a transmembrane domain (TM1, FIGS. 5A-5C), to which the program TMpred assigns a score of 2006 (scores above 500 are considered significant) (Hofmann and Stoffel (1993) Biol. Chem. Hoppe-Seyler 347: 166). Similarly, a transmembrane domain is predicted near the center of the Calypso env-like ORF (TMpred value of 947; FIGS. 5A and 5B). The Cyclops-2 env-like protein has a potential transmembrane domain at a similar location, but at a reduced confidence level relative to the other elements (TMpred value of 650).

Analysis of the Athila4 env-like gene indicated a potential to encode additional transmembrane domains after the stop codon. Strong transmembrane domains were predicted in either the same frame as the env-like ORF (TM2, FIGS. 5A-5C) or in the +1 frame (TM3). These potential coding regions extend the env-like ORF to the first PPT (PPT1) and are conserved among some element families (FIG. 5B). Small ORFs with predicted transmembrane domains are also found at the end of the Calypso and Cyclops-2 env-like ORFs. In the consensus Calypso element, the ORF is in a −1 frame, although the degree of degeneracy among Calypso elements reduces confidence in this reading frame assignment. Unfortunately, sequences between Athila families were too divergent to ascertain whether the short ORFs are evolving as coding sequences based on frequencies of synonymous vs. non-synonymous substitutions.

Retroviral env genes are typically expressed from a spliced, subgenomic mRNA (Coffin et al. (1997) supra). A splice site analysis of the consensus element was performed with NetGene2 (Hebsgaard et al., 1996; Brunak et al., 1991). A number of possible splice acceptors were present near the beginning of the env-like gene, one of which is located just before the first methionine and is consistently predicted with a high level of confidence (>94%; FIG. 5D). In the animal retroviruses, the splice site donor is typically located near the 5′ LTR or within Gag. Of the several possible donors in these regions, none are well conserved between element families (data not shown).

To delineate the ends of new Athila elements, the PBS and PPT were identified by a search for the sequences TGGCGCC and TTTGGGGG, respectively. A sequence similar to CAATT adjacent to the PBS is a further clue that identifies a PBS, where the CA is the conserved 3′ dinucleotide end of an LTR. A sequence similar to AGTTG usually is next to the polypurine tract, where the TG is the conserved 5′ dinucleotide end of an LTR.

The PBSs of the retrovirus-like elements that have been described to date, including the Athila4 group, are conserved for the first eleven bases, which consists of the sequence TGGCGCCGTTG (SEQ ID NO:152). The shared PPT sequence is TTTGGGGG (FIG. 3B).

### Example 2

### RNA Expression of Athila Elements in A. thaliana

This example describes reverse transcription-polymerase chain reaction (RT-PCR) amplification of Athila4 mRNA from ddml-2 A. thaliana strains, which have lower levels of DNA methylation. The characterized cDNA clones were derived from several different Athila elements, all of which have a common polyadenylation site in the LTR. The presence of RNA suggested that some Athila elements are actively transcribed in A. thaliana when levels of DNA methylation are reduced.

Retroelement LTRs direct transcription initiation and termination. Transcription initiates within the 5′ LTR and terminates within the 3′LTR downstream of the initiation site. This results in a terminally redundant transcript that is translated to produce retroelement proteins and reverse transcribed to generate cDNA. The end sequences of the Athila4 group LTRs are highly conserved, but the central region (base position about 250 to about 750) is somewhat variable. This is the region that typically contains the promoter and signals for transcription termination and polyadenylation. The LTRs do not have an obvious promoter, nor do they have an obvious polyadenylation signal based on computer prediction programs.

The A. thaliana Athila elements typically are located within heterochromatin flanking the centromeres (Pelissier et al. (1996) Genetica 97: 141-151; The Arabidopsis Genome Initiative (2000) Nature 408: 796-815). These regions contain repeated sequences that are methylated and likely transcriptionally quiescent (Jeddeloh et al. (1999) Genes Dev 12: 1714-1725; Consortium (2000) Cell 100: 377-386). Some Athila group elements and retrotransposons are expressed in genetic backgrounds, such as ddm1, which have reduced levels of DNA methylation (Hirochika et al. (2000) Plant Cell 12: 357-369; Steimer et al. (2000) Plant Cell 12: 1165-1178; Lindroth et al. (2001) Science 292: 2077-2080). To test whether the Athila LTRs can direct transcription, Athila4 mRNAs were sought by RT-PCR in ddm1 backgrounds (Vongs et al. (1993) Science 260: 1926-28). RNA was isolated using the PUREscript RNA isolation kit (Gentra Systems Inc.) and annealed to the primer DVO814 or DVO1247, which are polyT oligos with a specific tail (5′-GGACTTCAGGACTGCTTGACAAA GT₃₀; SEQ ID NO:153), or 5′-GGACTTCAGGACTGCTTGACAAAGT₃₀ (SEQ ID NO:154). First strand DNA synthesis was performed at 42° C. for 2 hours using Superscript II reverse transcriptase and the manufacturer's protocol (Gibco BRL). RNase activity was inhibited by the addition of Super RNase IN per the manufacturer's instructions (Ambion). PCR was carried out using the Expand Long Template PCR System (Roche Molecular Biochemicals) with Athila-specific primers along with DVO385 or DVO1248, which are specific to the tail of DVO814 and DVO1247, respectively. The Athila primers were for five different regions of Athila4

| | | |
|---|---|---|
| (DVO981: | 5′-ATGCATTGATAAGTGTGTATTTTGCATGTCTTG, | SEQ ID NO:155; |
| DVO996: | 5′-ACTCGACCTCCTCACTCTAC, | SEQ ID NO:156; |
| DVO1009: | 5′-AGGACTCTAGGTGAAGTAAG, | SEQ ID NO:157; |
| DVO1119: | 5′-AGGACGTACTCAAGCAACCACTCGACCTTG, or | SEQ ID NO:158; |
| DVO1338: | 5′-TTGGGACTTACCTTTAGCATTC, | SEQ ID NO:159). |

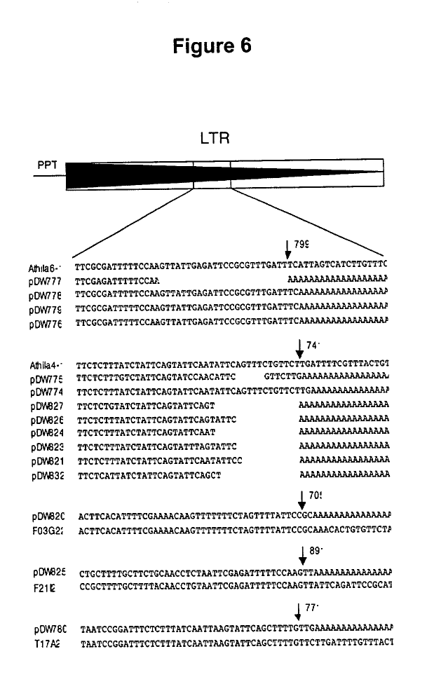

Fifteen separate Athila cDNAs were cloned and sequenced: eight were Athila4 elements, four were Athila6 elements and three could not be easily assigned to a family because of sequence degeneracy (FIG. 6). No transcripts were recovered from a wild type strain. All 15 transcripts terminated within a 200 bp window of a consensus Athila LTR. This suggests that the promoter and polyadenylation signal are located within the first 891 bp of the 5′ LTR and that at least some Athila elements are transcribed in the ddml-2 strain. One of the cDNAs, pDW832, was primed with a gag oligo and the expected 8.4 kb amplification product was obtained. The 1.8 kb of pDW832 that was sequenced matched Athila4-6 except for a single base change, which may have been a result of PCR error. The identification of near full-length Athila cDNAs suggests that transcription initiates in or near the 5′ LTR (Table 4 and FIG. 6).

TABLE 4

Athila cDNA clones obtained by RT-PCR

| | | | |
|---|---|---|---|
| | Length to polyA | | |
| Clone | tail | Primers | Similarity |

| | | | | |
|---|---|---|---|---|
| pDW774 | 792 | bp | DVO981/1248 | Athila4 group |
| pDW775 | 780 | bp | DVO981/385 | Athila4 group |
| pDW776 | 469 | bp | DVO996/1248 | Athila6 group |
| pDW777 | 442 | bp | DVO996/1248 | Athila6 group |
| pDW778 | 440 | bp | DVO996/385 | Athila6 group |
| pDW779 | 440 | bp | DVO996/385 | Athila6 group |
| pDW780 | 776 | bp | DVO9811385 | Athila T17A2 |
| pDW820 | About 1500 | bp | DVO1009/1248 | Athila F03G22 |
| pDW821 | About 1500 | bp | DVO1009/1248 | Athila4 group |
| pDW823 | About 1500 | bp | DVO1009/1248 | Athila4 group |
| pDW824 | About 1200 | bp | DVO1338/1248 | Athila4 group |
| pDW825 | About 1200 | bp | DVO1338/1248 | AthilaF21I2 |
| pDW826 | About 1200 | bp | DVO1338/1248 | Athila4 group |
| pDW827 | About 1200 | bp | DVO1338/1248 | Athila4 group |
| pDW832 | About 8400 | bp | DVO1119/1248 | Athila4-6 |

### Example 3

### A Consensus Retroelement

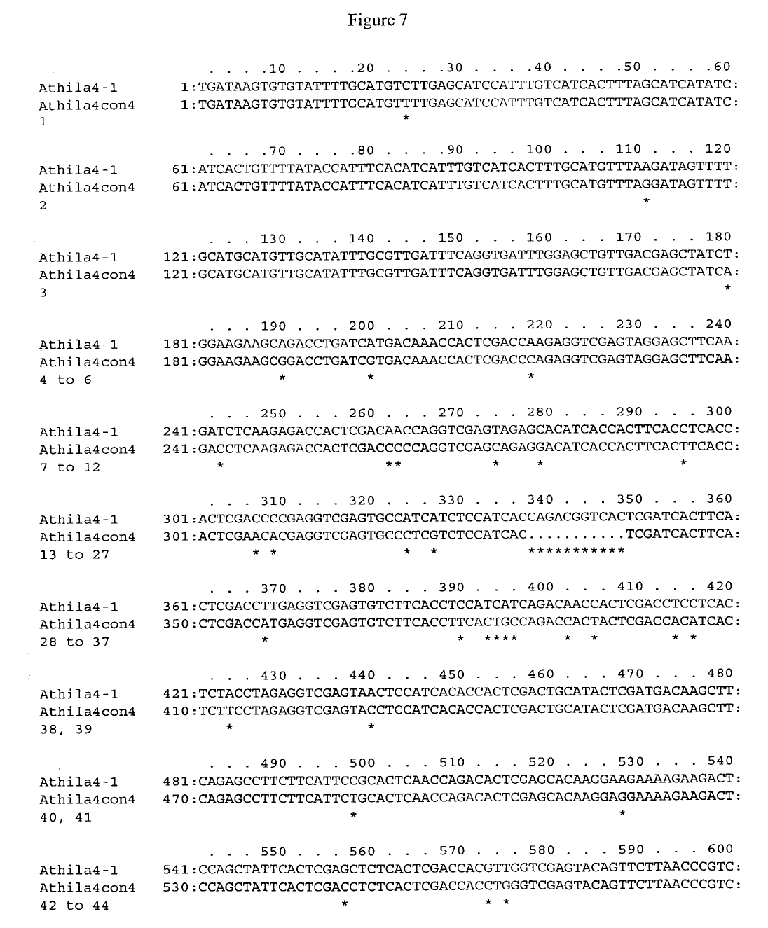

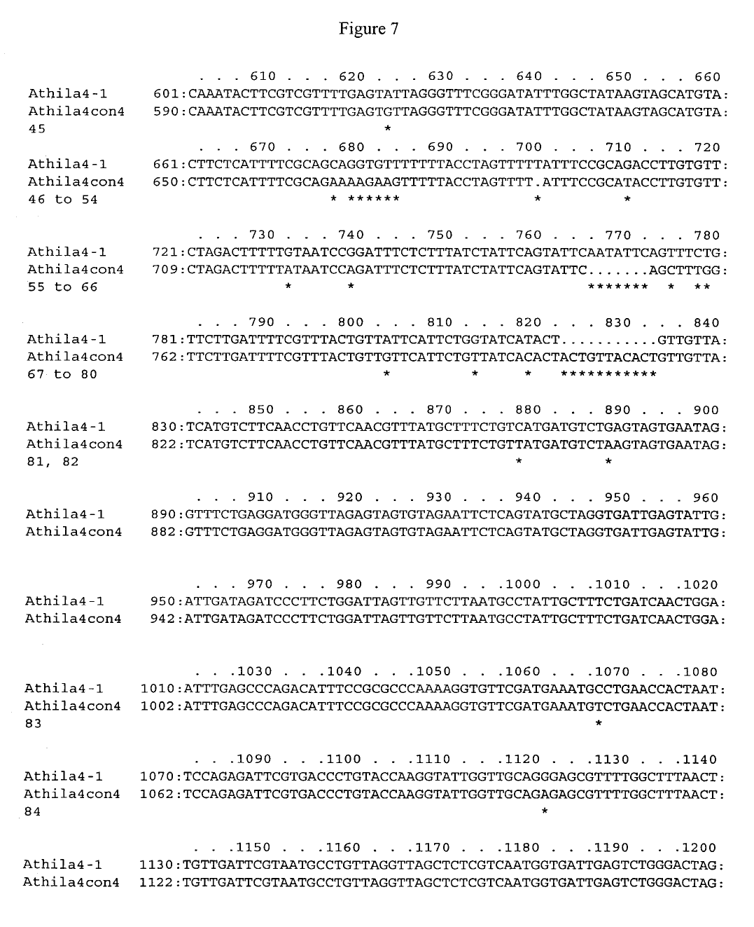

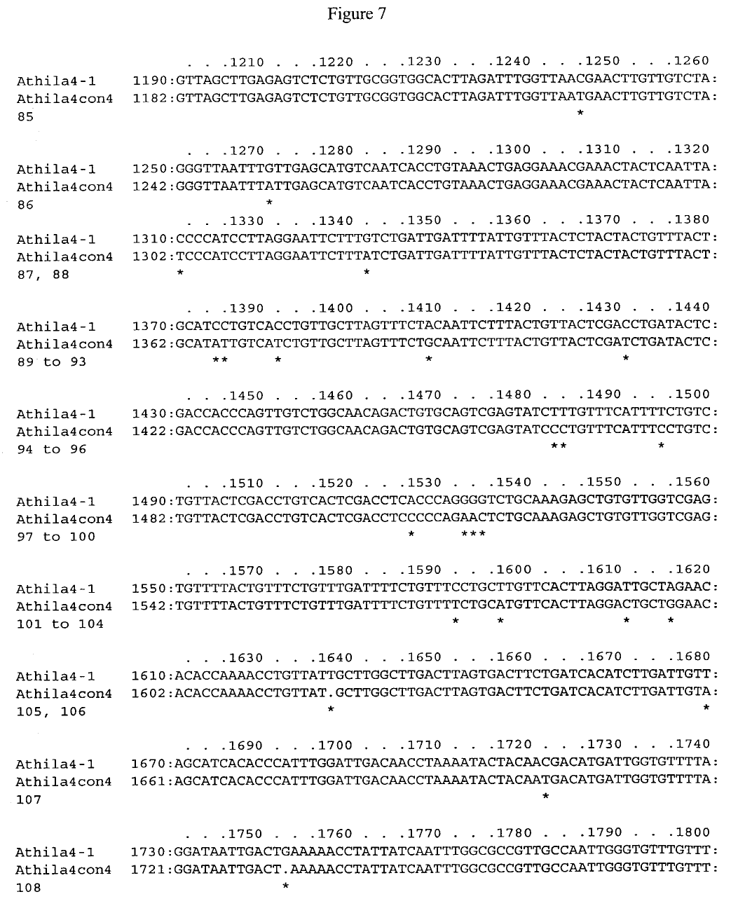

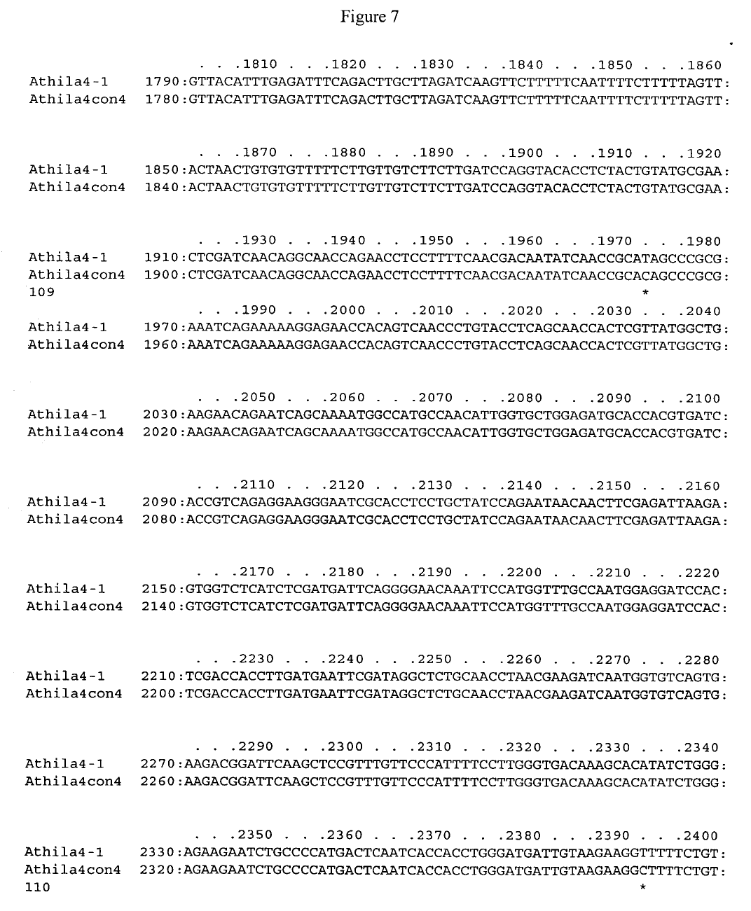

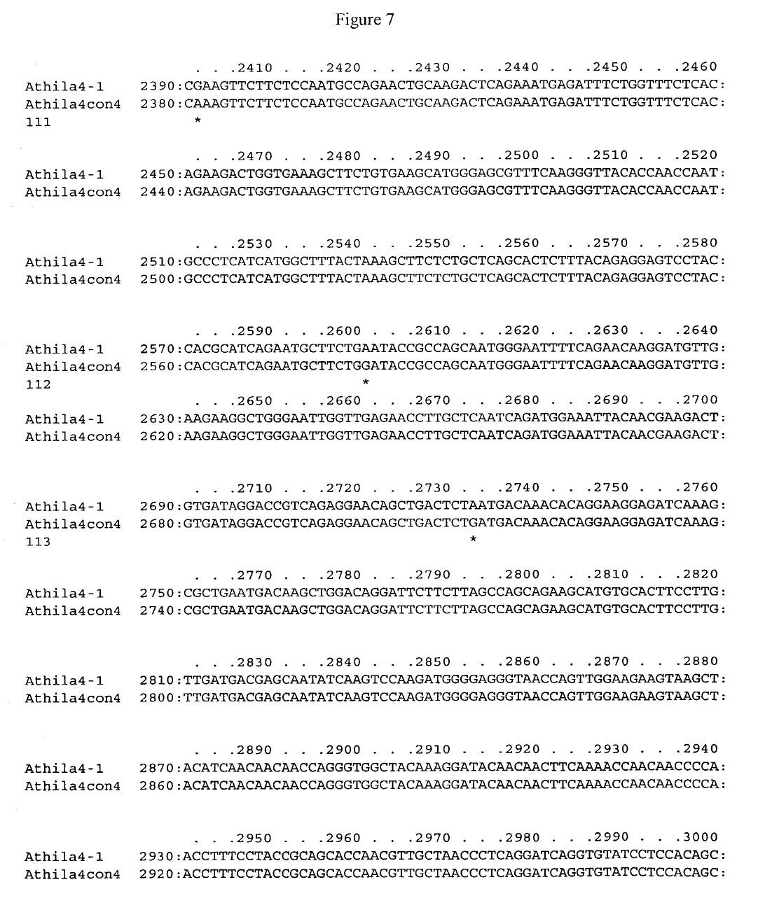

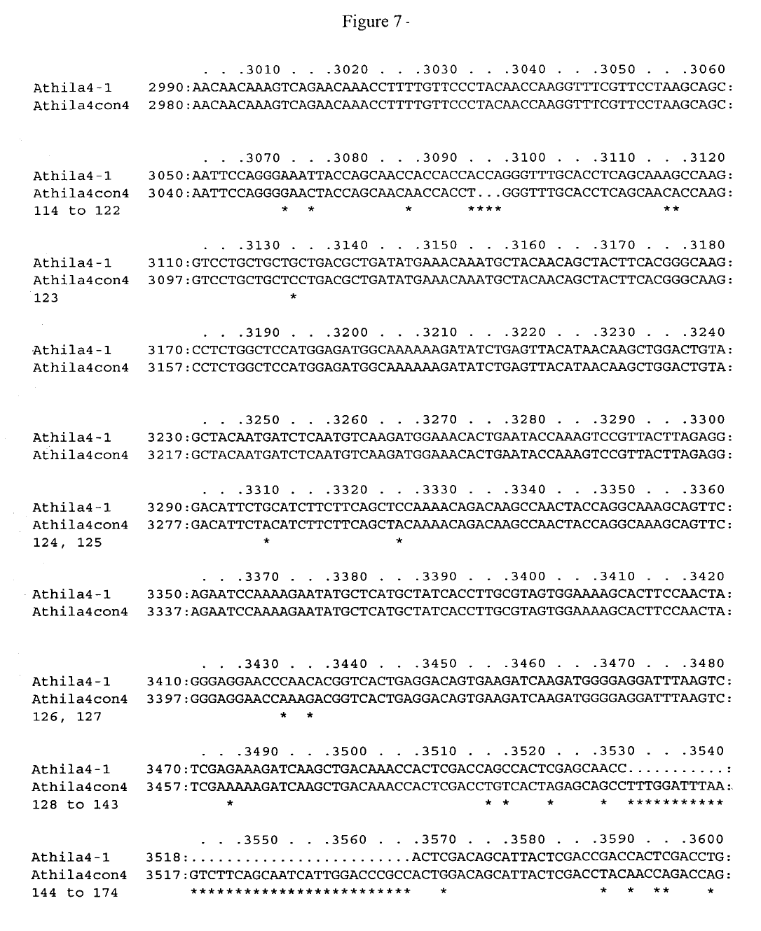

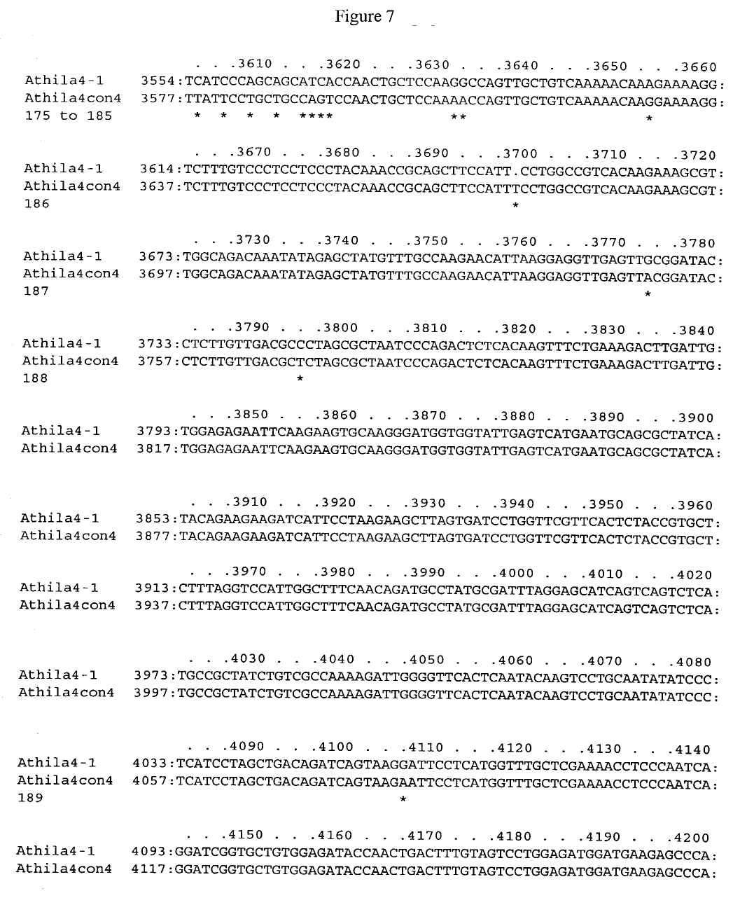

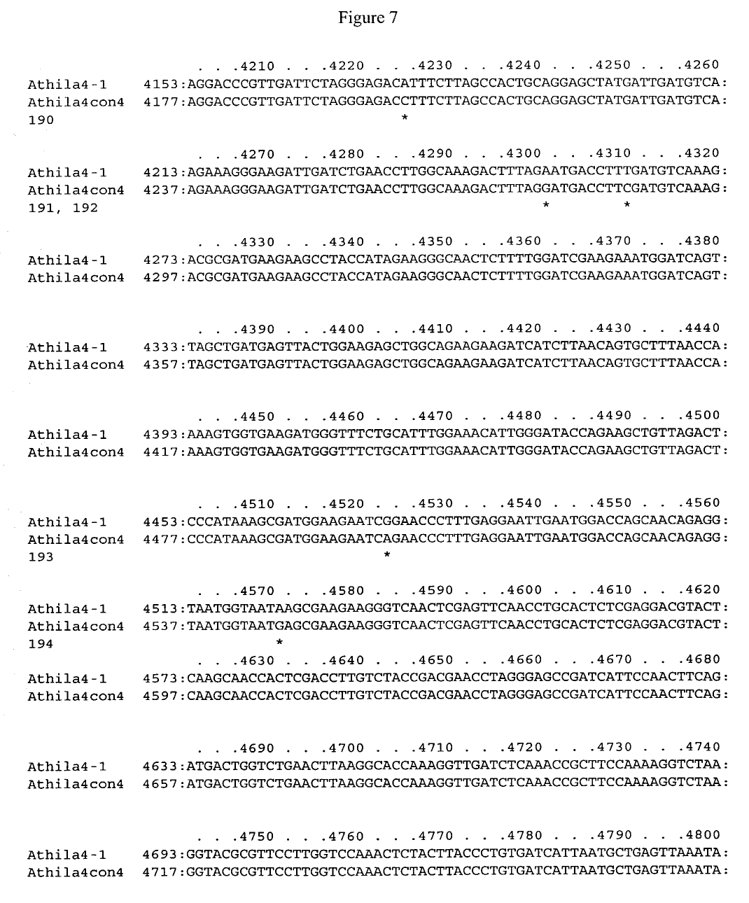

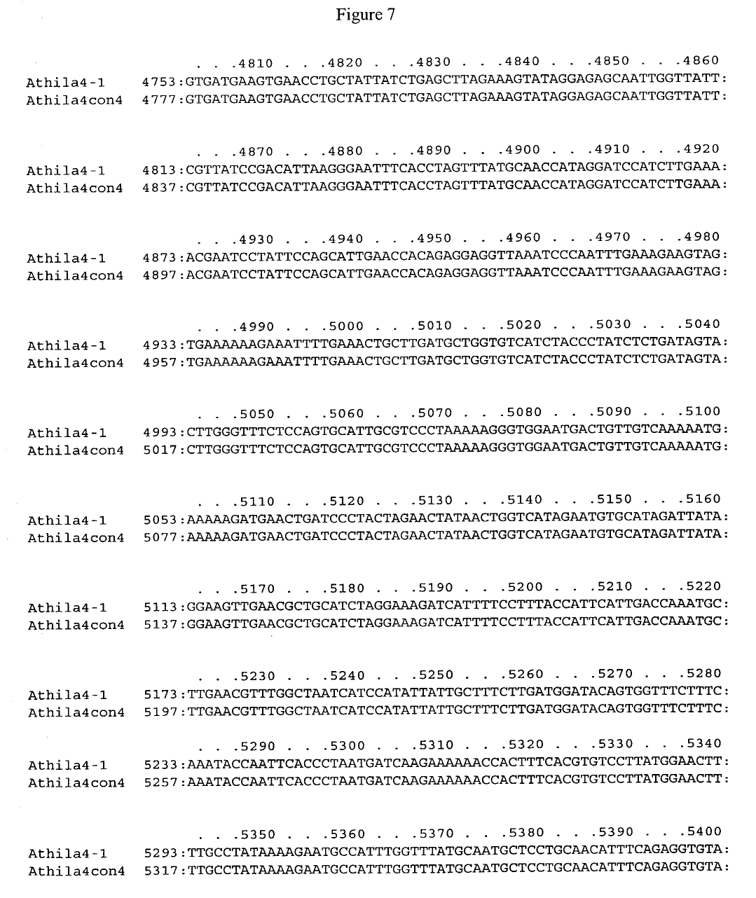

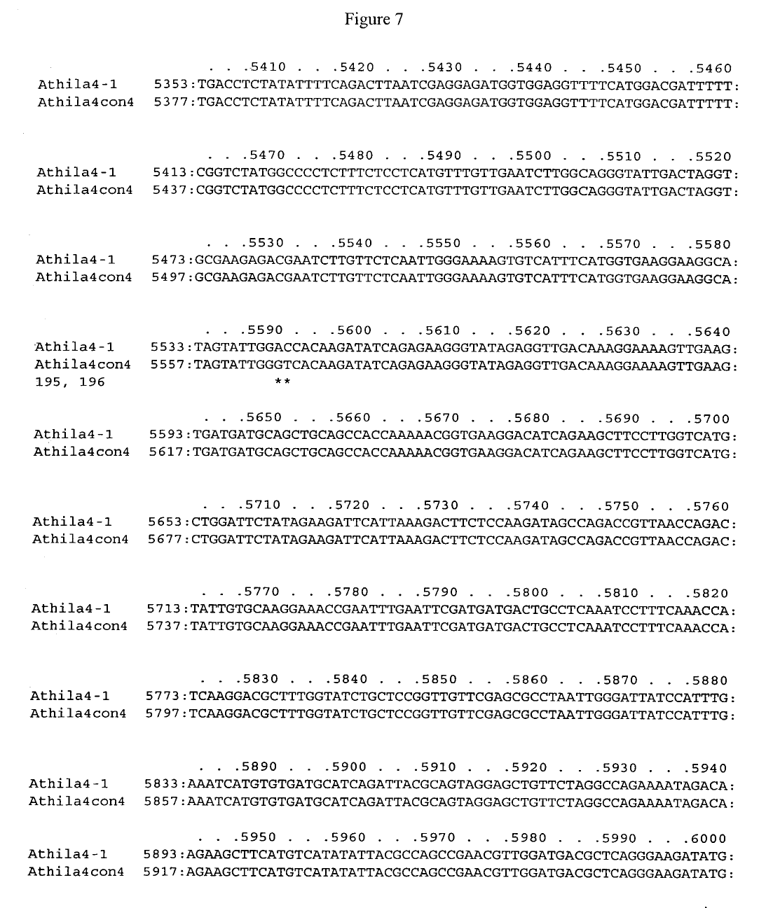

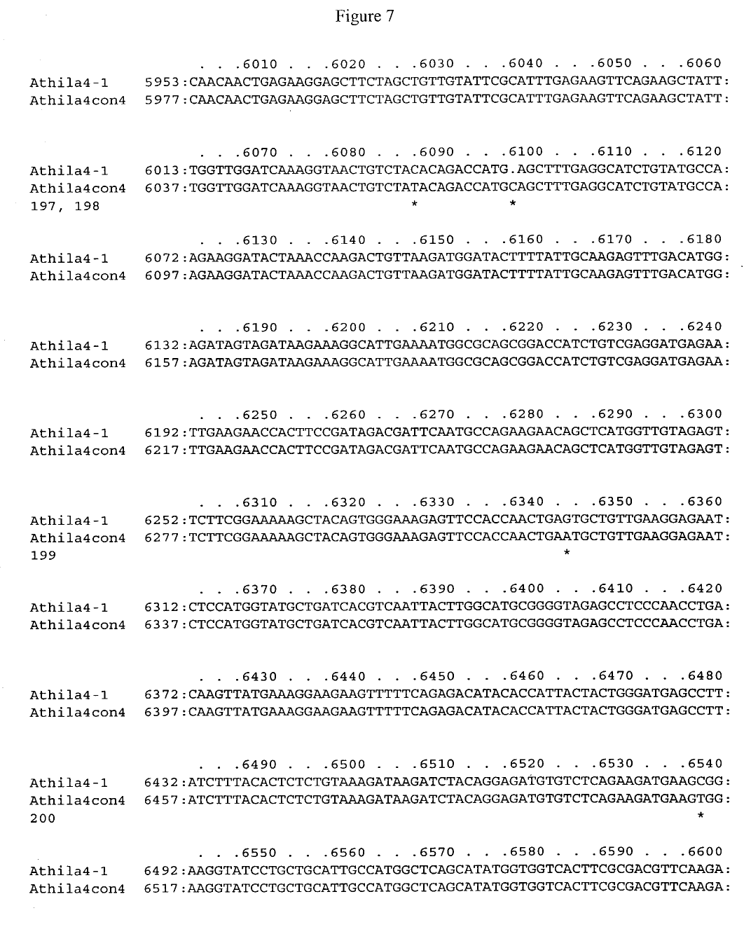

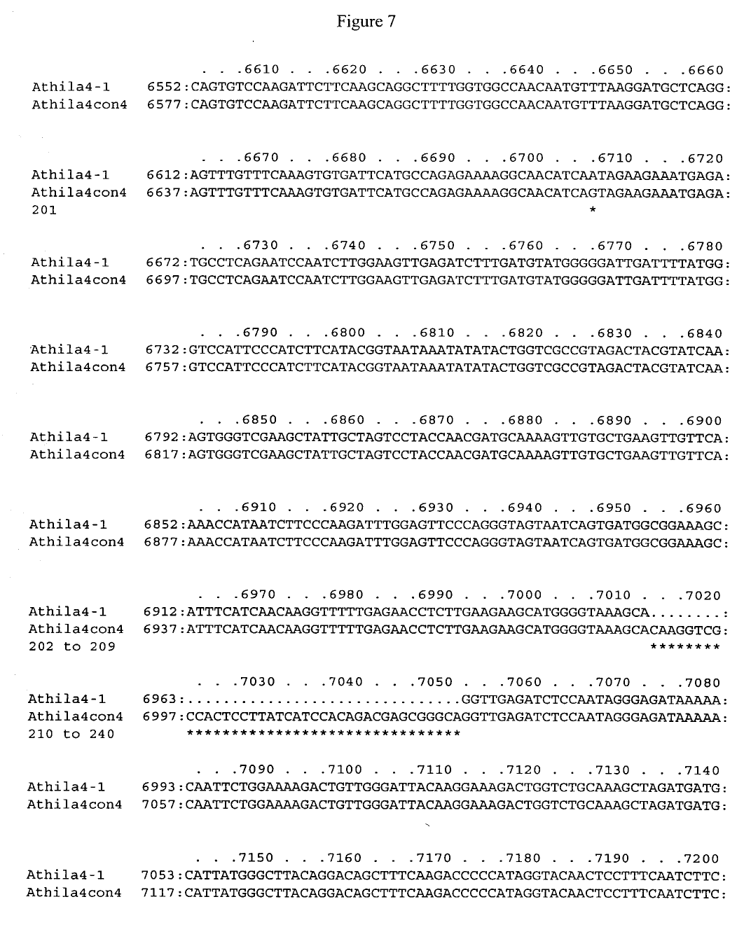

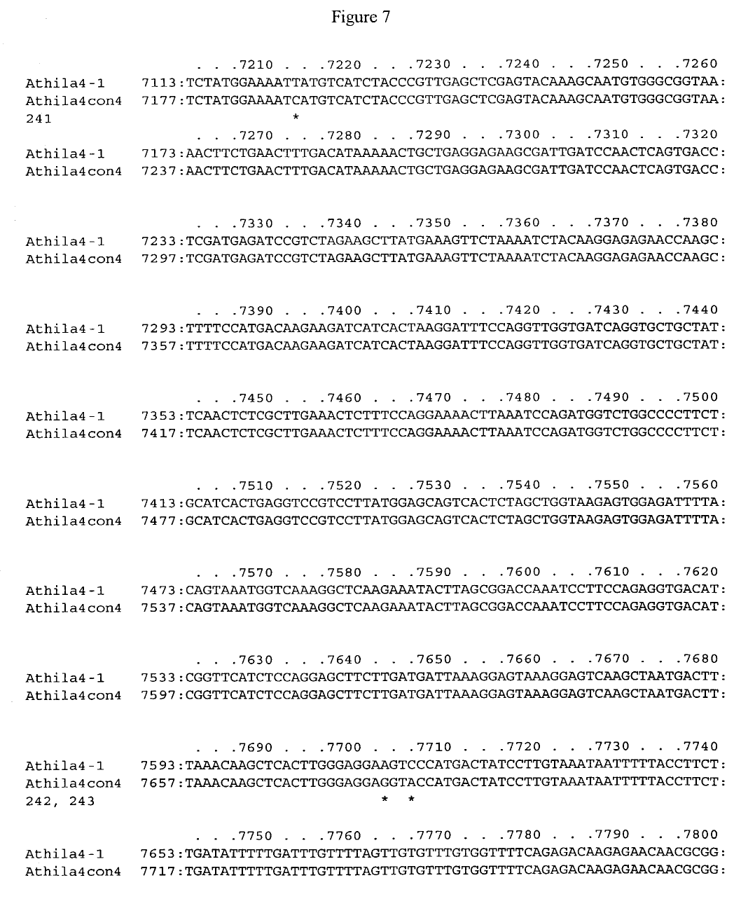

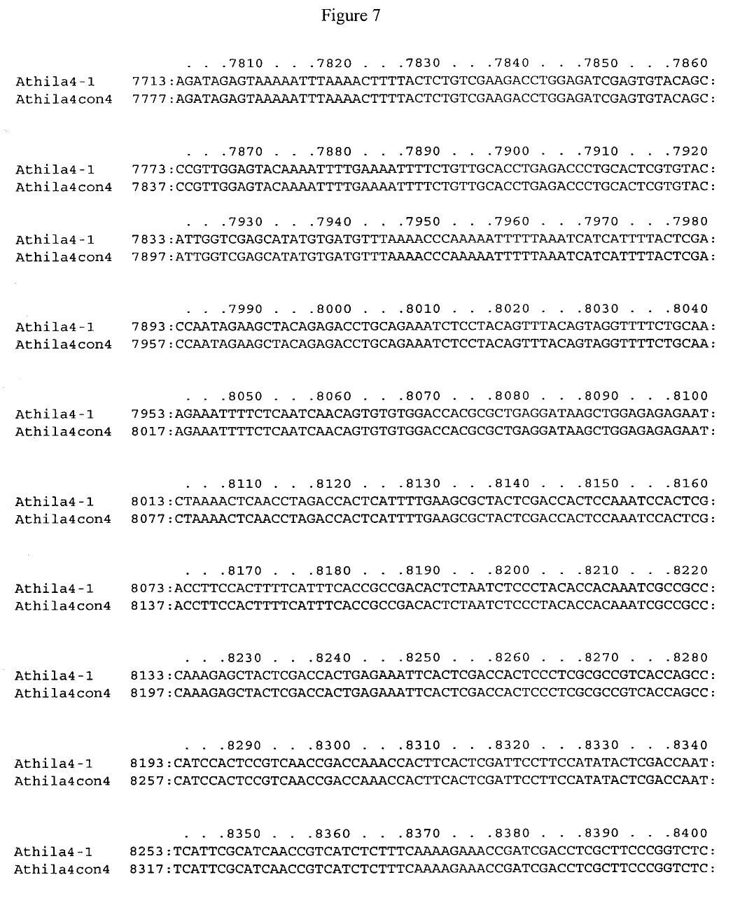

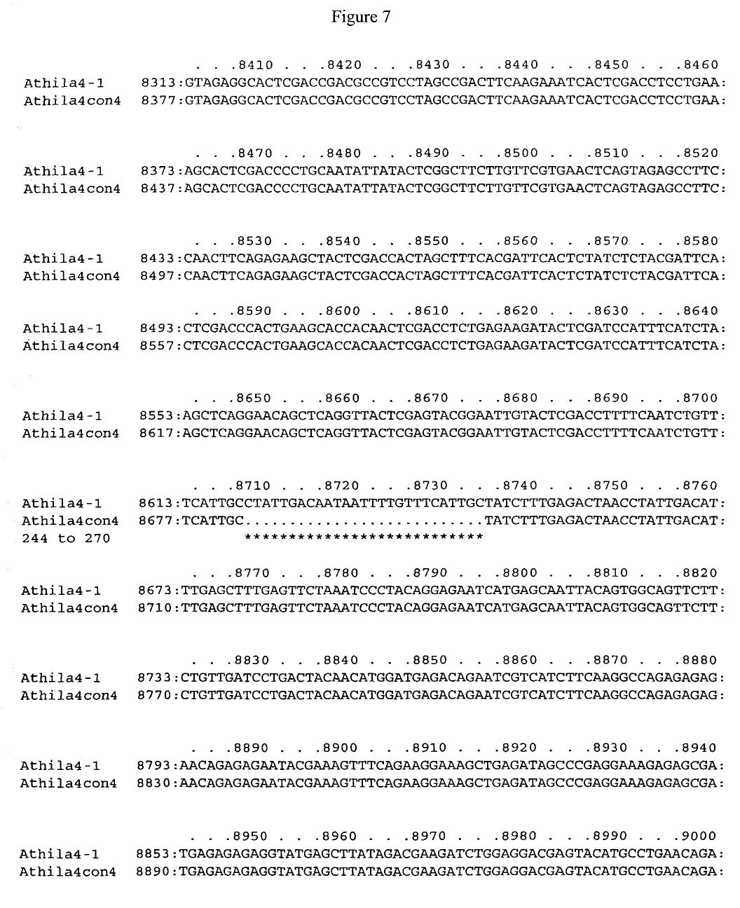

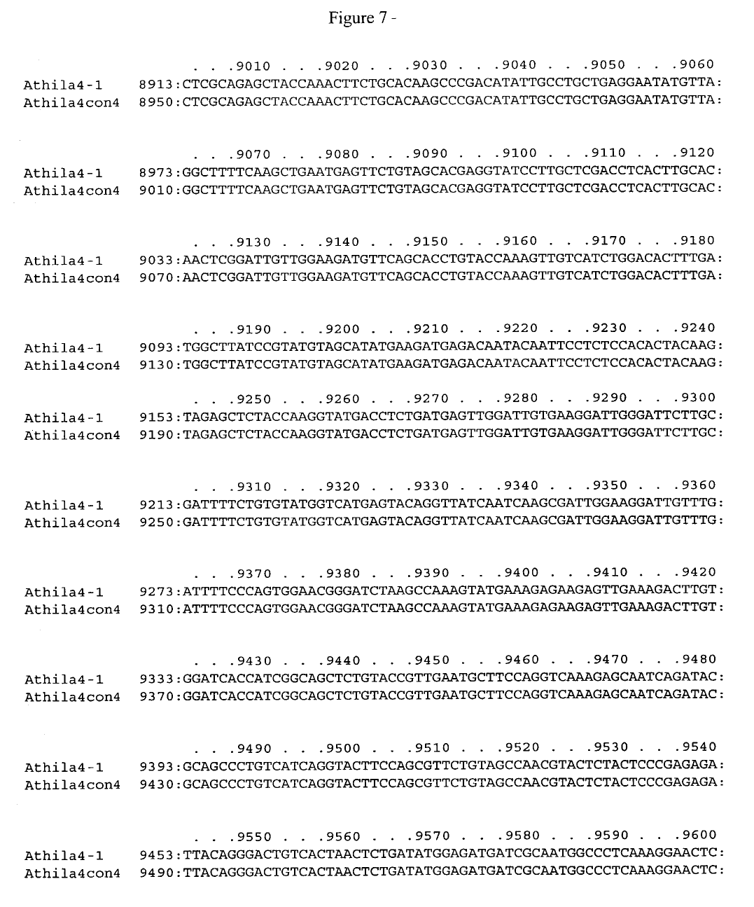

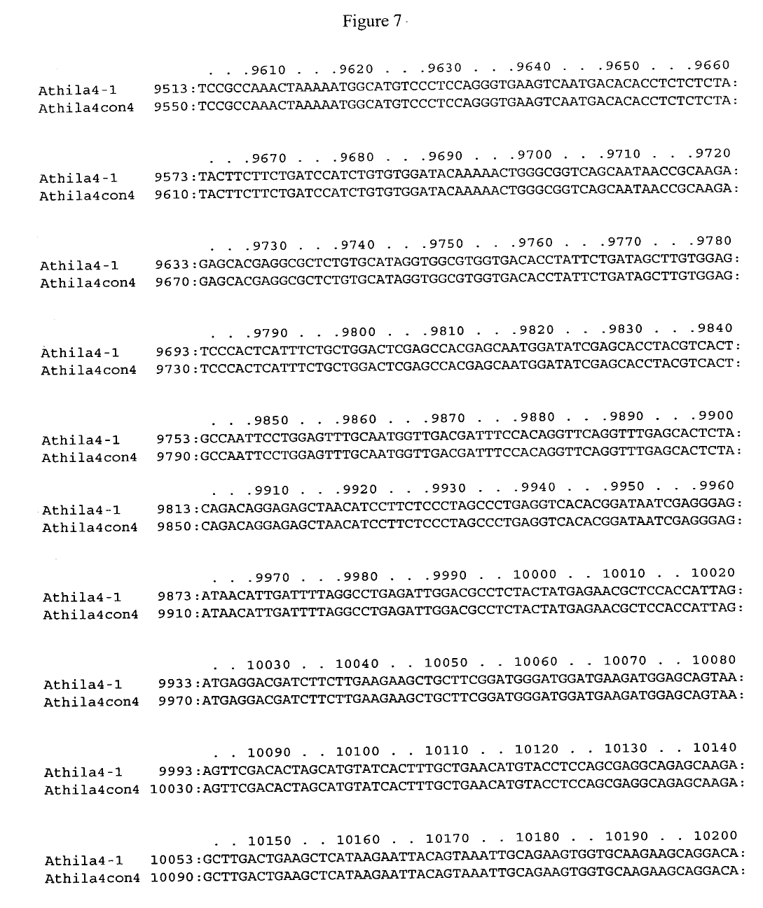

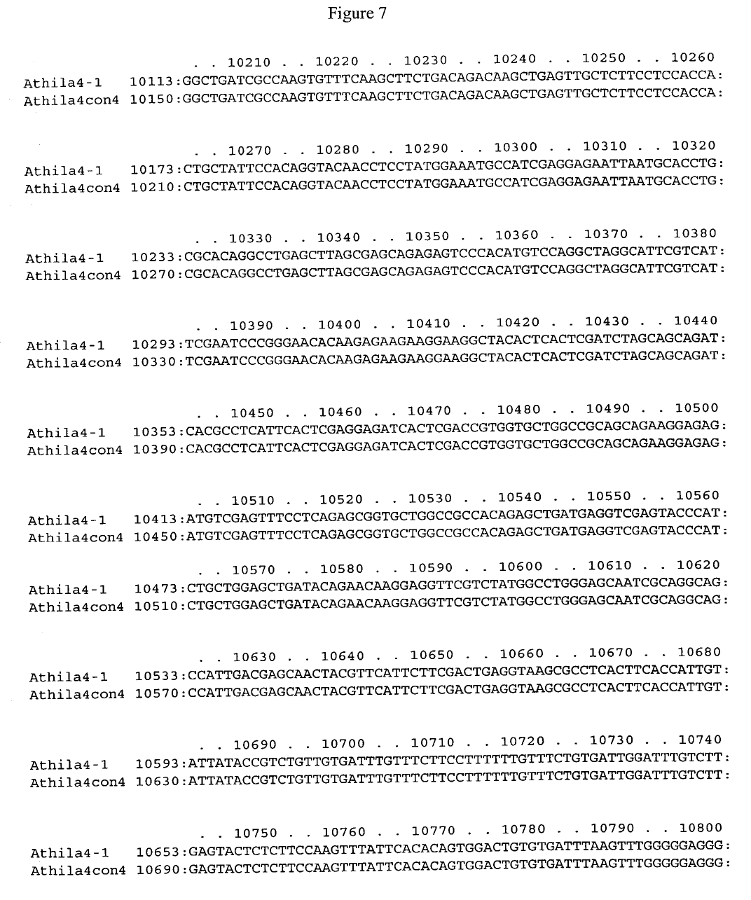

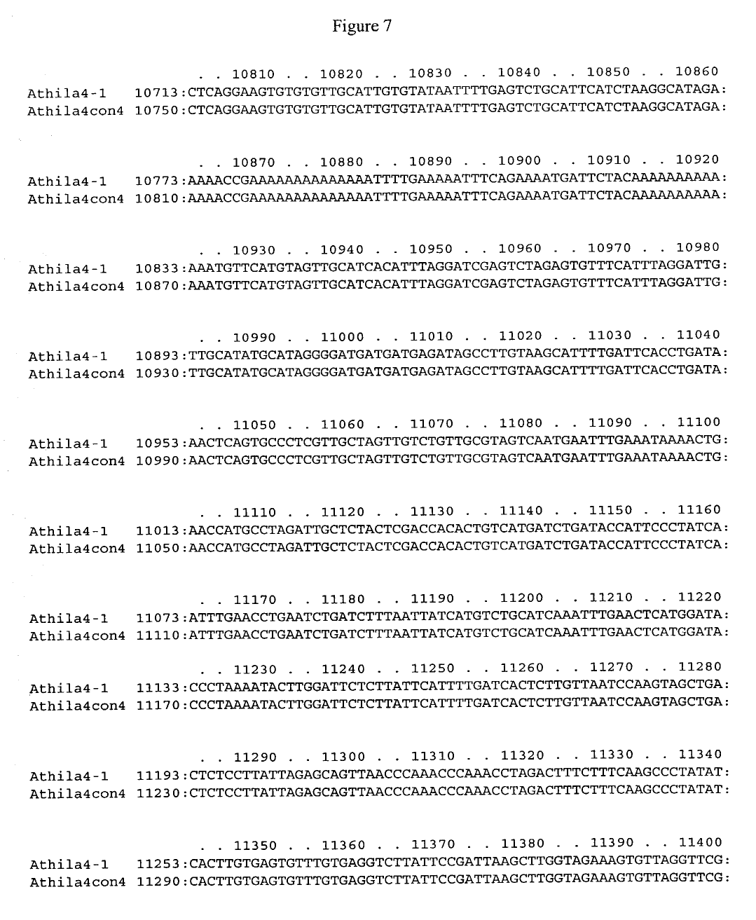

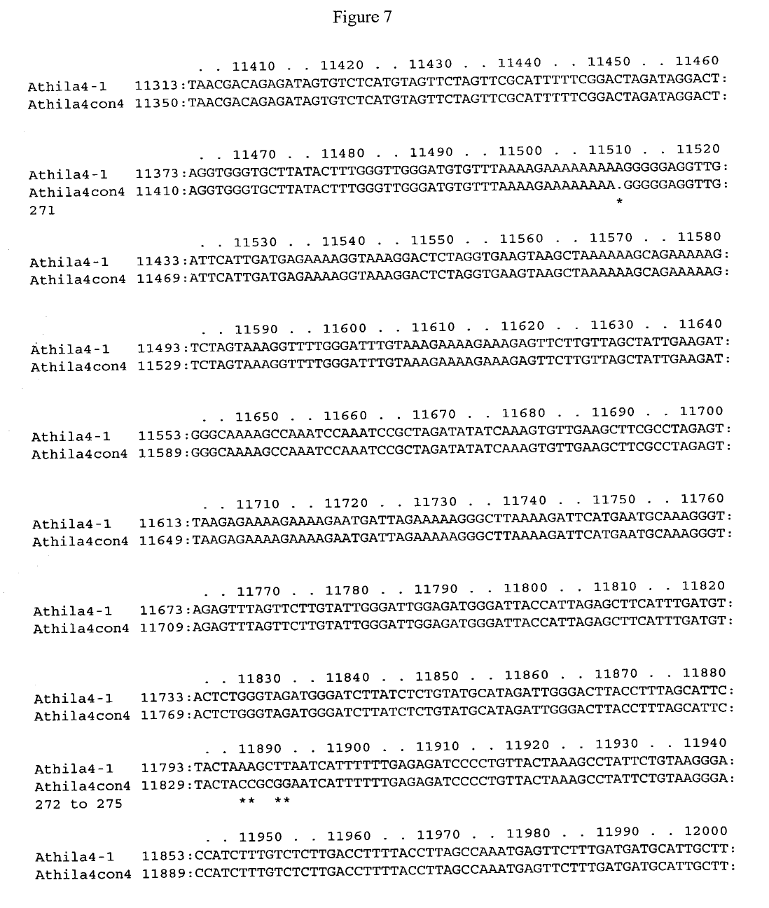

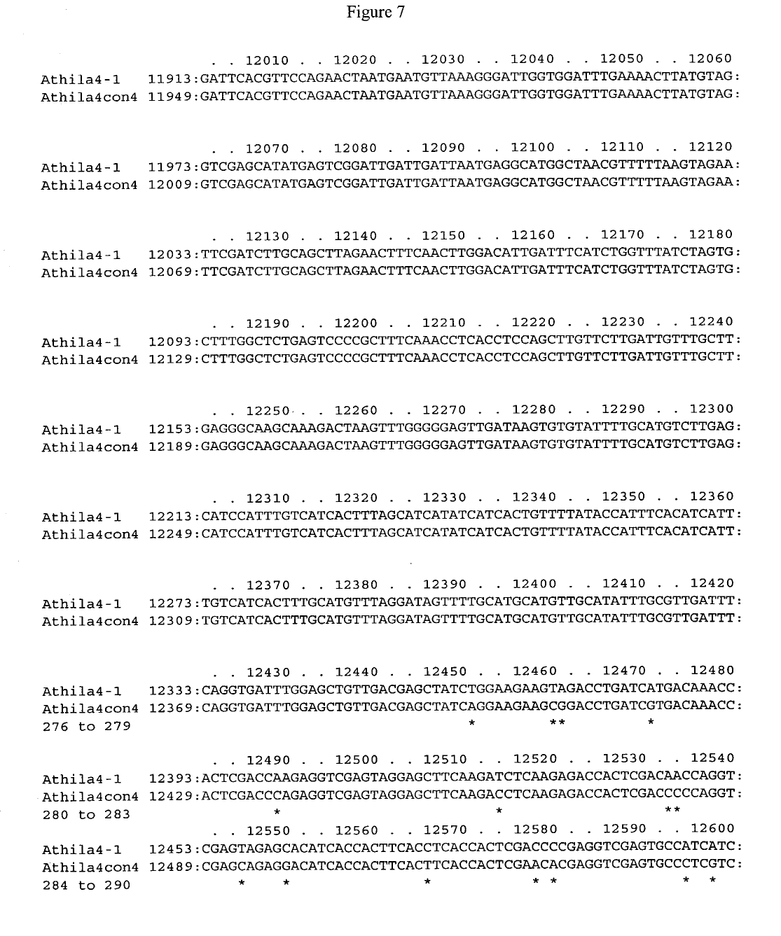

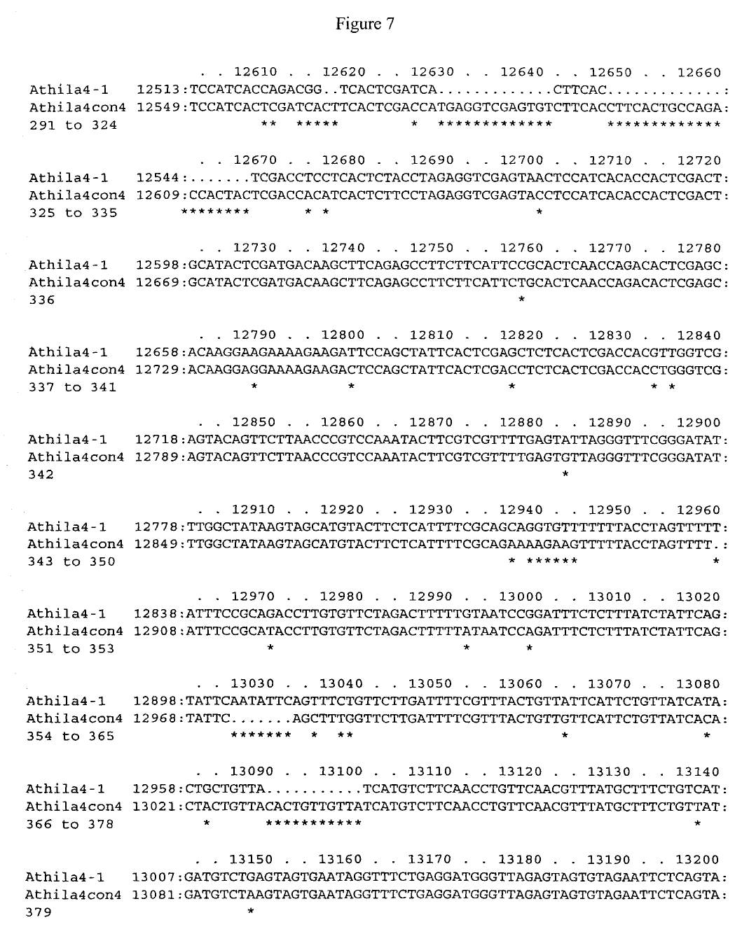

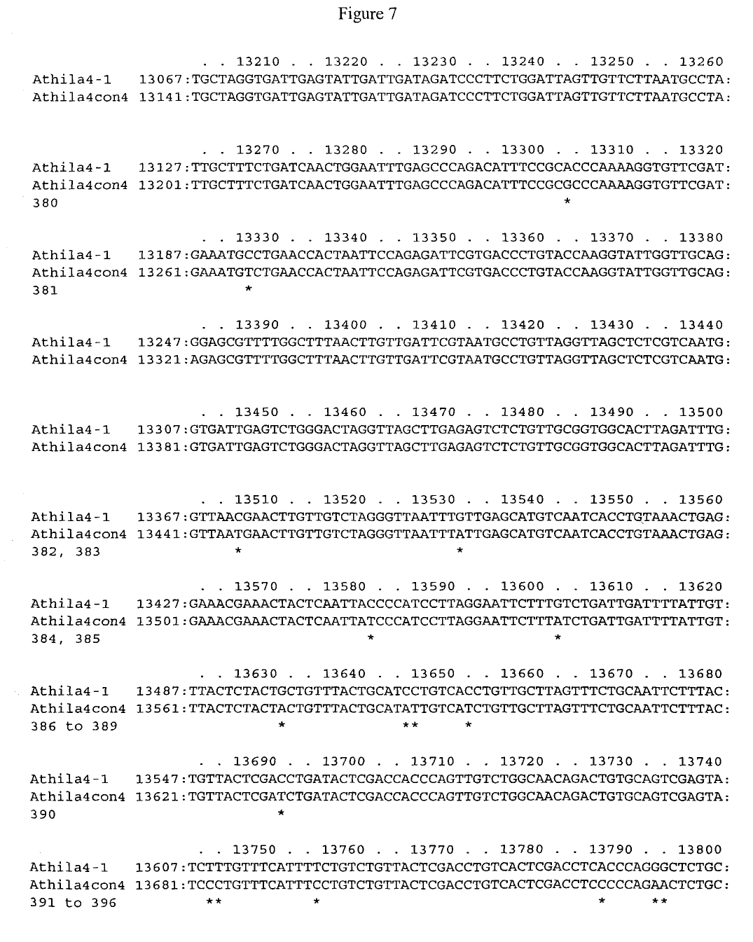

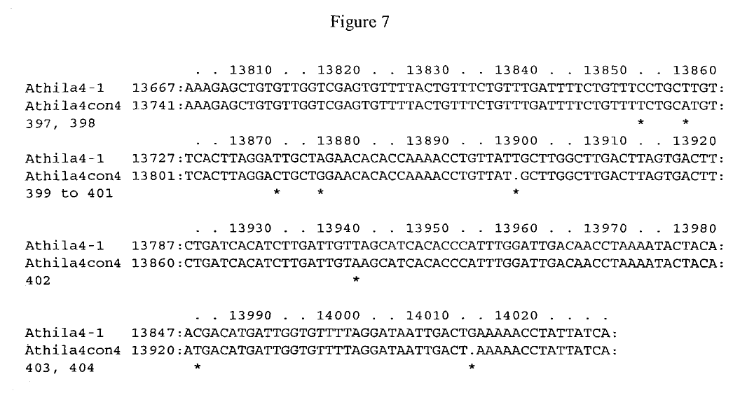

Consensus retroelements were constructed using sequential PCR site-directed mutagenesis (Ausubel et al. (1987) Current Protocols in Molecular Biology, Greene/Wiley Interscience (New York, N.Y.). Primers were synthesized that carry the desired nucleotide sequence changes (see below). FIG. 7 shows an alignment of a consensus nucleotide sequence with the Athila4-1 sequence. PCR products were generated in overlapping pairs, which were used in two rounds of amplification to create single PCR products with convenient terminal restriction sites. After cloning and sequencing, the PCR products were used to assemble the consensus retrovirus using standard cloning procedures. All PCR reactions were carried out using PFU polymerase and protocols supplied by Stratagene. The PCR reactions were performed in an MJ Research PC-100 PCR machine.

The changes that were introduced include the following: 1 to 108 result from a switch from the native Athila4-1 Long Terminal Repeat (LTR) to the related Athila4-6 LTR; 109 by PCR site directed mutagenesis using DVO1283 and DVO1284 resulted in an isoleucine to threonine amino acid change; 110 by PCR site directed mutagenesis using DVO1285 and DVO1286 resulted in a valine to alanine amino acid change; 111 by PCR site directed mutagenesis using DVO1285 and DVO1286 gave no amino acid change, but resulted in a nucleotide change to the consensus adenine; 112 by PCR site directed mutagenesis using DVO1287 and DVO1288 resulted in an asparagine to aspartic acid amino acid change; 113 by PCR site directed mutagenesis using DVO1289 and DVO1290 resulted in an asparagine to aspartic acid amino acid change; 114 by PCR site directed mutagenesis using DVO1108 and DVO1109 gave no amino acid change, but resulted in a nucleotide change to the consensus guanine; 115 by PCR site directed mutagenesis using DVO1108 and DVO1109. No amino acid change-resulted in a nucleotide change to the consensus cytosine; 116 by PCR site directed mutagenesis using DVO1108 and DVO1109 resulted in a proline to glutamine amino acid change; 117 by PCR site directed mutagenesis using DVO1108 and DVO1109. No amino acid change-resulted in a nucleotide change to the consensus thymine; 118 by PCR site directed mutagenesis using DVO1108 and DVO1109 resulted in the deletion of a proline amino acid; 119 by PCR site directed mutagenesis using DVO1108 and DVO1109 resulted in the deletion of a proline amino acid; 120 by PCR site directed mutagenesis using DVO1108 and DVO1109 resulted in the deletion of a proline amino acid; 121 by PCR site directed mutagenesis using DVO1108 and DVO1109 resulted in a serine to histidine amino acid change; 122 by PCR site directed mutagenesis using DVO1108 and DVO1109 resulted in a serine to histidine amino acid change; 123 by PCR site directed mutagenesis using DVO1108 and DVO1109 resulted in an alanine to proline amino acid change; 124 by PCR site directed mutagenesis using DVO1110 and DVO1111 resulted in an alanine to threonine amino acid change; 125 by PCR site directed mutagenesis using DVO1110 and DVO1111 resulted in a proline to threonine amino acid change; 126 by PCR site directed mutagenesis using DVO1112 and DVO1113 gave no amino acid change, but resulted in a nucleotide change to the consensus adenine; 127 by PCR site directed mutagenesis using DVO1112 and DVO1113 resulted in an asparagine to lysine amino acid change; 128 by PCR site directed mutagenesis using DVO1146 and DVO1147 gave no amino acid change, but resulted in a nucleotide change to adenine to stabilize a repeated DNA region; 129 by PCR site directed mutagenesis using DVO1147 and DVO1162 resulted in a glutamine to leucine amino acid change; 130 by PCR site directed mutagenesis using DVO1147 and DVO1162 resulted in a proline to serine amino acid change; 131 by PCR site directed mutagenesis using DVO1147 and DVO1162 gave no amino acid change, but resulted in a nucleotide change to adenine to stabilize a repeated DNA region; 132 by PCR site directed mutagenesis using DVO1147 and DVO1162 gave no amino acid change, but resulted in a nucleotide change to guanine to stabilize a repeated DNA region; 133 to 168 by PCR site directed mutagenesis using DVO1147, DVO1148, DVO1162 and DVO1163 resulted in an insertion of the nucleotides TTTGGATTTAAGTCTTCAGCAATCATTGGACCCGCC (SEQ ID NO:160), which added the amino acid sequence PLDLSLQQSLDP (SEQ ID NO:161), which is a repeat in the amino acid sequence and variations are found in related plant retroviruses; 169 to 182 by PCR site directed mutagenesis using DVO1148, DVO1149, DVO1163 and DVO1164 gave no amino acid changes, but resulted in nucleotide changes to stabilize a repeated DNA region; 183 by PCR site directed mutagenesis using DVO1149 and DVO1164 resulted in an arginine to lysine amino acid change; 184 by PCR site directed mutagenesis using DVO1149 and DVO1164 resulted in an arginine to lysine amino acid change; 185 by PCR site directed mutagenesis using DVO1149 and DVO1150 gave no amino acid changes, but resulted in nucleotide change to a consensus guanine; 186 by PCR site directed mutagenesis using DVO985 and DVO986 resulted in addition of a thymine, which caused a frame shift correction and the codon was changed from TTC to TTT (both of which code for phenylalanine); 187 by PCR template choice with DVO986 and DVO1116 gave no amino acid changes; 188 by PCR template choice with DVO986 and DVO1116 gave no amino acid changes; 189 by PCR template choice with DVO986 and DVO1116 gave no amino acid changes; 190 by PCR template choice with DVO1117 and DVO1118 resulted in a histidine to proline amino acid change; 191 by PCR template choice with DVO1117 and DVO1118 gave no amino acid changes; 192 by PCR template choice with DVO1117 and DVO1118 gave no amino acid changes; 193 by PCR template choice with DVO1117 and DVO1118 gave no amino acid changes; 194 by PCR template choice with DVO1117 and DVO1118 resulted in an isoleucine to methionine amino acid change; 195 by PCR site directed mutagenesis using DVO1272 and DVO1273 resulted in an aspartic acid to glycine amino acid change; 196 by PCR site directed mutagenesis using DVO1272 and DVO1273 resulted in an aspartic acid to glycine amino acid change; 197 by PCR site directed mutagenesis using DVO1274 and DVO1275 gave no amino acid changes, but resulted in nucleotide change to a consensus thymine; 198 by PCR site directed mutagenesis using DVO1274 and DVO1275 resulted in addition of a cytosine, which caused a frame shift correction and the codon was changed from GAG to GCA, adding an alanine to the amino acid sequence; 199 by PCR site directed mutagenesis using DVO1276 and DVO1277 resulted in a serine to asparagine amino acid change; 200 by PCR site directed mutagenesis using DVO1278 and DVO1279 resulted in an alanine to valine amino acid change; 201 by PCR site directed mutagenesis using DVO1280 and DVO1281 resulted in an asparagine to serine amino acid change; 202 to 240 by PCR site directed mutagenesis using DVO1023 and DVO124 to correct a deletion, resulting in an insertion of the nucleotides CAAGGTCGCCACTCCTTATCATCCACAGACGAGCGGGCA (SEQ ID NO:162, which added the amino acid sequence HKVATPYHPQTSG (SEQ ID NO:163); 241 and 242 by PCR site directed mutagenesis using DVO1024 and DVO1282 resulted in a leucine to serine amino acid change; 243 by PCR site directed mutagenesis using DVO1291 and DVO1294 to create a unique KpnI restriction endonuclease cloning site to facilitate DNA manipulation; 243 to 270 by deletion of an unstable non-coding DNA fragment in E. coli—this fragment is between two small DNA repeats that apparently recombine in E. coli at a high frequency, resulting in this common 27 base deletion in the plant retroelement clones; 271 by deletion in E. coli—this region contains a series of adenines and by chance or by an instability, one of the adenines was deleted, although this deletion is not predicted to have an effect on the plant retrovirus clone; 272 to 275 by PCR site directed mutagenesis using DVO993 and DVO994 to create a unique SacII restriction endonuclease cloning site; and 276 to 404 result from a switch from the native Athila4-1 LTR to the related Athila4-6 LTR.

For the initial element (pDW739), the consensus gag/pol coding region was based on sequence alignment data for Athila4-1 and Athila4-2. The 5′ LTR of the Athila4-1 element was used for both the 5′ and 3′ LTRs of pDW739. By the time the first construct was finished, Athila4-3 and Athila4-4 elements had been identified in the A. thaliana genome sequence. The sequence data for the new elements suggested changes that were incorporated into a revised consensus element (pDW762). When this construct was completed, Athila4-5 and Athila4-6 were found and added to the consensus. The Athila4-5 sequence indicated that a few sequence changes could be made to refine the consensus, but the addition of Athila4-6 added no new information. This suggests that a true consensus had been achieved. These changes were incorporated into a new consensus sequence (SEQ ID NO:122), which uses the 5′ LTR from Athila4-6 for the 5′ and 3′ LTRs. FIG. 8 shows a nucleotide alignment of all Athila4 elements used to generate the consensus. Included in the alignment is the sequence of the consensus element. FIG. 9 shows the nucleotide sequence of the consensus element, along with translations of its coding regions. FIG. 10 shows an alignment of the Gag-Pol amino acid sequence of all Athila4 elements used to generate the consensus. Included is the amino acid sequence of the coding region of the consensus element.

### Example 4

### The Consensus Element Encodes a Functional Reverse Transcriptase

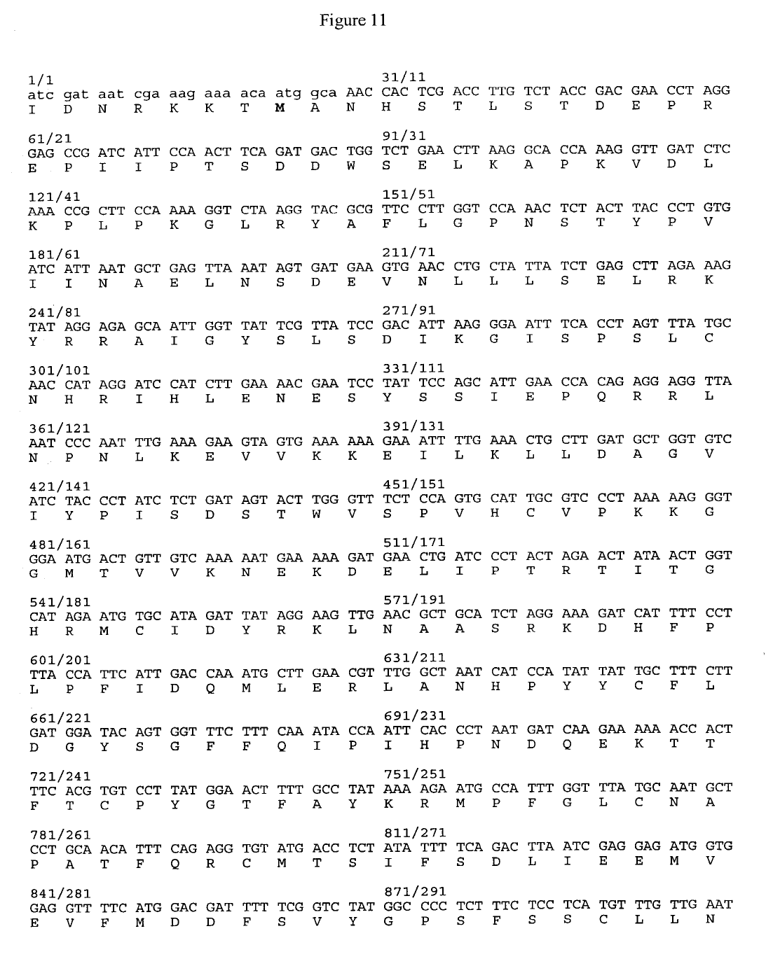

The approximate boundaries of reverse transcriptase were determined by comparative sequence analysis of closely related plant retroelements, namely the Athila 1, Athila4 and Athila6 elements, Cyclops from pea, Calypso from soybean, Bagy2 from barley and an unnamed plant retrovirus from rice. To produce a functional reverse transcriptase, 4 nucleotide changes were made to the reverse transcriptase clone (pJR3) by site directed PCR mutagenesis (see FIG. 11 for details). Two changes (adenine to guanine and cytosine to thymine respectively) that correspond to alterations 195 and 196 on the Athila4 modification map (FIG. 11) resulted in an aspartic acid to a conserved glycine substitution. A conserved nucleotide change (cytosine to thymine) was made for correction 197, but it did not result in an amino acid change. Additionally, a frame shift mutation was corrected, which corresponds to change 198 on the Athila4 modification map (FIG. 11). A cytosine was added to correct the frameshift, which resulted in a codon change from GAG to GCA; this added an alanine to the amino acid sequence. The beginning sequence 5′-atcgataatcgaaagaaaacaatggca (lowercase nucleotide sequence, SEQ ID NO:164) was added to give the clone a convenient 5′ cloning site (ClaI) and a signal that includes a translation start site. The end sequence 5′-atggaacaaaagcttatctctgaagaggatcttggttgataataggagctc (lowercase nucleotide sequence, SEQ ID NO:165) was added to give the clone a convenient 3′ restriction endonuclease cloning site (SacI), an epitope tag (C-myc) for subsequent protein identification, and a series of stop codons to signal translation termination. The stop codon signals are represented by Z.

A consensus reverse transcriptase was produced in vitro and tested for enzymatic activity. The RT protein was prepared by synthesizing capped RNA from a DNA template (pJR3) using Ambion's mMessage mMachine transcription kit. The purified RNA was then translated with Ambion's Wheat Germ IVT kit. Upon completion of translation the reaction was centrifuged at 20,000×g, 4° C., for 2 minutes. The crude supernatant was transferred into a new microcentrifuge tube on ice and assayed for activity.

The RT assay was based on a method by Wilhelm et al. 2000 (Biochem. J. 348: 337-342). The crude translation supernatant, with or without Athila4 RT (7.9 ul), was tested in triplicate for RT activity and compared to 5 units of AMV RT. Activity was measured by following the poly(rA)ₙ-oligo(dT)₁₂₋₁₈ directed incorporation of [α-³²P]dTTP. For a negative control the supernatants were boiled for 3 minutes prior to assaying. The assay mix (20 μl) contained 50 mM Tris/HCl, pH 8.0, 15 mM NaCl, 20 mM MgCl₂, 0.15 μM dTTP, 8 mM 2-mercaptoethanol, 0.01 unit of poly(rA)ₙ-oligo(dT)₁₂₋₁₈ and 1 μCi of [α-³²P]dTTP. Reactions were incubated for 60 min at 22° C. Incorporation of ³²P-radiolabelled dTTP was determined by spotting 9 μl of the reaction onto both DE-81 and GF/C paper. The DE-81 paper was washed 3×20 minutes in 2×SSC, and once in 100% ethanol for 1 minute to remove any unincorporated ³²P-radiolabelled dTTP. The GF/C paper was not washed and was used to determine total (incorporated and unincorporated) ³²P-dTTP in the reactions. The filter papers were allowed to air dry and the amount of radioactivity was measured on a scintillation counter to determine the average counts per minute (CPM).

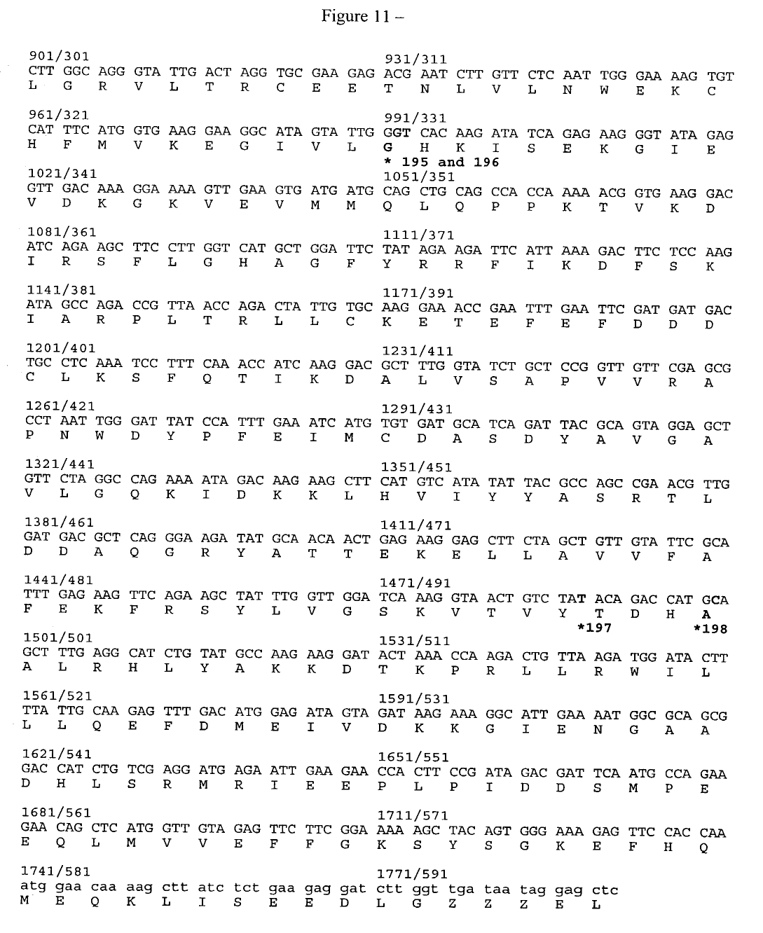

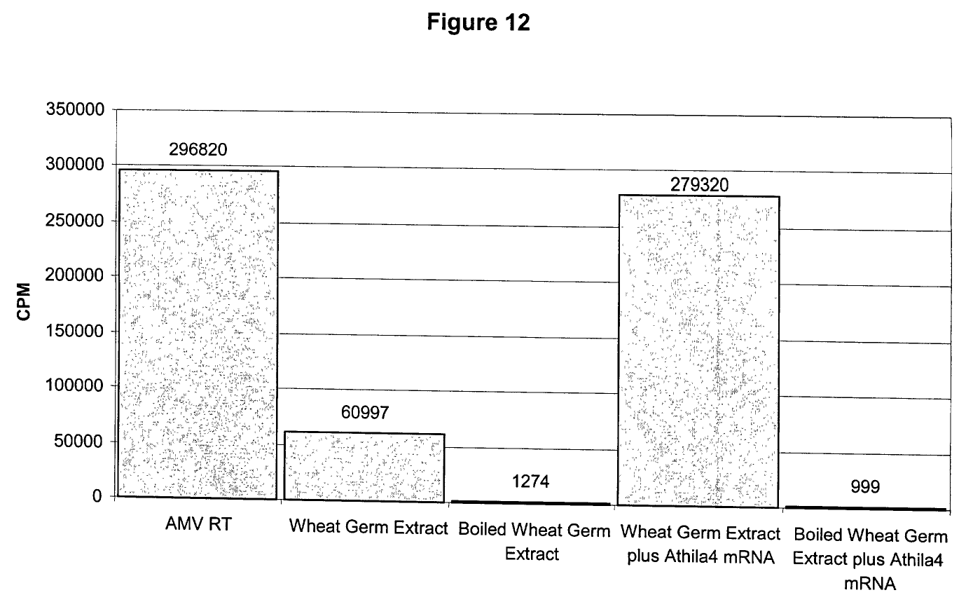

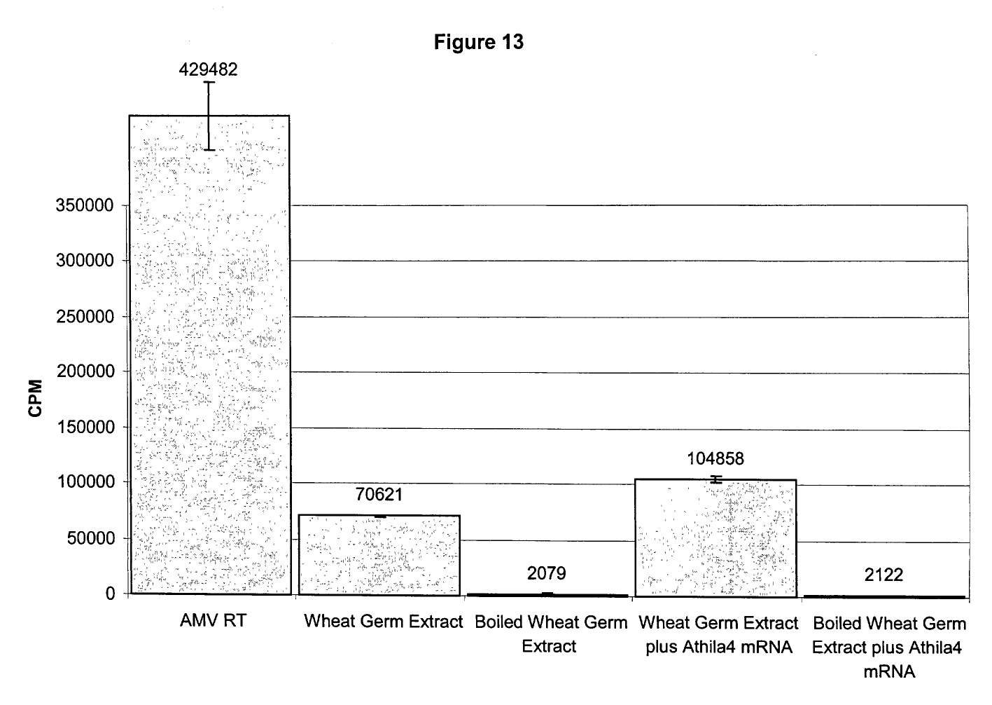

The crude translation reactions containing consensus RT consistently yielded 1.4 to 4.5 times more activity than those reactions without consensus RT. In addition, boiling the crude translation reaction containing consensus RT prior to conducting the enzymatic RT assay destroyed all activity. When compared to the activity of a purified and commercially available RT (5 units of Avian Myeloblastosis Virus (AMV) RT), consensus RT was found to have from 4 times less to equivalent levels of activity (FIGS. 12 and 13). These data collectively indicate that the consensus retroelement produces a functional RT.

### Example 5

### The Consensus Retroelement Encodes a Functional Protease

Retroelements express a Gag-Pol polyprotein that is cleaved by an element-encoded protease. Products of this cleavage reaction are Gag, PR, RT, and IN. Comparative sequence analyses were conducted to determine the approximate boundaries of protease and potential protease cleavage sites within the consensus element. Gag-Pol amino acid sequences were aligned for several closely related plant retroelements, namely the Athila1, Athila4 and Athila6 elements, Cyclops from pea, Calypso from soybean, Bagy2 from barley and an unnamed plant retrovirus from rice. From these sequence alignments, the consensus retroelement protein domains were defined as follows (capital letters are conserved sequences and small letters are potential sites of protease cleavage): Gag/PR, TEDSEDQDGEDlslekdqadkpldlsleqpldlslqqsldppldsitrpttrpvipaasptapkpvavknkekVFVPPPYKP (SEQ ID NO:166); PR/RT, LLDSHKAMEESEPFEELNGPATEVMVMSEegstrvqpalsrtyssnhstlstdeprepiiptsd DWSELKAP (SEQ ID NO:167); and RT/IN, SMPEEQLMVVeffgksysgkefhqlnavegesPWYADHVNYLAC (SEQ ID NO:168).

To test whether the consensus retroelement has protease activity, a series of constructs were made that express all or part of gag-pol. These constructs used a heterologous promoter (the cauliflower mosaic virus 35S promoter) and a heterologous terminator (nopaline synthase). To detect consensus proteins, a c-myc epitope tag was added to the N-terminus of Gag between the first methionine and the sequence arginine-threonine-arginine-serine (the epitope sequence is EQKLISEEDLG; SEQ ID NO:169). The initial construct (pDW836) expresses the complete gag-pol. Deletions were made in pDW836 using convenient restriction sites within gag-pol and an Acc65 I site at the end of the coding region. These digestion products were treated with mung bean nuclease and self-ligated to create pDW1018 and pDW1035 to pDW1038 (see Table 5).

Each of the six constructs was transiently expressed in freshly prepared tobacco SRI protoplasts by electroporation. After approximately 24 hours, the protoplasts were collected and prepared for Western analysis by centrifuging a 5 ml sample at 100×g for 10 minutes. The supernatant was removed and the pellet was resuspended with 100 μl of 2×SDS loading buffer and heated to 80° C. for 10 minutes. The sample was either stored at −80° C. or prepared for electrophoresis by centrifugation at 14,000×g for 3 minutes. A 40 μl aliquot of supernatant from each sample was subjected to electrophoresis at 200 volts on an 8% SDS-PAGE gel and then electrophoretically transferred overnight at 4° C. and 100 mAmp to nitrocellulose. After transfer, the nitrocellulose was blocked with 10% (wt/vol) non-fat dry milk in TBS-Tween-Triton (TBSTT) (10 mM Tris-HCl [pH 7.5], 150 mM NaCl, 0.05% Tween 20, 0.2% Triton X-100) for 1 hour. The nitrocellulose was then treated as follows: A) incubated 1 hour with a c-Myc 9E10 monoclonal antibody (Santa Cruz) that had been diluted 1:200 in blocking buffer; B) washed 4 times for 5 minutes each in blocking buffer; C) incubated 1 hour with a horseradish peroxidase-conjugated goat-anti-mouse antibody (Santa Cruz) diluted 1:3000 in blocking buffer; D) washed 4 times for 15 minutes each in TBSTT; E) developed with ECL (Amersham) and exposed to film.

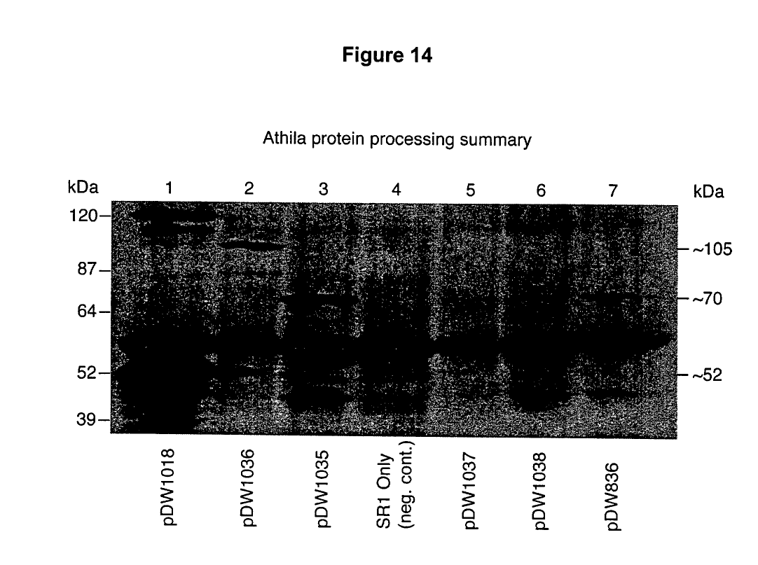

Two potential outcomes were predicted for the western blot experiments: 1) if consensus protease was not active, a protein would be detected corresponding in size to the length of the expressed open reading frame; 2) alternatively, if consensus protease was active, a Gag protein of approximately 58.8 to 65.5 kDa would be detected that is released from the full-length protein. pDW1035 encodes a 76.7 kDa protein (this construct does not contain a complete protease) and an approximately 70 kDa protein was detected (FIG. 14). pDW1036 encodes a 111.2 kDa protein; it is predicted to encode a complete protease and therefore should produce a 58.8 to 65.5 kDa Gag protein. However, the observed 110 kDa protein indicates that either protease is not active or is unable to cleave this protein. pDW1037, pDW1038 and pDW836 each encode a complete protease and each produced a 70 kDa protein. This is slightly larger than the predicted size of Gag, and this may be the consequence of posttranslational modification. Nonetheless, the data collectively demonstrate that the consensus retroelement encodes a functional protease that is capable of cleaving the polyprotein.

TABLE 5
Determining protease activity of the consensus elements.

| | | Expected | Expected | Observed |
|---|---|---|---|---|
| | | Amino | Molecular | Molecular |
| | | Acid | Weight | Weight |
| Construct | Deleted Region | length | (kDa) | (kDa) |
| PDW1018 | BstE II to Acc65 I | 336 | 38.5 | 52 and 120 |
| PDW1035 | BspH I to Acc65 I | 680 | 76.7 | 70 |
| PDW1036 | Mlu I to Acc65 I | 988 | 111.2 | 110 |
| PDW1037 | Hpa I to Acc65 I | 1324 | 149.7 | 70 |
| PDW1038 | Sph I to Acc65 I | 1534 | 171.4 | 70 |
| PDW836 | NA | 1922 | 218.3 | 70 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed:

1. An isolated retroelement comprising a nucleotide sequence that is at least 90% identical to the nucleotide sequence set forth in SEQ ID NO:122, or the complement thereof.

2. An isolated nucleic acid encoding a polypeptide, wherein said polypeptide comprises an amino acid sequence that is at least 85% identical to the amino acid sequence set forth in SEQ ID NO:128.

3. An isolated nucleic acid comprising a nucleotide sequence that is at least 90% identical to nucleotides 1 to 1747 or 12220 to 13966 of the sequence set forth in SEQ ID NO:122, or the complement thereof.

4. The isolated nucleic acid of claim 3, wherein said nucleotide sequence is at least 95% identical to nucleotides 1 to 1747 or 12220 to 13966 of the sequence set forth in SEQ ID NO:122, or the complement thereof.

5. The isolated nucleic acid of claim 3, wherein said nucleotide sequence is at least 98% identical to nucleotides 1 to 1747 or 12220 to 13966 of the sequence set forth in SEQ ID NO:122, or the complement thereof.

6. An isolated nucleic acid comprising a nucleotide sequence that is at least 90% identical to nucleotides 1 to 385 of the sequence set forth in SEQ ID NO:122, or the complement thereof.

7. An isolated nucleic acid comprising a nucleotide sequence that is at least 90% identical to nucleotides 1 to 40 or 1708 to 1747 of the sequence set forth in SEQ ID NO:122, or the complement thereof.

8. An isolated nucleic acid encoding a polypeptide, wherein said polypeptide comprises an amino acid sequence that is at least 85% identical to the amino acid sequence set forth in SEQ ID NO:140.

9. An isolated nucleic acid comprising a nucleotide sequence that is at least 90% identical to nucleotides 1893 to 3575 of the sequence set forth in SEQ ID NO:122, or the complement thereof.

10. An isolated nucleic acid encoding a polypeptide, wherein said polypeptide comprises an amino acid sequence that is at least 85% identical to the amino acid sequence set forth in SEQ ID NO:141.

11. The isolated nucleic acid of claim 10, wherein said amino acid sequence is at least 90% identical to the amino acid sequence set forth in SEQ ID NO:141.

12. The isolated nucleic acid of claim 10, wherein said amino acid sequence is at least 95% identical to the amino acid sequence set forth in SEQ ID NO:141.

13. An isolated nucleic acid comprising a nucleotide sequence that is at least 90% identical to nucleotides 3576 to 4556 of the sequence set forth in SEQ ID NO:122, or the complement thereof.

14. The isolated nucleic acid of claim 13, wherein said nucleotide sequence is at least 95% identical to nucleotides 3576 to 4556 of the sequence set forth in SEQ ID NO:122, or the complement thereof.

15. The isolated nucleic acid of claim 13, wherein said nucleotide sequence is at least 98% identical to nucleotides 3576 to 4556 of the sequence set forth in SEQ ID NO:122, or the complement thereof.

16. An isolated nucleic acid encoding a polypeptide, wherein said polypeptide comprises an amino acid sequence that is at least 85% identical to the amino acid sequence set forth in SEQ ID NO:139.

17. The isolated nucleic acid of claim 16, wherein said amino acid sequence is at least 90% identical to the amino acid sequence set forth in SEQ ID NO:139.

18. The isolated nucleic acid of claim 16, wherein said amino acid sequence is at least 95% identical to the amino acid sequence set forth in SEQ ID NO:139.

19. An isolated nucleic acid comprising a nucleotide sequence that is at least 90% identical to nucleotides 4602 to 6314 of the sequence set forth in SEQ ID NO:122, or the complement thereof.

20. The isolated nucleic acid of claim 19, wherein said nucleotide sequence is at least 95% identical to nucleotides 4602 to 6314 of the sequence set forth in SEQ ID NO:122, or the complement thereof.

21. The isolated nucleic acid of claim 19, wherein said nucleotide sequence is at least 98% identical to nucleotides 4602 to 6314 of the sequence set forth in SEQ ID NO:122, or the complement thereof.

22. An isolated nucleic acid encoding a polypeptide, wherein said polypeptide comprises an amino acid sequence that is at least 85% identical to the amino acid sequence set forth in SEQ ID NO:142.

23. An isolated nucleic acid comprising a nucleotide sequence that is at least 90% identical to nucleotides 6315 to 7625 of the sequence set forth in SEQ ID NO:122, or the complement thereof.

24. An isolated nucleic acid encoding a polypeptide, wherein said polypeptide comprises an amino acid sequence that is at least 85% identical to the amino acid sequence set forth in SEQ ID NO:129, SEQ ID NO:130, or SEQ ID NO:131.

25. An isolated nucleic acid comprising a nucleotide sequence that is at least 90% identical to nucleotides 8745 to 10600, nucleotides 8745 to 10673, or nucleotides 8745 to 10728 of the sequence set forth in SEQ ID NO:122, or the complement thereof.

26. A purified polypeptide comprising an amino acid sequence that is at least 85 percent identical to the amino acid sequence set forth in SEQ ID NO:140.

27. A purified polypeptide comprising an amino acid sequence that is at least 85 percent identical to the amino acid sequence set forth in SEQ ID NO:141.

28. The purified polypeptide of claim 27, wherein said amino acid sequence is at least 90 percent identical to the amino acid sequence set forth in SEQ ID NO:141.

29. The purified polypeptide of claim 27, wherein said amino acid sequence is at least 95 percent identical to the amino acid sequence set forth in SEQ ID NO:141.

30. A purified polypeptide comprising an amino acid sequence that is at least 85 percent identical to the amino acid sequence set forth in SEQ ID NO:139.

31. The purified polypeptide of claim 30, wherein said amino acid sequence is at least 90 percent identical to the amino acid sequence set forth in SEQ ID NO:139.

32. The purified polypeptide of claim 30, wherein said amino acid sequence is at least 95 percent identical to the amino acid sequence set forth in SEQ ID NO:139.

33. A purified polypeptide comprising an amino acid sequence that is at least 85 percent identical to the amino acid sequence set forth in SEQ ID NO:142.

34. A purified polypeptide comprising an amino acid sequence that is at least 85 percent identical to the amino acid sequence set forth in SEQ ID NO:129, SEQ ID NO:130, or SEQ ID NO:131.
